(19) United States
(12) Patent Application Publication
(10) Pub. No.: US 2016/0081975 A1
(43) Pub. Date: Mar. 24, 2016

(54) SOFT GEL COMPOSITIONS AND PRE-GEL CONCENTRATES

(71) Applicant: VIRUN, INC., Walnut, CA (US)

(72) Inventor: Philip J. Bromley, Fullerton, CA (US)

(21) Appl. No.: 14/866,724

(22) Filed: Sep. 25, 2015

Related U.S. Application Data
(63) Continuation of application No. PCT/US2015/051083, filed on Sep. 18, 2015.
(60) Provisional application No. 62/052,435, filed on Sep. 18, 2014.
(60) Provisional application No. 62/052,450, filed on Sep. 18, 2014.

(51) Int. Cl. A61K 31/355; A61K 31/385; A61K 31/09; A61K 45/06; A61K 31/122; A61K 31/593; A61K 36/324; A61K 36/185; A61K 47/48; A61K 31/437

(52) U.S. Cl. CPC A61K 31/355; A61K 47/48215; A61K 31/385; A61K 31/09; A61K 31/437; A61K 31/122; A61K 31/593; A61K 36/324; A61K 36/185; A61K 45/06

(57) ABSTRACT

Provided are compositions and methods for producing non-aqueous capsule compositions that contain additives such as essential fatty acids, including omega-3 fatty acids, omega-6 fatty acids, conjugated fatty acids, and other fatty acids; phytochemicals, including phytosterols; other oils; and coenzymes, including coenzyme Q10, and other oil-based additives.

### RELATED APPLICATIONS

This application is a continuation of International PCT Application No. PCT/US2015/051083, filed Sep. 18, 2015, entitled “SOFT GEL COMPOSITIONS AND PRE-GEL CONCENTRATES,” to Philip J. Bromley, which claims priority to U.S. Provisional Application Ser. No. 62/052,435, filed Sep. 18, 2014, entitled “SOFT GEL COMPOSITIONS AND PRE-GEL CONCENTRATES,” to Philip J. Bromley, and to U.S. Provisional Application Ser. No. 62/052,450, filed Sep. 18, 2014, entitled “FORMULATIONS OF WATER-SOLUBLE DERIVATIVES OF VITAMIN E AND SOFT GEL COMPOSITIONS, CONCENTRATES AND POWDERS CONTAINING SAME,” to Philip J. Bromley.

This application is related to U.S. Application Serial No. (Attorney Docket No. 33312.05755.US02/5755), filed the same day herewith, entitled “FORMULATIONS OF WATER-SOLUBLE DERIVATIVES OF VITAMIN E AND SOFT GEL COMPOSITIONS, CONCENTRATES AND POWDERS CONTAINING SAME,” to Philip J. Bromley. U.S. Application Serial No. (Attorney Docket No. 33312.05755.US02/5755) also claims priority to U.S. Provisional Application Ser. No. 62/052,450, filed Sep. 18, 2014, entitled “FORMULATIONS OF WATER-SOLUBLE DERIVATIVES OF VITAMIN E AND SOFT GEL COMPOSITIONS, CONCENTRATES AND POWDERS CONTAINING SAME,” to Philip J. Bromley.

This application also is related to U.S. patent application Ser. No. 14/207,310, filed Mar. 12, 2014, published as US-2014-0271593-A1 on Sep. 18, 2014, entitled “FORMULATIONS OF WATER-SOLUBLE DERIVATIVES OF VITAMIN E AND COMPOSITIONS CONTAINING SAME,” which claims priority to U.S. Provisional Application Ser. No. 61/852,243, filed Mar. 15, 2013, entitled “FORMULATIONS OF PEG DERIVATIVES OF VITAMIN E AND COMPOSITIONS CONTAINING SAME,” to Philip Bromley, and International PCT Application No. PCT/US2014/25006, filed Mar. 12, 2014, published as WO 2014/151109 on Sep. 25, 2014, entitled “FORMULATIONS OF WATER-SOLUBLE DERIVATIVES OF VITAMIN E AND COMPOSITIONS CONTAINING SAME,” which also claims priority to U.S. Provisional Application Ser. No. 61/852,243, filed Mar. 15, 2013, entitled “FORMULATIONS OF PEG DERIVATIVES OF VITAMIN E AND COMPOSITIONS CONTAINING SAME” and to U.S. Provisional Application Ser. No. 61/863,732, filed Aug. 8, 2013, entitled “FORMULATIONS OF WATER-SOLUBLE DERIVATIVES OF VITAMIN E AND COMPOSITIONS CONTAINING SAME,” each to Philip Bromley.

The subject matter of each of the above-referenced applications is incorporated by reference in its entirety.

### FIELD OF THE INVENTION

Provided herein are compositions that contain high amounts of non-polar ingredients and non-aqueous solvents, for example, concentrates. Also provided are compositions for human consumption, such as soft gel capsules and other dosage forms, that contain the non-aqueous concentrates containing high amounts of non-polar ingredients and non-aqueous solvents. Methods for preparing the concentrates and soft gels are provided.

### BACKGROUND

Non-polar compounds are not readily dissolved in aqueous medium, such as water. A number of non-polar compounds are used in compositions for human consumption, for example, pharmaceuticals, nutraceuticals and/or dietary supplements. Exemplary of non-polar compounds used in such compositions are vitamins and minerals, fatty acids, and other non-polar compounds, non-polar active agents and non-polar ingredients.

Poor water solubility is a significant challenge for the delivery of non-polar compounds in products for oral human consumption. Thus, alternative delivery methods, for example soft gel capsules, that allow for sufficient bioavailability of the pharmaceuticals, nutraceuticals and/or dietary supplements contained within are desired. Encapsulation of a composition permits accurate delivery of a specified dose of non-polar ingredients in a bioavailable, easily consumed mode. Compositions containing concentrated non-polar ingredients have proven difficult to encapsulate because of their high viscosity. Thus, available compositions containing concentrated non-polar compounds in capsule form, for example, soft gel capsules, and methods for formulating such compositions, are limited.

Thus, there remains a need to develop alternate compositions containing non-polar compounds with properties, including appropriate viscosity, for encapsulation in soft gel capsules in order to administer accurate dosages, and exploit the benefits of soft gels. Also desired are methods for making the compositions, and methods for encapsulating the compositions within a soft gel, so that the soft gel capsules can be used to deliver the composition as a solid dosage form for human consumption. Accordingly, it is among the objects herein to provide compositions, including liquid and semi-solid compositions, containing concentrated non-polar compounds for containment and delivery by soft gel capsules, and methods for making compositions containing concentrated non-polar compounds for encapsulation by soft gels.

### SUMMARY

Provided are non-aqueous pre-gel concentrates that contain a water-soluble vitamin E derivative mixture or a polyethylene glycol (PEG)-derivative of vitamin E, a non-polar ingredient other than the PEG derivative of vitamin E, and a non-aqueous solvent that dissolves the non-polar ingredients and is insoluble or only partially soluble in water.

In some examples, the concentrates contain a polyalkylene glycol derivative of vitamin E, such as a polyethylene glycol (PEG)-derivative of vitamin E, typically present in an amount between 5% and 50%, 2% and 30% or 2% and 25% or 5% and 30% or 30% and 60%, inclusive, by weight of the concentrate. The PEG derivative of vitamin E typically is present in an amount between 20% and 40%, inclusive, by weight of the concentrate.

The PEG derivative of vitamin E contains a PEG moiety, for example, a PEG moiety having a molecular weight from between or between about 100 Da and 20,000 Da, 200 Da and 10,000 Da, 200 Da and 8000 Da, 200 Da and 6000 Da, 200 Da and 5000 Da, 200 Da and 3000 Da, 200 Da and 1000 Da, 200 Da and 800 Da, 200 Da and 600 Da, 200 Da and 400 Da, 400 Da and 20,000 Da, 400 Da and 10,000 Da, 400 Da and 8000 Da, 400 Da and 6000 Da, 400 Da and 5000 Da, 400 Da and 3000 Da, 400 Da and 1000 Da, 400 Da and 800 Da, 400 Da and 600 Da, 600 Da and 20,000 Da, 600 Da and 10,000 Da, 600 Da and 8000 Da, 600 Da and 6000 Da, 600 Da and 5000 Da, 600 Da and 3000 Da, 600 Da and 1000 Da, 600 Da and 800 Da, 800 Da and 20,000 Da, 800 Da and 10,000 Da, 800 Da and 8000 Da, 800 Da and 6000 Da, 800 Da and 5000 Da, 800 Da and 3000 Da, 800 Da and 1000 Da, 1000 Da and 20,000 Da, 1000 Da and 10,000 Da, 1000 Da and 8000 Da, 1000 Da and 6000 Da, 1000 Da and 5000 Da, 1000 Da and 3000 Da, 3000 Da and 20,000 Da, 3000 Da and 10,000 Da, 3000 Da and 8000 Da, 3000 Da and 6000 Da, 3000 Da and 5000 Da, 5000 Da and 20,000 Da, 5000 Da and 10,000 Da, 5000 Da and 8000 Da, 5000 Da and 6000 Da, 6000 Da and 20,000 Da, 6000 Da and 10,000 Da, 6000 Da and 8000 Da, 8000 Da and 20,000 Da, 8000 Da and 10,000 Da or 10000 Da and 20,000 Da, or has a molecular weight of 100, 200, 238, 300, 400, 500, 600, 750, 800, 1000, 1200, 1500, 2000, 2500, 3000, 3400, 3500, 4000, 6000, 8000, 10,000, 12,000 or 20,000 Da.

Among the PEG derivatives of vitamin E included in the pre-gel concentrates provided herein are tocopheryl polyethylene glycol succinate, tocopheryl polyethylene glycol sebacate, tocopheryl polyethylene glycol dodecanodioate, tocopheryl polyethylene glycol suberate, tocopheryl polyethylene glycol azelaate, tocopheryl polyethylene glycol citraconate, tocopheryl polyethylene glycol methylcitraconate, tocopheryl polyethylene glycol itaconate, tocopheryl polyethylene glycol maleate, tocopheryl polyethylene glycol glutarate, tocopheryl polyethylene glycol glutaconate, tocopheryl polyethylene glycol fumarate, tocopheryl polyethylene glycol phthalate, tocotrienol polyethylene glycol succinate, tocotrienol polyethylene glycol sebacate, tocotrienol polyethylene glycol dodecanodioate, tocotrienol polyethylene glycol suberate, tocotrienol polyethylene glycol azelaate, tocotrienol polyethylene glycol citraconate, tocotrienol polyethylene glycol methylcitraconate, tocotrienol polyethylene glycol itaconate, tocotrienol polyethylene glycol maleate, tocotrienol polyethylene glycol glutarate, tocotrienol polyethylene glycol glutaconate, tocotrienol polyethylene glycol fumarate and tocotrienol polyethylene glycol phthalate. Exemplary vitamin E derivatives are tocopheryl polyethylene glycol succinate (TPGS), tocopheryl sebacate polyethylene glycol, tocopheryl dodecanodioate polyethylene glycol, tocopheryl suberate polyethylene glycol, tocopheryl azelaate polyethylene glycol, tocopheryl citraconate polyethylene glycol, tocopheryl methylcitraconate polyethylene glycol, tocopheryl itaconate polyethylene glycol, tocopheryl maleate polyethylene glycol, tocopheryl glutarate polyethylene glycol, tocopheryl glutaconate polyethylene glycol and tocopheryl phthalate polyethylene glycol, TPGS analogs and TPGS homologs. In one example, the vitamin E derivative is D-α-tocopheryl polyethylene glycol succinate (TPGS). In another example, the vitamin E derivative is D-α-tocopheryl polyethylene glycol succinate 1000 (TPGS 1000).

In some examples, the concentrates contain a water-soluble vitamin E derivative mixture, typically present in an amount between 1% and 99%, inclusive, by weight of the concentrate. The water-soluble vitamin E derivative mixture typically is present in an amount between 20% and 40%, inclusive, by weight of the concentrate. The water-soluble vitamin E derivative mixture is a high dimer mixture that contains at least 13 wt % water-soluble vitamin E derivative dimer and up to 87% water-soluble vitamin E derivative monomer, in some examples at least 20%, 25% or 29%, by weight, water-soluble vitamin E derivative dimer. In some examples, the water-soluble vitamin E derivative mixture contains up to 75%, 70%, 69%, 62%, 55%, 50%, 45%, 40%, or 35% dimer or 29%-69%, inclusive, of dimer; and/or contains less than 70%, 65%, 63%, 62%, 61% of the vitamin E derivative monomer, such as where the amount of dimer is greater than 29% and the total amount of dimer and monomer in the water-soluble vitamin E derivative mixture is greater than 95%, 96%, 97%, 98%, or 99%.

The water-soluble vitamin E derivative mixture contains a mixture of monomers and dimers, including mixtures containing monomer at between or between about 25% and 30%, 25% and 35%, 25% and 40%, 25% and 45%, 25% and 50%, 25% and 55%, 25% and 60%, 25% and 65%, 30% and 35%, 30% and 40%, 30% and 45%, 30% and 50%, 30% and 55%, 30% and 60%, 30% and 65%, 30% and 69%, 35% and 40%, 35% and 45%, 35% and 50%, 35% and 55%, 35% and 60%, 35% and 65%, 35% and 69%, 40% and 45%, 40% and 50%, 40% and 55%, 40% and 60%, 40% and 65%, 40% and 69%, 45% and 50%, 45% and 55%, 45% and 60%, 45% and 65%, 45% and 69%, 50% and 55%, 50% and 60%, 50% and 65%, 50% and 69%, 55% and 60%, 55% and 65%, 55% and 69%, 60% and 65%, 60% and 69%, or 65% and 69%, by weight, of the water-soluble vitamin E derivative mixture or is or is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, or 68%, up to and including 69%, by weight, of the water-soluble vitamin E derivative mixture.

The water-soluble vitamin E derivative mixture contains a mixture of monomers and dimers, including mixtures containing dimer at between or between about 13% and 15%, 13% and 20%, 13% and 25%, 13% and 30%, 13% and 35%, 13% and 40%, 13% and 45%, 13% and 50%, 13% and 55%, 13% and 60%, 13% and 65%, 13% and 70%, 13% and 75%, 20% and 25%, 20% and 30%, 20% and 35%, 20% and 40%, 20% and 45%, 20% and 50%, 20% and 55%, 20% and 60%, 20% and 65%, 20% and 70%, 20% and 75%, 25% and 30%, 25% and 35%, 25% and 40%, 25% and 45%, 25% and 50%, 25% and 55%, 25% and 60%, 25% and 65%, 25% and 70%, 25% and 75%, 30% and 35%, 30% and 40%, 30% and 45%, 30% and 50%, 29% and 52%, 30% and 55%, 30% and 60%, 30% and 65%, 30% and 70%, 30% and 75%, 35% and 40%, 35% and 45%, 35% and 50%, 35% and 55%, 35% and 60%, 35% and 65%, 35% and 70%, 35% and 75%, 40% and 45%, 40% and 50%, 40% and 55%, 40% and 60%, 40% and 65%, 40% and 70%, 40% and 75%, 45% and 50%, 45% and 55%, 45% and 60%, 45% and 65%, 45% and 70%, 45% and 75%, 50% and 55%, 50% and 60%, 50% and 65%, 50% and 69%, 55% and 60%, 55% and 65%, 55% and 70%, 55% and 75%, 60% and 65%, 60% and 70%, 60% and 75%, 65% and 70%, 65% and 75%, or 70% and 75%, by weight, of the water-soluble vitamin E derivative mixture or is or is at least about 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, or 74%, up to 75%, by weight, of the water-soluble vitamin E derivative mixture.

In some examples, the monomer makes up between or between about 35% and 65%, inclusive, by weight, of the water-soluble vitamin E derivative mixture and the dimer makes up between or between about 25% and 65%, by weight, of the water-soluble vitamin E derivative mixture, or the dimer makes up between or between about 29% and 61% or 62%, by weight, of the water-soluble vitamin E derivative mixture, and the monomer and dimer together make up at least 70%, by weight, of the water-soluble vitamin E mixture in the concentrate.

The water-soluble vitamin E derivative in some examples is a polyalkylene glycol derivative of vitamin E, such as a PEG derivative of vitamin E. In some examples, the PEG moiety has a molecular weight from between or between about 100 Da and 20,000 Da, 200 Da and 10,000 Da, 200 Da and 8000 Da, 200 Da and 6000 Da, 200 Da and 5000 Da, 200 Da and 3000 Da, 200 Da and 1000 Da, 200 Da and 800 Da, 200 Da and 600 Da, 200 Da and 400 Da, 400 Da and 20,000 Da, 400 Da and 10,000 Da, 400 Da and 8000 Da, 400 Da and 6000 Da, 400 Da and 5000 Da, 400 Da and 3000 Da, 400 Da and 1000 Da, 400 Da and 800 Da, 400 Da and 600 Da, 600 Da and 20,000 Da, 600 Da and 10,000 Da, 600 Da and 8000 Da, 600 Da and 6000 Da, 600 Da and 5000 Da, 600 Da and 3000 Da, 600 Da and 1000 Da, 600 Da and 800 Da, 800 Da and 20,000 Da, 800 Da and 10,000 Da, 800 Da and 8000 Da, 800 Da and 6000 Da, 800 Da and 5000 Da, 800 Da and 3000 Da, 800 Da and 1000 Da, 1000 Da and 20,000 Da, 1000 Da and 10,000 Da, 1000 Da and 8000 Da, 1000 Da and 6000 Da, 1000 Da and 5000 Da, 1000 Da and 3000 Da, 3000 Da and 20,000 Da, 3000 Da and 10,000 Da, 3000 Da and 8000 Da, 3000 Da and 6000 Da, 3000 Da and 5000 Da, 5000 Da and 20,000 Da, 5000 Da and 10,000 Da, 5000 Da and 8000 Da, 5000 Da and 6000 Da, 6000 Da and 20,000 Da, 6000 Da and 10,000 Da, 6000 Da and 8000 Da, 8000 Da and 20,000 Da, 8000 Da and 10,000 Da or 10000 Da and 20,000 Da, or has a molecular weight of 100, 200, 238, 300, 400, 500, 600, 750, 800, 1000, 1200, 1500, 2000, 2500, 3000, 3400, 3500, 4000, 6000, 8000, 10,000, 12,000 or 20,000 Da.

Among the water-soluble vitamin E derivative mixtures included in the pre-gel concentrates provided herein are PEG derivatives of vitamin E that include tocopheryl polyethylene glycol succinate, tocopheryl polyethylene glycol sebacate, tocopheryl polyethylene glycol dodecanodioate, tocopheryl polyethylene glycol suberate, tocopheryl polyethylene glycol azelaate, tocopheryl polyethylene glycol citraconate, tocopheryl polyethylene glycol methylcitraconate, tocopheryl polyethylene glycol itaconate, tocopheryl polyethylene glycol maleate, tocopheryl polyethylene glycol glutarate, tocopheryl polyethylene glycol glutaconate, tocopheryl polyethylene glycol fumarate, tocopheryl polyethylene glycol phthalate, tocotrienol polyethylene glycol succinate, tocotrienol polyethylene glycol sebacate, tocotrienol polyethylene glycol dodecanodioate, tocotrienol polyethylene glycol suberate, tocotrienol polyethylene glycol azelaate, tocotrienol polyethylene glycol citraconate, tocotrienol polyethylene glycol methylcitraconate, tocotrienol polyethylene glycol itaconate, tocotrienol polyethylene glycol maleate, tocotrienol polyethylene glycol glutarate, tocotrienol polyethylene glycol glutaconate, tocotrienol polyethylene glycol fumarate and tocotrienol polyethylene glycol phthalate. Exemplary vitamin E derivatives are tocopheryl polyethylene glycol succinate (TPGS), tocopheryl sebacate polyethylene glycol, tocopheryl dodecanodioate polyethylene glycol, tocopheryl suberate polyethylene glycol, tocopheryl azelaate polyethylene glycol, tocopheryl citraconate polyethylene glycol, tocopheryl methylcitraconate polyethylene glycol, tocopheryl itaconate polyethylene glycol, tocopheryl maleate polyethylene glycol, tocopheryl glutarate polyethylene glycol, tocopheryl glutaconate polyethylene glycol and tocopheryl phthalate polyethylene glycol, TPGS analogs and TPGS homologs. In one example, the vitamin E derivative is D-α-tocopheryl polyethylene glycol succinate (TPGS). In another example, the vitamin E derivative is D-α-tocopheryl polyethylene glycol succinate 1000 (TPGS 1000).

In one example, the pre-gel concentrate contains the water-soluble vitamin E derivative mixture in an amount between 5% and 50%, or 2% and 30%, or 2% and 25%, or 5% and 30%, or 30% and 60%, inclusive, by weight of the concentrate; the non-polar ingredient, other than the water-soluble vitamin E derivative mixture, in an amount between 10% and 90% or 30% and 90%, inclusive, by weight of the concentrate; and the ingestible non-aqueous solvent in an amount between 2% and 20%, or 2% and 30%, or 2% and 40%, or greater than 5% up to 20%, or 30%, or 40%, or 50%, inclusive, by weight of the concentrate.

The pre-gel concentrates provided herein further contain a non-polar ingredient, other than the PEG derivative of vitamin E. In some examples, the non-polar ingredient contains one or more non-polar compounds. Exemplary non-polar compounds include polyunsaturated fatty acids (PUFAs), non-essential fatty acids, phospholipids, coenzyme Q compounds, flavonoids, carotenoids, micronutrients, Boswellia extracts, alkaloids, hops-containing compounds, antioxidants, and mixtures thereof. Exemplary PUFA-containing non-polar ingredients include, for example, fish oil, algae (algal) oil, flaxseed oil, borage oil, saw palmetto extract, safflower oil, coconut oil, soybean oil and conjugated linoleic acid (CLA)-containing compounds, where the PUFA is typically an omega-3 fatty acid, omega-6 fatty acid, omega-9 fatty acid, or conjugated fatty acid. Exemplary PUFAs include, but are not limited to, docosahexaenoic acid (DHA), eicosapentaenoic acid (EPA), alpha-linolenic acid (ALA), gamma-linolenic acid (GLA), conjugated linoleic acid (CLA), and oleic acid.

The pre-gel concentrates provided herein, in some example, contain non-polar ingredients that include, for example, a coenzyme Q10, such as ubiquinol, ubidecarenone, and ubisemiquinone; an oil-soluble vitamin, such as vitamin B12, vitamin D3, vitamin A palmitate, vitamin E, vitamin B1, vitamin B3, vitamin B5, vitamin B6, vitamin C, vitamin K2, and mixtures thereof; a carotenoid-containing compound, such as astaxanthin, lycopene, lutein, zeaxanthin, and mixtures thereof; a Boswellia extract-containing compound, such as a Boswellia serrata extract that contains acetyl-11-keto-β-boswellic acid (AKBA); a hops-containing compound, such as a supercritical extract of hops cones that includes a minimum of 30% alpha-acids (including humulone, cohumulone, adhumulone, iso-cohumulone and iso-adhumulone) and 10% beta-acids (including lupulone and colupulone); an antioxidant, such as alpha-lipoic acid, pyrroloquinoline quinone (PQQ), a turmeric/curcumin composition that is 95% curcumin, and mixtures thereof an omega-5 fatty acid derivative, such as is cetyl myristoleate (CMO); and a phospholipid, such as phosphatidylcholine.

Typically, the non-polar ingredient is present in an amount of from between 40% to 90%, by weight of the composition, or is 40%-55%, 40%-70%, 40%-85%, 50%-65%, 50%-70%, 50%-75%, 50%-85%, 60%-70%, 60%-80%, 60%-90%, 70%-85%, 70%-90%, or 80%-90%, inclusive, by weight of the concentrate. For example, the non-polar ingredient, other than the PEG derivative of vitamin E, typically is present in an amount between 10% and 90% or 30% and 90%, inclusive, by weight of the concentrate.

The concentrates provided herein contain a non-aqueous solvent. Typically, the non-aqueous solvent is an alcohol, an alcohol derivative, a hydrocarbon, or mixtures thereof, such as an aromatic alcohol, an ester derivative of an alcohol, a cyclic hydrocarbon, or mixtures thereof, for example, benzyl alcohol, benzyl benzoate, d-limonene, or mixtures thereof. The non-aqueous solvent is typically present in an amount between 2% and 20%, or 2% and 30%, or 2% and 40%, or greater than 5% up to 20%, or 30%, or 40%, or 50%, inclusive, by weight of the concentrate, such as between 2% and 20%, inclusive, by weight of the concentrate. In some examples, the non-aqueous solvent is present in an amount of at least 10%, up to and including 30%; or at least 5%, up to and including 20%; at least 7%, up to and including 20%; or at least 10%, up to and including 20%; or at least 7%, up to and including 15%.

In some examples, the non-aqueous solvent is selected from among alcohols, alcohol derivatives, alkanes, including hydrocarbons and cyclic hydrocarbons, including d-limonene; aromatic compounds, including aromatic alcohols, aromatic ethers and aromatic esters, including benzyl alcohol and benzyl benzoate; haloalkanes, ethers, esters and ketones; organic solvents of natural origin, including natural hydrocarbons, including terpenes, including d-limonene, alpha-pinene, beta-pinene, myrcene, linalol, citronellol, geraniol, menthol, citral, citronellal, or oxidized organic derivatives, including ethers, aldehydes, alcohols and esters; lactams, including N-methyl pyrrolidone (NMP); alkylene glycols, including propylene glycol and polyethylene glycol, including PEG300 and PEG400; dimethyl sulfoxide; dimethyl acetamide; 2-pyrrolidone; C2-C6 alkanols; 2-ethoxyethanol; alkyl esters including 2-ethoxyethyl acetate, methyl acetate, ethyl acetate, ethylene glycol diethyl ether, and ethylene glycol dimethyl ether; (S)-(−)-ethyl lactate; acetone; glycerol; alkyl ketones including methylethyl ketone or dimethyl sulfone; tetrahydrofuran; cyclic alkyl amides including caprolactam; decylmethylsulfoxide; oleic acid; aromatic amines including N,N-diethyl-m-toluamide; 1 dodecylazacycloheptan-2-one; natural products, including olive oils and saturated and non-saturated fatty acids; acyl glycerols; and mixtures thereof.

In one example, the pre-gel concentrate contains a non-aqueous solvent, for example, benzyl alcohol, d-limonene, or a mixture thereof, in an amount between 7% and 16%, inclusive, by weight of the concentrate; a polyethylene glycol (PEG)-derivative of vitamin E, for example, D-α-tocopheryl polyethylene glycol succinate (TPGS), or a water-soluble vitamin E derivative mixture that is a high dimer PEG derivative of vitamin E mixture that is a high dimer TPGS mixture, in an amount between 25% and 35%, inclusive, by weight of the concentrate; and a non-polar ingredient in an amount between 53% and 66%, inclusive, by weight of the concentrate. The non-polar ingredient is, for example, one or more of a coenzyme Q10, such as ubiquinol, ubidecarenone, and ubisemiquinone; an oil-soluble vitamin, such as vitamin B12, vitamin D3, vitamin A palmitate, vitamin E, vitamin B1, vitamin B3, vitamin B5, vitamin B6, vitamin C, vitamin K2, and mixtures thereof; a carotenoid-containing compound, such as astaxanthin, lycopene, lutein, zeaxanthin, and mixtures thereof; a Boswellia extract-containing compound, such as a Boswellia serrata extract that contains acetyl-11-keto-β-boswellic acid (AKBA); a hops-containing compound, such as a supercritical extract of hops cones that includes a minimum of 30% alpha-acids (including humulone, cohumulone, adhumulone, iso-cohumulone and iso-adhumulone) and 10% beta-acids (including lupulone and colupulone); an antioxidant, such as alpha-lipoic acid, pyrroloquinoline quinone (PQQ), a turmeric/curcumin composition that is 95% curcumin, and mixtures thereof; an omega-5 fatty acid derivative, such as cetyl myristoleate (CMO); and a phospholipid that is a phosphatidylcholine.

In some examples, the pre-gel concentrate includes a non-aqueous solvent that is a mixture of benzyl alcohol and d-limonene, in an amount between 7% and 16%, inclusive, by weight of the concentrate; a polyethylene glycol (PEG)-derivative of vitamin E that is D-α-tocopheryl polyethylene glycol succinate (TPGS), or a water-soluble vitamin E derivative mixture that is a high dimer PEG derivative of vitamin E mixture that is a high dimer TPGS mixture, in an amount between 25% and 30%, inclusive, by weight of the concentrate; and a non-polar ingredient in an amount between 60% and 66%, inclusive, by weight of the concentrate. In typical examples, the non-polar ingredient includes a mixture of vitamin D3, a Boswellia serrata extract that contains acetyl-11-keto-β-boswellic acid (AKBA), a hops-containing compound that contains a supercritical extract of hops cones that includes a minimum of 30% alpha-acids (including humulone, cohumulone, adhumulone, iso-cohumulone and iso-adhumulone) and 10% beta-acids (including lupulone and colupulone), a turmeric/curcumin composition that is 95% curcumin, cetyl myristoleate (CMO), and phosphatidylcholine.

In another example, the pre-gel concentrate includes a non-aqueous solvent that is benzyl alcohol, in an amount between 12% and 17%, inclusive, by weight of the concentrate; a polyethylene glycol (PEG)-derivative of vitamin E that is D-α-tocopheryl polyethylene glycol succinate (TPGS), or a water-soluble vitamin E derivative mixture that is a high dimer PEG derivative of vitamin E mixture that is a high dimer TPGS mixture, in an amount between 25% and 30%, inclusive, by weight of the concentrate; and a non-polar ingredient in an amount between 53% and 58%, inclusive, by weight of the concentrate. Typically, the non-polar ingredient includes a mixture of vitamin E oil, vitamin D3, vitamin K2, borage oil, alpha-lipoic acid, ubiquinol, pyrroloquinoline quinone (PQQ), and astaxanthin.

In yet another example, the pre-gel concentrate includes a non-aqueous solvent that is benzyl alcohol, in an amount between 10% and 12%, inclusive, by weight of the concentrate; a polyethylene glycol (PEG)-derivative of vitamin E that is D-α-tocopheryl polyethylene glycol succinate (TPGS), or a water-soluble vitamin E derivative mixture that is a high dimer PEG derivative of vitamin E mixture that is a high dimer TPGS mixture, in an amount between 28% and 35%, inclusive, by weight of the concentrate; and a non-polar ingredient in an amount between 52% and 62%, inclusive, by weight of the concentrate. Typically, the non-polar ingredient is oleic acid. In one example, the non-polar ingredient further includes vitamin E oil and astaxanthin. In another example, the non-polar ingredient further includes cetyl myristoleate, vitamin D3, and a Boswellia extract-containing compound that is a Boswellia serrata extract that contains acetyl-11-keto-β-boswellic acid (AKBA).

In another example, the pre-gel concentrate includes a non-aqueous solvent in an amount between 2% and 20%, or 2% and 30%, or 2% and 40%, or greater than 5% up to 20%, or 30%, or 40%, or 50%, inclusive, by weight of the concentrate; a polyethylene glycol (PEG) derivative of vitamin E or a water-soluble vitamin E derivative mixture that is a high dimer vitamin E derivative mixture in an amount between 5% and 50%, 2% and 30% or 2% and 25% or 5% and 30% or 30% and 60%, inclusive, by weight of the concentrate; and a non-polar ingredient, other than the PEG-derivative of vitamin E or water-soluble vitamin E derivative mixture, in an amount between 10% and 90% or 30% and 90%, inclusive, by weight of the concentrate

The pre-gel concentrates provided herein can additionally contain one or more ingredients, such as a co-surfactant, e.g., a phospholipid, a sugar-derived surfactant, or a PEG-derived surfactant.

Typically, the pre-gel concentrates are non-aqueous.

Provided herein are capsule compositions that contain the non-aqueous pre-gel concentrates provided herein, encapsulated in a shell or coating. For example, provided herein is a capsule composition that contains a composition encapsulated in a shell or coating. They encapsulated composition includes a non-aqueous solvent in an amount greater than 2% and up to 50% inclusive, by weight of the composition; a polyethylene glycol (PEG)-derivative of vitamin E or water-soluble vitamin E derivative mixture that is a high dimer PEG derivative of vitamin E; and a non-polar ingredient other than the PEG derivative of vitamin E or water-soluble vitamin E derivative mixture. In some examples, the capsule is a soft gel.

In some examples, the provided capsule compositions contain a composition that includes a non-aqueous solvent in an amount between 2% and 20%, or 2% and 30%, or 2% and 40%, or greater than 5% up to 20%, or 30%, or 40%, or 50%, inclusive, by weight of the concentrate; a polyethylene glycol (PEG)-derivative of vitamin E or a high dimer PEG derivative of vitamin E mixture in an amount between 5% and 50%, 2% and 30% or 2% and 25% or 5% and 30% or 30% and 60%, inclusive, by weight of the concentrate; and a non-polar ingredient other than the PEG derivative of vitamin E or high dimer PEG derivative of vitamin E mixture in an amount between 10% and 90% or 30% and 90%, inclusive, by weight of the concentrate. The composition is encapsulated in a shell or coating. In some examples, the shell or coating is a soft gel capsule.

In the soft gel compositions provided herein, the non-aqueous solvent is insoluble or only partially soluble in water and dissolves the non-polar compounds or ingredients. The non-aqueous solvent is typically an alcohol, an alcohol derivative, a hydrocarbon, or mixtures thereof, such as an aromatic alcohol, an ester derivative of an alcohol, a cyclic hydrocarbon, or mixtures thereof, for example, benzyl alcohol, benzyl benzoate, d-limonene, or mixtures thereof. Typically, the non-aqueous solvent does not dissolve the shell or coating.

The capsule compositions contain a PEG derivative of vitamin E or a high dimer PEG derivative of vitamin E mixture that contains a PEG moiety having, for example, a molecular weight from between or between about 100 Da and 20,000 Da, 200 Da and 10,000 Da, 200 Da and 8000 Da, 200 Da and 6000 Da, 200 Da and 5000 Da, 200 Da and 3000 Da, 200 Da and 1000 Da, 200 Da and 800 Da, 200 Da and 600 Da, 200 Da and 400 Da, 400 Da and 20,000 Da, 400 Da and 10,000 Da, 400 Da and 8000 Da, 400 Da and 6000 Da, 400 Da and 5000 Da, 400 Da and 3000 Da, 400 Da and 1000 Da, 400 Da and 800 Da, 400 Da and 600 Da, 600 Da and 20,000 Da, 600 Da and 10,000 Da, 600 Da and 8000 Da, 600 Da and 6000 Da, 600 Da and 5000 Da, 600 Da and 3000 Da, 600 Da and 1000 Da, 600 Da and 800 Da, 800 Da and 20,000 Da, 800 Da and 10,000 Da, 800 Da and 8000 Da, 800 Da and 6000 Da, 800 Da and 5000 Da, 800 Da and 3000 Da, 800 Da and 1000 Da, 1000 Da and 20,000 Da, 1000 Da and 10,000 Da, 1000 Da and 8000 Da, 1000 Da and 6000 Da, 1000 Da and 5000 Da, 1000 Da and 3000 Da, 3000 Da and 20,000 Da, 3000 Da and 10,000 Da, 3000 Da and 8000 Da, 3000 Da and 6000 Da, 3000 Da and 5000 Da, 5000 Da and 20,000 Da, 5000 Da and 10,000 Da, 5000 Da and 8000 Da, 5000 Da and 6000 Da, 6000 Da and 20,000 Da, 6000 Da and 10,000 Da, 6000 Da and 8000 Da, 8000 Da and 20,000 Da, 8000 Da and 10,000 Da or 10000 Da and 20,000 Da, or has a molecular weight of 100, 200, 238, 300, 400, 500, 600, 750, 800, 1000, 1200, 1500, 2000, 2500, 3000, 3400, 3500, 4000, 6000, 8000, 10,000, 12,000 or 20,000 Da.

Among the PEG derivatives of vitamin E or high dimer PEG derivative of vitamin E mixture included in the soft gel compositions provided herein are tocopheryl polyethylene glycol succinate, tocopheryl polyethylene glycol sebacate, tocopheryl polyethylene glycol dodecanodioate, tocopheryl polyethylene glycol suberate, tocopheryl polyethylene glycol azelaate, tocopheryl polyethylene glycol citraconate, tocopheryl polyethylene glycol methylcitraconate, tocopheryl polyethylene glycol itaconate, tocopheryl polyethylene glycol maleate, tocopheryl polyethylene glycol glutarate, tocopheryl polyethylene glycol glutaconate, tocopheryl polyethylene glycol fumarate, tocopheryl polyethylene glycol phthalate, tocotrienol polyethylene glycol succinate, tocotrienol polyethylene glycol sebacate, tocotrienol polyethylene glycol dodecanodioate, tocotrienol polyethylene glycol suberate, tocotrienol polyethylene glycol azelaate, tocotrienol polyethylene glycol citraconate, tocotrienol polyethylene glycol methylcitraconate, tocotrienol polyethylene glycol itaconate, tocotrienol polyethylene glycol maleate, tocotrienol polyethylene glycol glutarate, tocotrienol polyethylene glycol glutaconate, tocotrienol polyethylene glycol fumarate and tocotrienol polyethylene glycol phthalate. Exemplary vitamin E derivatives are tocopheryl polyethylene glycol succinate (TPGS), tocopheryl sebacate polyethylene glycol, tocopheryl dodecanodioate polyethylene glycol, tocopheryl suberate polyethylene glycol, tocopheryl azelaate polyethylene glycol, tocopheryl citraconate polyethylene glycol, tocopheryl methylcitraconate polyethylene glycol, tocopheryl itaconate polyethylene glycol, tocopheryl maleate polyethylene glycol, tocopheryl glutarate polyethylene glycol, tocopheryl glutaconate polyethylene glycol and tocopheryl phthalate polyethylene glycol, TPGS analogs and TPGS homologs. In one example, the vitamin E derivative is D-α-tocopheryl polyethylene glycol succinate (TPGS). In another example, the vitamin E derivative is D-α-tocopheryl polyethylene glycol succinate 1000 (TPGS 1000).

In some examples, the polyalkylene glycol derivative of vitamin E is a high dimer containing water soluble vitamin E derivative composition, such as a high dimer mixture, for example, a high dimer PEG derivative of vitamin E composition, e.g., a high dimer D-α-tocopheryl polyethylene glycol succinate (TPGS) composition.

Typically, the PEG derivative of vitamin E is present in an amount between 20% and 40%, inclusive, by weight of the composition.

The capsule compositions contain a non-polar ingredient. In some examples, the non-polar ingredient contains one or more non-polar compounds. Exemplary non-polar compounds include polyunsaturated fatty acids (PUFAs), non-essential fatty acids, phospholipids, coenzyme Q compounds, flavonoids, carotenoids, micronutrients, Boswellia extracts, alkaloids, hops-containing compounds, antioxidants, and mixtures thereof. Exemplary PUFA-containing non-polar ingredients include, for example, fish oil, algae (algal) oil, flaxseed oil, borage oil, saw palmetto extract, safflower oil, coconut oil, soybean oil and conjugated linoleic acid (CLA)-containing compounds, where the PUFA is typically an omega-3 fatty acid, omega-6 fatty acid, omega-9 fatty acid, or conjugated fatty acid. Exemplary PUFAs include, but are not limited to, docosahexaenoic acid (DHA), eicosapentaenoic acid (EPA), alpha-linolenic acid (ALA), gamma-linolenic acid (GLA), conjugated linoleic acid (CLA), and oleic acid.

The capsule compositions provided herein, in some example, contain non-polar ingredients that include, for example, a coenzyme Q10, such as ubiquinol, ubidecarenone, and ubisemiquinone; an oil-soluble vitamin, such as vitamin B12, vitamin D3, vitamin A palmitate, vitamin E, vitamin B1, vitamin B3, vitamin B5, vitamin B6, vitamin C, vitamin K2, and mixtures thereof; a carotenoid-containing compound, such as astaxanthin, lycopene, lutein, zeaxanthin, and mixtures thereof; a Boswellia extract-containing compound, such as a Boswellia serrata extract that contains acetyl-11-keto-β-boswellic acid (AKBA); a hops-containing compound, such as a supercritical extract of hops cones that includes a minimum of 30% alpha-acids (including humulone, cohumulone, adhumulone, iso-cohumulone and iso-adhumulone) and 10% beta-acids (including lupulone and colupulone); an antioxidant, such as alpha-lipoic acid, pyrroloquinoline quinone (PQQ), a turmeric/curcumin composition that is 95% curcumin, and mixtures thereof; an omega-5 fatty acid derivative, such as is cetyl myristoleate (CMO); and a phospholipid, such as phosphatidylcholine.

Typically, the non-polar ingredient is present in an amount of from between 40% to 90%, by weight of the concentrate, or is 40%-55%, 40%-70%, 40%-85%, 50%-65%, 50%-70%, 50%-75%, 50%-85%, 60%-70%, 60%-80%, 60%-90%, 70%-85%, 70%-90%, or 80%-90%, inclusive, by weight of the concentrate.

In some example, the capsule composition includes a composition containing a non-aqueous solvent such as benzyl alcohol, d-limonene, or a mixture thereof, in an amount between 7% and 16%, inclusive, by weight of the composition; a polyethylene glycol (PEG)-derivative of vitamin E that is D-α-tocopheryl polyethylene glycol succinate (TPGS) or a water-soluble vitamin E derivative mixture that is a high dimer PEG derivative of vitamin E mixture that is a high dimer TPGS mixture, in an amount between 25% and 35%, inclusive, by weight of the composition; and a non-polar ingredient in an amount between 53% and 66%, inclusive, by weight of the composition. Exemplary non-polar ingredients included in the composition are a coenzyme Q10, such as ubiquinol, ubidecarenone, and ubisemiquinone; an oil-soluble vitamin such as vitamin B12, vitamin D3, vitamin A palmitate, vitamin E, vitamin B1, vitamin B3, vitamin B5, vitamin B6, vitamin C, vitamin K2, and mixtures thereof; a carotenoid-containing compound such as astaxanthin, lycopene, lutein, zeaxanthin, and mixtures thereof; a Boswellia extract-containing compound such as a Boswellia serrata extract that contains acetyl-11-keto-β-boswellic acid (AKBA); a hops-containing compound that contains a supercritical extract of hops cones that includes a minimum of 30% alpha-acids (including humulone, cohumulone, adhumulone, iso-cohumulone and iso-adhumulone) and 10% beta-acids (including lupulone and colupulone); an antioxidant such as alpha-lipoic acid, pyrroloquinoline quinone (PQQ), a turmeric/curcumin composition that is 95% curcumin, and mixtures thereof; an omega-5 fatty acid derivative such as cetyl myristoleate (CMO); and a phospholipid such as phosphatidylcholine. The composition is encapsulated by a shell or coating, for example, a gelatin shell or coating.

In one example, the capsule composition includes a composition that contains a non-aqueous solvent that is a mixture of benzyl alcohol and d-limonene, in an amount between 7% and 16%, inclusive, by weight of the concentrate; a polyethylene glycol (PEG)-derivative of vitamin E that is D-α-tocopheryl polyethylene glycol succinate (TPGS) or a water-soluble vitamin E derivative mixture that is a high dimer PEG derivative of vitamin E mixture that is a high dimer TPGS mixture, in an amount between 25% and 30%, inclusive, by weight of the concentrate; and a non-polar ingredient in an amount between 60% and 66%, inclusive, by weight of the concentrate. The non-polar ingredient includes a mixture of vitamin D3, a Boswellia serrata extract that contains acetyl-11-keto-β-boswellic acid (AKBA), a hops-containing compound that contains a supercritical extract of hops cones that includes a minimum of 30% alpha-acids (including humulone, cohumulone, adhumulone, iso-cohumulone and iso-adhumulone) and 10% beta-acids (including lupulone and colupulone), a turmeric/curcumin composition that is 95% curcumin, cetyl myristoleate (CMO), and phosphatidylcholine. The composition is encapsulated in a shell or coating, such as a gelatin shell or coating.

In another example, the capsule composition includes a composition containing a non-aqueous solvent that is benzyl alcohol, in an amount between 12% and 17%, inclusive, by weight of the concentrate; a polyethylene glycol (PEG)-derivative of vitamin E that is D-α-tocopheryl polyethylene glycol succinate (TPGS) or a water-soluble vitamin E derivative mixture that is a high dimer PEG derivative of vitamin E mixture that is a high dimer TPGS mixture, in an amount between 25% and 30%, inclusive, by weight of the concentrate; and a non-polar ingredient in an amount between 53% and 58%, inclusive, by weight of the concentrate. The non-polar ingredient includes a mixture of vitamin E oil, vitamin D3, vitamin K2, borage oil, alpha-lipoic acid, ubiquinol, pyrroloquinoline quinone (PQQ), and astaxanthin. The composition is encapsulated in a shell or coating, such as a gelatin shell or coating.

In another example, the capsule composition includes a composition containing a non-aqueous solvent that is benzyl alcohol, in an amount between 10% and 12%, inclusive, by weight of the concentrate; a polyethylene glycol (PEG)-derivative of vitamin E that is D-α-tocopheryl polyethylene glycol succinate (TPGS) or a water-soluble vitamin E derivative mixture that is a high dimer PEG derivative of vitamin E mixture that is a high dimer TPGS mixture, in an amount between 28% and 35%, inclusive, by weight of the concentrate; and a non-polar ingredient in an amount between 52% and 62%, inclusive, by weight of the concentrate. The non-polar ingredient is oleic acid. The composition is encapsulated in a shell or coating, such as a gelatin shell or coating. In one example, the non-polar ingredient further includes vitamin E oil and astaxanthin. In another example, the non-polar ingredient further includes cetyl myristoleate, vitamin D3, and a Boswellia extract-containing compound that is a Boswellia serrata extract that contains acetyl-11-keto-β-boswellic acid (AKBA).

The capsule compositions, in some examples, further include a co-surfactant such as a phospholipid, a sugar-derived surfactant, and a PEG-derived surfactant.

The shell or coating is typically a gelatin or gelatin substitute shell or coating, for example, natural gelatin, synthetic gelatin, pectin, casein, collagen, protein, modified starch, polyvinyl pyrrolidone, acrylic, natural or synthetic polymers, a cellulose derivative that is hydroxypropyl methylcellulose (HPMC), seaweed extract, and combinations thereof.

The shell or coating in some examples includes one or more of water, opacifiers, plasticizers, preservatives, coloring agents, flavoring agents, sweeteners, sugars, gastroresistant substances, fillers, binders, lubricants, or disintegrants. In some examples, the shell or coating is coated with an immediate release coating, protective coating, enteric or delayed release coating, sustained release coating, barrier coating, or combination thereof. The shell or coating can be a one-piece shell or coating or a two-piece shell or coating and can be any size, for example, a one-piece shell or coating that is a size between or is size 1-120 or is size 1, 2, 3, 4, 5, 6, 7, 7.5, 8, 8.5, 9, 9.5, 10, 11, 12, 14, 15, 16, 17.5, 20, 22, 24, 28, 30, 40, 45, 55, 60, 65, 80, 90, or 120, or a two-piece shell or coating that is a size selected from among 5, 4, 3, 2, 1, 0, 00, and 000. The volume of the shell or coating can hold typically is 0.1-1 mL, 0.2-0.9 mL, 0.3-0.8 mL, 0.3-0.7 mL, 0.3-0.5 mL, 0.3 mL, 0.5 mL, 0.7 mL, up to 1 mL. In other examples, the shell or coating holds between about or is 10 mg to 1000 mg, 30 mg to 800 mg, 50 mg to 600 mg, 70 mg to 500 mg, 100 mg to 300 mg, or at least 30, 40, 50, 60, 70, 80, 90. 100, 150, 200, 300, 400, 500, 600, 800 or 1000 mg. Capsules include capsules of any shape, for example, round, oval, oblong, tube or a special shape.

The capsule compositions provided here are typically non-aqueous and formulated for oral administration, for example, for human consumption. In a typical example, after consumption, the shell or coating dissolves and releases the contents.

Provided herein are methods of making the pre-gel concentrates and capsule compositions provided herein. In an exemplary method, the ingredients are first mixed and heated ingredients in a vessel, wherein the ingredients include a non-aqueous solvent in an amount between 2% and 30% or 2% and 40% or greater than 5% up to 40% or 50%, inclusive, by weight of the concentrate; a polyethylene glycol (PEG)-derivative of vitamin E or a water-soluble vitamin E derivative mixture that is a high dimer PEG derivative of vitamin E mixture in an amount between 5% and 50%, 2% and 30% or 2% and 25% or 5% and 30% or 30% and 60%, inclusive, by weight of the concentrate; and a non-polar ingredient other than the PEG derivative of vitamin E in an amount between 10% and 90% or 30% and 90%, inclusive, by weight of the concentrate. After mixing and heating, the ingredients are homogenized, then cooled, thus forming the liquid concentrate.

In one method, for example, the non-aqueous solvent is an alcohol, an alcohol derivative, a hydrocarbon, or mixtures thereof; the PEG derivative of vitamin E is tocopheryl polyethylene glycol succinate (TPGS), tocopheryl sebacate polyethylene glycol, tocopheryl dodecanodioate polyethylene glycol, tocopheryl suberate polyethylene glycol, tocopheryl azelaate polyethylene glycol, tocopheryl citraconate polyethylene glycol, tocopheryl methylcitraconate polyethylene glycol, tocopheryl itaconate polyethylene glycol, tocopheryl maleate polyethylene glycol, tocopheryl glutarate polyethylene glycol, tocopheryl glutaconate polyethylene glycol and tocopheryl phthalate polyethylene glycol, TPGS analogs or TPGS homologs; and the non-polar ingredient includes one or more of a coenzyme Q10, such as ubiquinol, ubidecarenone, and ubisemiquinone; an oil-soluble vitamin including vitamin B12, vitamin D3, vitamin A palmitate, vitamin E, vitamin B1, vitamin B3, vitamin B5, vitamin B6, vitamin C, vitamin K2, and mixtures thereof; a carotenoid-containing compound including astaxanthin, lycopene, lutein, zeaxanthin, and mixtures thereof; a Boswellia extract-containing compound that is a Boswellia serrata extract that contains acetyl-11-keto-β-boswellic acid (AKBA); a hops-containing compound that contains a supercritical extract of hops cones that includes a minimum of 30% alpha-acids (including humulone, cohumulone, adhumulone, iso-cohumulone and iso-adhumulone) and 10% beta-acids (including lupulone and colupulone); an antioxidant including alpha-lipoic acid, pyrroloquinoline quinone (PQQ), a turmeric/curcumin composition that is 95% curcumin, and mixtures thereof; an omega-5 fatty acid derivative that is cetyl myristoleate (CMO); and a phospholipid that is a phosphatidylcholine.

In an exemplary method, the pre-gel concentrate is introduced into a soft gel shell or capsule, such as a gelatin gel or capsule. In some examples, the method include introducing the concentrate into the soft gel shell or capsule by the plate process, the rotary die process, the reciprocating die process, the bubble process, or the continuous process.

### DETAILED DESCRIPTION

### Outline

A. Definitions
B. Compositions containing non-polar ingredients
   1. Non-aqueous pre-gel compositions containing non-polar ingredients
      a. Non-polar ingredients
         1) Fatty acids
            a) Polyunsaturated fatty acid (PUFA)-containing ingredients
            i. Omega-3 fatty acid compounds
            (1) DHA/EPA
            (a) Fish oils
            (b) Algae oil
            (2) Flax seed oil—omega 3 (ALA)
            ii. Omega-6 fatty acid compounds
            iii. Saw palmetto extract
            iv. Conjugated linoleic acid (CLA)
            b) Non-essential fatty acids
            c) Phospholipids
         2) Coenzyme Q compounds
         3) Phytochemical-containing compounds
            a) Phytosterols
            b) Flavonoids
         4) Carotenoid-containing compounds
            a) Carotenes
            b) Xanthophylls
         5) Micronutrient-containing compounds
            a) Vitamins
         6) Boswellia extracts
         7) Alkaloids
         8) Cannabinoids
         9) Hops-containing compounds
         10) Antioxidants
         11) Additional non-polar compounds
      b. Surfactants
         1) Polyalkylene glycol derivatives of vitamin E
            a) Tocopherols and tocotrienols
            b) Linkers
            c) PEG moieties
         2) Tocopheryl polyalkylene glycol derivatives
            a) Synthesis
            b) Water-soluble vitamin E derivative mixtures (compositions)
         3) Methods for making water-soluble vitamin E derivatives
            a) Reaction mixture
            i. Vitamin E succinate
            ii. Polyethylene glycol
            iii. Catalyst
            iv. Solvent
            v. Exemplary reaction mixtures
            b) Exemplary methods
            i. Preparation of a crude water-soluble vitamin E derivative mixture
            ii. Processing the reaction mixture to obtain a crude water-soluble vitamin E derivative mixture
            iii. Purification of the crude water-soluble vitamin E derivative mixture to obtain a purified high dimer-containing water-soluble vitamin E derivative mixture
         4) Surfactant properties
      c. Non-aqueous solvents
      d. Other ingredients
         1) Co-surfactants
            i. Phospholipids
            ii. Sugar-derived surfactants
            iii. PEG-derived surfactants
         2) Flavors
   2. Formulating the non-aqueous pre-gel compositions containing non-polar ingredients
   3. Soft gel compositions containing non-polar ingredients
      a. Capsules
      b. Formulating the soft gel compositions
      c. Ingredients and concentration ranges
      d. Exemplary dosages and administration of the soft gel compositions
C. Exemplary methods for preparing non-aqueous pre-gel concentrates containing non-polar ingredients
   1. Equipment employed in the methods
      a. Scales
      b. Purifiers
      c. Vessels
      d. Mixers
      e. Heating/cooling apparatuses
      f. Transfer devices
      g. Evaluation equipment
   2. General methods for preparing non-aqueous pre-gel concentrates
      a. Ingredients
      b. Production of the non-aqueous pre-gel concentrates
      c. Transfer and/or packaging
D. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, a soft gel shell is an oral dosage form that administration of pharmaceuticals and nutraceuticals, which are introduced or formulated in the shell, similar to capsules. Soft gel shells typically contain a combination of gelatin or gelatin alternative, such as vegan alternative, water, opacifier, and a plasticizer, such as glycerin and/or sorbitol, to lend flexibility.

As used herein, “soft gel composition” refers to a soft gel shell that contains the non-aqueous pre-gel compositions provided herein. A soft gel composition can be a capsule designed for oral administration. Typically, the capsule contains an amount of the pre-gel composition to provide a single dosage or fractional amount of the non-polar ingredients.

As used herein, a composition is a mixture of two or more ingredients.

As used herein, a combination is an association of two or more elements, such as two agents that are administered together. A kit is a packaged combination.

As used herein, “non-aqueous solvent” refers to any liquid other than water in which non-polar compounds and ingredients can be dissolved. Typically, a non-aqueous solvent is not soluble in water or is partially soluble in water or has no or only partial solubility in water. For purposes herein, the non-aqueous solvent is acceptable for human consumption. Benzyl alcohol is exemplary of such solvents.

As used herein, “alcohol derivative” includes an alcohol compound that has been modified by one or several chemical reactions. For example, the alcohol can be esterified to form an ester or oxidized to a carbonyl compound such as an aldehyde or carboxylic acid. Alcohol derivatives include alcohols protected with a suitable protecting group known to those of skill in the art. Other derivatives can include ethers, alcohol alkoxylates and mixed alkoxylates, and alcohol acid esters. One of skill in the art will appreciate that other alcohol derivatives are useful in the present invention. For purposes herein, the alcohol derivatives are non-aqueous solvents acceptable for human consumption. Benzyl benzoate is exemplary of such an alcohol derivative.

As used herein, “vitamin E” refers to any naturally occurring or synthetic form of vitamin E, for example, tocopherols and tocotrienols, and can refer to a single form of the compound or a mixture of forms.

As used herein, “water-soluble vitamin E derivative composition,” “water-soluble vitamin E derivative,” “water-soluble vitamin E derivative surfactant,” “water-soluble vitamin E surfactant,” and “water-soluble derivative of vitamin E mixture,” which are be used interchangeably, refer to compositions that contain mixtures of water-soluble forms of vitamin E (vitamin E derivatized with moieties, such as polyalkylene glycol that increase the water solubility of the water-insoluble vitamin E). A “polyalkylene glycol derivative of vitamin E” is thus a water-soluble vitamin E derivative composition that contains a mixture of water-soluble forms of vitamin E and is derivatized with a polyalkylene glycol moiety.

The mixtures can contain dimers and monomers of the vitamin E derivatives. The water-soluble vitamin E derivative mixtures (compositions) include vitamin E (natural or synthetic forms of vitamin E), such as tocopherol derivatives and tocotrienol derivatives. Generally vitamin E derivative mixtures contain predominantly or primarily monomer forms. Derivatives of vitamin E, such as polyethylene glycol (PEG)-derivatives previously produced, are manufactured to contain as much monomer form as possible, and to contain minimal amounts of any dimer form (see, e.g., Christiansen et al. (2011) J. Pharm. Sci. 100(5):1773-1782). All are intended to be included in the compositions herein.

In contrast, the high dimer-containing vitamin E derivative mixtures, such as PEG derivative of vitamin E compositions (also referred to herein as high dimer PEG derivatives of vitamin E mixtures) can be employed herein. These mixtures are manufactured to contain dimer forms, and they contain at least 13%, particularly at least or at least about 20%, 25%, 29%, or more, dimer form of the water-soluble vitamin E derivative. In particular, the water-soluble vitamin E derivative mixtures (compositions) are manufactured to contain between or between about 13 wt % and about or up to 95%, 90%, 85%, 80%, or 75 wt %, particularly at least 29% to 75% or 80%, inclusive, of the water-soluble vitamin E dimer. In general, the high dimer-containing derivatives, such as PEG derivatives of vitamin E mixtures, such as a high dimer TPGS composition, contain 30% to 60%, particularly 35% to 52%, dimer, and the remainder is the monomer form and other trace components, such as unreacted reagents, such as vitamin E and the hydrophilic derivatizing moiety.

In general, for the high dimer mixtures, the mixtures contain at least 13% of the dimer form and up to 87% monomer form, particularly at least 25% of the dimer form and up to 70% of the monomer form, such as between or between about 25 wt % and 69%, inclusive, of the monomer. Hence, the water-soluble vitamin E derivative mixtures (compositions) (high dimer containing compositions) contain a substantial amount (i.e., 13% or more, particularly 25%, 29%, 35%, 48%, 52%, or more) of the dimer form compared to commercially available forms that are manufactured to provide the monomer form.

As manufactured, the high dimer containing mixtures can include other forms and unreacted components, hence the total amount of dimer and monomer do not necessarily total 100%, by weight, of the composition. It is shown herein that inclusion of at least 13%, 20%, 25%, 29%, or more of the dimer form, and some monomer form, about less than 87%, 69%, 65%, 60%, 55%, or 50% of the monomer with at least 13% dimer, confers advantageous properties on these water-soluble vitamin E derivative mixtures (compositions) not possessed by such compositions that contain lower amounts of the dimer form.

Examples of water-soluble vitamin E derivatives are those formed by covalently attaching a vitamin E moiety, e.g., a tocopherol or tocotrienol, to a hydrophilic moiety, for example, an alkylene glycol, such as a polyethylene glycol (PEG) moiety, via a linker. The compositions include those that are commercially available, manufactured to maximize the concentration of monomer (such as those sold by Eastman), and those that are manufactured so that the resulting water-soluble vitamin E derivative mixtures (compositions) include a mixture of monomers and dimers of the water-soluble vitamin E derivatives (see, e.g.,U.S. patent application Ser. No. 13/815,193, and International Application No. PCT/US2013/25445, which describe such mixtures), and contain a substantial amount (compared to prior art preparations), i.e., 13% to 95%, inclusive, such as at least 13%, 20%, 25%, or 29%, up to as much as 75%, 80%, 85%, 90%, 95%, by weight, of the dimer form and generally less than 70%, 65%, 63%, 62%, 61% or 60%, or less of the monomer form. Water-soluble vitamin E derivative mixtures (compositions) include, for example, polyalkylene glycol derivatives of tocopherol, e.g., polyethylene glycol (PEG) derivatives of tocopherol, and polyalkylene glycol derivatives of tocotrienol, e.g., polyethylene glycol (PEG) derivatives of tocotrienol. The water-soluble vitamin E derivatives can include, for example, polyalkylene glycol derivatives of vitamin E, such as polyethylene glycol derivatives of vitamin E, e.g., vitamin E TPGS (D-α-tocopheryl polyethylene glycol succinate), TPGS analogs, TPGS homologs and TPGS derivatives.

As used herein, “tocopherol” and “tocotrienol” refer to any naturally occurring or synthetic form of vitamin E, and can refer to a single compound or a mixture of tocopherols and tocotrienols. Examples of tocopherols include, for example, α-tocopherol, D-α-tocopherol, β-tocopherol, γ-tocopherol and δ-tocopherol. Examples of tocotrienols include, for example, α-tocotrienol, β-tocotrienol, γ-tocotrienol and δ-tocotrienol.

As used herein, a “PEG derivative of vitamin E” or “vitamin E-PEG conjugate” or “vitamin E-PEG derivative,” is a compound containing one or more vitamin E moieties (e.g., a tocopherol or tocotrienol) joined by a covalent bond, for example, an ester, ether, amide or thioester bond, to one or more polyethylene glycol (PEG) moieties, via a linker, such as a dicarboxylic or tricarboxylic acid. Exemplary of PEG derivatives of vitamin E are D-α-tocopheryl polyethylene glycol succinate (TPGS), TPGS analogs, TPGS homologs and TPGS derivatives.

As used herein, “tocopheryl polyethylene glycol succinate,” “TPGS,” “tocopheryl polyethylene glycol succinate surfactant” and “TPGS surfactant” refer to tocopheryl polyethylene glycol conjugates that are formed by covalently joining tocopherol succinate, an ester formed through esterification of tocopherol and succinic acid, to a polyethylene glycol (PEG) moiety via an esterification reaction. The PEG moiety of the TPGS surfactant can be any PEG moiety, for example, a PEG moiety with a molecular weight of between or between about 200 Da to 20,000 Da or about 20,000 Da, for example, PEG moieties having a molecular weight of or about 200, 300, 400, 500, 600, 800, 1000, 3000, 5000, 6000, 8000, 10,000, 20,000 Da, or more; or PEG analogs, including, for example, PEG-NHS (N-hydroxysuccinimide), PEG-aldehyde, PEG-SH, PEG-NH₂, PEG-CO₂H, and branched PEGs.

Exemplary of a TPGS surfactant is TPGS-1000, which has a PEG moiety of 1000 Da. The TPGS can be any natural, water-soluble, tocopherol polyethylene glycol succinate, for example, the food grade TPGS sold under the name Eastman Vitamin E TPGS®, food grade, by Eastman Chemical Company, Kingsport, Tenn. This TPGS is water-soluble form of natural-source vitamin E, which is prepared by esterifying the carboxyl group of crystalline d-alpha-tocopheryl acid succinate with polyethylene glycol 1000 (PEG 1000), and contains between 260 and 300 mg/g total tocopherol. TPGS typically has a reported HLB value of between 12 or 13 or about 12 or 13 and 18 or about 18.

As used herein, “analog” refers to a chemical compound that is structurally similar to another compound (referred to as a parent compound), but differs slightly in composition, for example, due to the variation, addition or removal of an atom, one or more units (e.g., methylene units, —(CH₂)ₙ—) or one or more functional groups. The analog can have different chemical or physical properties compared with the original compound and/or can have improved biological and/or chemical activity. Alternatively, the analog can have similar or identical chemical or physical properties compared with the original compound and/or can have similar or identical biological and/or chemical activity. For example, the analog can be more hydrophilic or it can have altered reactivity as compared to the parent compound. The analog can mimic the chemical and/or biological activity of the parent compound (i.e., it can have similar or identical activity), or, in some cases, can have increased or decreased activity. The analog can be a naturally or non-naturally occurring (e.g., synthetic) variant of the original compound. Other types of analogs include isomers (e.g., enantiomers, diastereomers) and other types of chiral variants of a compound, as well as structural isomers. The analog can be a branched or cyclic variant of a linear compound. For example, a linear compound can have an analog that is branched or otherwise substituted to impart certain advantageous properties (e.g., improved hydrophobicity or bioavailability). Exemplary of the analogs used in the provided compositions and methods are TPGS analogs, which can be formed using the methods provided herein and can be used in place of TPGS in the provided compositions.

As used herein, “tocopheryl polyethylene glycol succinate analog” or “TPGS analog” refers to compounds, other than TPGS, that are similar to a parent TPGS compound, but differ slightly in composition, for example, by the variation, addition or removal of an atom, one or more units (e.g., methylene units, —(CH₂)ₙ—), or one or more functional groups. TPGS analogs include vitamin E-derived surfactants, e.g., tocopheryls and tocotrienols, including PEG derivatives of vitamin E, including vitamin E PEG monomers and dimers, such as, but not limited to, tocopheryl polyethylene glycol sebacate (PTS), tocopheryl polyethylene glycol dodecanodioate (PTD), tocopheryl polyethylene glycol suberate (PTSr), tocopheryl polyethylene glycol azelaate (PTAz), and polyoxyethanyl tocotrienyl sebacate (PTrienS), as well as other PEG derivatives of vitamin E. The compositions provided herein include at least 13%, typically more than 29%, such as 29%-55% or 30%-52%, dimer form in the composition, with the rest of the composition the monomer form or small amounts of other forms and trace contaminants.

Exemplary of TPGS analogs are compounds having the formula shown in Formula I:

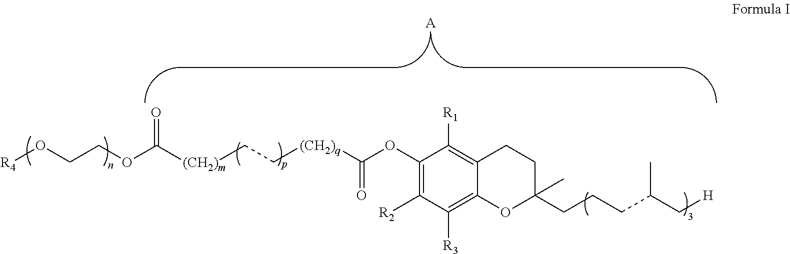

where R₁, R₂ and R₃ each independently is hydrogen (H) or methyl (CH₃); R₄ is H, CH₃ or the portion marked “A”; each dashed line (- - - - -) is independently a single or double bond; n is an integer from 1 to 5000; m and q each independently are 0 or 1; and p is an integer from 1 to 20.

As used herein, “TPGS 1000 analogs” are compounds other than TPGS 1000 that are similar to a parent TPGS 1000 compound due to the addition or removal of an atom, one or more units (e.g., methylene units —(CH₂)ₙ—), or one or more functional groups. TPGS 1000 analogs include, but are not limited to, TPGS compounds having one or more PEG moieties that vary in chain length and molecular weight compared to TPGS 1000, including, for example, TPGS compounds having PEG moieties having a molecular weight between or about between 200 Da to 20,000 Da or about 20,000 Da, for example, PEG moieties having a molecular weight of or about 200, 300, 400, 500, 600, 800, 1000, 3000, 5000, 6000, 8000, 10,000, 20,000 Da, or more. Also exemplary of TPGS 1000 analogs are TPGS compounds including PEG analogs, e.g., PEG-NHS, PEG-aldehyde, PEG-SH, PEG-NH₂, PEG-CO₂H, and branched PEGs. Also exemplary of TPGS 1000 analogs are any TPGS analogs, e.g., vitamin E-derived surfactants, including PEG derivatives of vitamin E, including, but not limited to, tocopheryl polyethylene glycol sebacate (PTS), tocopheryl polyethylene glycol dodecanodioate (PTD), tocopheryl polyethylene glycol suberate (PTSr), tocopheryl polyethylene glycol azelaate (PTAz) and polyoxyethanyl tocotrienyl sebacate (PTrienS), as well as other PEG derivatives of vitamin E.

As used herein, “homolog” refers to an analog that differs from the parent compound only by the presence or absence of a simple unit, such as a methylene unit, or some multiple of such units, e.g., —(CH₂)ₙ—. Typically, a homolog has similar chemical and physical properties as the parent compound. Exemplary of the homologs used in the provided compositions and methods are TPGS homologs.

As used herein, “TPGS homologs” are analogs of TPGS that differ from a TPGS parent compound only by the presence or absence of a simple unit, such as a methylene unit, or some multiple of such units, e.g., —(CH₂)ₙ—. Typically, suitable TPGS homologs have similar surfactant properties compared to the parent compound (TPGS), for example, similar HLB values, for example, HLB values between 12 or about 12 and 20 or about 20. Exemplary of TPGS homologs are tocopheryl polyethylene glycol sebacate (PTS), tocopheryl polyethylene glycol dodecanodioate (PTD), tocopheryl polyethylene glycol suberate (PTSr), tocopheryl polyethylene glycol azelaate (PTAz). Exemplary of TPGS homologs are compounds having the formula in Formula I (above), where neither of the dashed lines represent a double bond and where, when m and q both are 0, and p is greater than 1.

As used herein, “TPGS 1000 homologs” are analogs of TPGS 1000 that differ from a TPGS 1000 parent compound only by the presence or absence of a simple unit, such as a methylene unit, or some multiple of such units, e.g., —(CH₂)ₙ—. Suitable TPGS 1000 homologs have similar surfactant properties compared to the parent compound (TPGS 1000), for example, similar HLB values, for example, HLB values between 12 or about 12 and 20 or about 20, such as 13-18. TPGS 1000 homologs include TPGS 1000 homologs with slight variations in the length of the PEG chain moiety.

As used herein, a “concentrate,” particularly a “pre-gel concentrate” is a composition that generally is formulated for dilution, rather than direct ingestion, or for direct ingestion in a small quantity, such as in a capsule. For purposes herein, a “pre-gel concentrate,” refers to a composition that is formulated as a composition for dilution, but also is provided in capsules, such as soft gels, for direct ingestion, where a single capsule provides a single dosage or a fractional dosage.

As used herein, a “pre-gel concentrate” refers to a composition that contains a particular volume to be encapsulated, contains a single dosage or fractional dosage of a composition containing non-polar ingredients.

As used herein, “fractional dosage” refers to an amount that is less that a full dosage so that, when provided as a capsule, a plurality of capsules will be required to provide a single dosage. Typically, a fractional dosage is at least 20%, 25%, 50% of a full dosage.

As used herein, “colloid” refers to a mixture containing two phases, a dispersed phase and a continuous phase, with the dispersed phase containing particles (droplets) distributed throughout the continuous phase. Colloidal mixtures include aerosols, foams, and dispersions, for example, emulsions, for example, nanoemulsions. A liquid colloid, for example, a nanoemulsion, can have a similar appearance, for example, similar clarity, to a solution in which there is no dispersed phase.

As used herein, “emulsion” refers to a colloidal dispersion of two immiscible liquids, for example, an oil and water (or other aqueous liquid, e.g., a polar solvent), one of which is part of a continuous phase and the other of which is part of a dispersed phase. Emulsions typically are stabilized by one or more surfactants and/or co-surfactants and/or emulsion stabilizers. Surfactants form an interfacial film between the oil and water phase of the emulsion, providing stability. Typically, emulsions contain micelles that contain one or more surfactants surrounding a non-polar compound which is dispersed in the water phase. In general, emulsions (e.g., oil-in-water emulsions) are colloidal dispersions of two immiscible liquids (e.g., oil and an aqueous liquid, such as water) that contain a continuous and a dispersed phase. Emulsions can be used to disperse non-polar compounds in aqueous liquids. In an oil-in-water emulsion, the dispersed phase is an oil phase and the continuous phase is an aqueous (e.g., water) phase.

As used herein, “surfactant” refers to synthetic and naturally occurring amphiphilic molecules that have hydrophobic portion(s) and hydrophilic portion(s). Due to their amphiphilic (amphipathic) nature, surfactants typically can reduce the surface tension between two immiscible liquids, for example, the oil and water phases in an emulsion, stabilizing the emulsion. Surfactants can be characterized based on their relative hydrophobicity and/or hydrophilicity. For example, relatively lipophilic surfactants are more soluble in fats, oils and waxes, and typically have HLB values less than or about 10, while relatively hydrophilic surfactants are more soluble in aqueous compositions, for example, water, and typically have HLB values greater than or about 10. Relatively amphiphilic surfactants are soluble in oil- and water-based liquids and typically have HLB values close to 10 or about 10.

As used herein, “co-surfactant” is used to refer to a surfactant that is used in the provided compositions in combination with the primary surfactant, for example, the water-soluble vitamin E derivative mixtures (compositions) described herein, for example, to improve the emulsification of the provided compositions and/or compounds, for example, to emulsify the ingredients. In one example, the provided compositions can contain at least one surfactant and at least one co-surfactant. Typically, the co-surfactant represents a lower percent, by weight (w/w), of the provided compositions, compared to the surfactant. Thus, the provided compositions typically have a lower concentration of the co-surfactant(s) than of the surfactant.

As used herein, “HLB” refers to a value that is used to index and describe a surfactant according to its relative hydrophobicity/hydrophilicity, relative to other surfactants. A surfactant's HLB value is an indication of the molecular balance of the hydrophobic and hydrophilic portions of the surfactant, which is an amphipathic molecule. Each surfactant and mixture of surfactants (and/or co-surfactants) has an HLB value that is a numerical representation of the relative weight percent of hydrophobic and hydrophilic portions of the surfactant molecule(s). HLB values are derived from a semi-empirical formula. The relative weight percentages of the hydrophobic and hydrophilic groups are indicative of surfactant properties, including the molecular structure, for example, the types of aggregates the surfactants form and the solubility of the surfactant. See, for example, Griffin (1949) J. Soc. Cos. Chem. 1:311. Surfactant HLB values range from 1-45, while the range for non-ionic surfactants typically is from 1-20. The more lipophilic a surfactant is, the lower its HLB value. Conversely, the more hydrophilic a surfactant is, the higher its HLB value.

As used herein, “micelle” refers to aggregates formed by surfactants that typically form when a surfactant is present in an aqueous composition, typically when the surfactant is used at a concentration above the critical micelle concentration (CMC). In micelles, the hydrophilic portions of the surfactant molecules contact the aqueous or the water phase, while the hydrophobic portions form the core of the micelle, which can encapsulate non-polar ingredient(s), for example, the non-polar ingredients in the provided concentrates. Typically, the surfactants in the provided concentrates form micelles containing the non-polar ingredient at their center in the aqueous liquid dilution compositions. Typically, the micelles in the provided concentrates have a particle size of about 1000 nm, typically less than or less than about 500 nm, typically less than 300 or less than about 300 nm, for example, less than 250 nm or less than about 250 nm, for example, less than 200 nm or less than about 200 nm, for example, less than or less than about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nm.

As used herein, “shelf life” refers to a time period within which the provided compositions retain desirable organoleptic properties, for example, the ability of the provided compositions to retain desirable organoleptic properties for a period of time, for example, for at least or more than 1, 2, 3, 4, or more weeks, typically at least or more than 1, 2, 3, 4, 5, 6, 7, 8, 9, 10, 11, 12, or more months, or at least or more than 1, 2, 3, 4, or more years. In one example, the compositions retain desirable organoleptic properties if they exhibit one or more of these described characteristics, over time, when kept at a particular temperature. In one example, the compositions retain desirable organoleptic properties at room temperature, for example, 25° C. or about 25° C. In another example, the compositions retain desirable organoleptic properties at between 19° C. and 25° C. In another example, the compositions retain desirable organoleptic properties at refrigerated temperatures, for example, 4° C. or about 4° C., or at frozen temperatures, for example, at −20° C. or about −20° C. In another example, the compositions retain desirable organoleptic properties at elevated temperatures, for example, at 40° C. or at about 40° C.

As used herein, “stability” of compositions, such as the pre-gel emulsions and concentrates and soft gels to remain free from one or more deleterious changes over a period of time, for example, at least or longer than 1 day, 1 week, 1 month, 1 year, or more. For example, a concentrate or soft gel can be described as stable if it is formulated such that it remains free from oxidation or substantial oxidation over time, remains clear over time, remains safe and/or desirable for human consumption over time, does not form precipitates and remains free of microbial or other contamination.

As used herein, “room temperature” and “ambient temperature” are used to describe a temperature that is common in one or more enclosed spaces in which human beings typically are or reside. Room temperature can vary, but generally refers to temperatures between or between about 19° C. and 25° C. When a composition is stored at room temperature, it should be understood it is generally kept at a temperature within this range or about within this range.

As used herein, “refrigerated temperature” refers to a temperature that is common in a refrigerator, for example, a household or restaurant refrigerator, for example, a temperature that is cooler than room temperature, but typically a few degrees above the freezing point of water. Typically, refrigerated temperatures are between or between about 0° C. and 10° C., for example, at or about 4° C. When a composition is stored at a refrigerated temperature, it should be understood that it is kept at a temperature common to household or industrial refrigerators.

As used herein, “hydrophilic” and “polar” refer synonymously to ingredients and/or compounds having greater solubility in aqueous liquids, for example, water, than in fats, oils and/or organic solvents (e.g., methanol, ethanol, ethyl ether, acetone and benzene).

As used herein, a “solvent” is an ingredient that can be used to dissolve another ingredient. Solvents include polar and non-polar solvents. Non-polar solvents include oils and other non-polar ingredients that dissolve non-polar compounds and ingredients. Typically, the non-polar solvent is an oil that is included in the concentrates or liquid dilution compositions provided herein in addition to the non-polar ingredient. The non-polar solvent typically is not the non-polar ingredient itself, i.e., is distinct from the non-polar ingredient. More than one non-polar solvent can be used. Certain compounds, for example, flaxseed oil and safflower oil, can be non-polar solvents and non-polar ingredients. Typically, the non-polar solvent contains one or more oils, typically oils other than the non-polar ingredient or oil(s) not contained in the non-polar ingredient. Exemplary non-polar solvents include, but are not limited to, oils (in addition to the non-polar ingredient), for example, vitamin E oil, flaxseed oil, CLA, borage oil, rice bran oil, d-limonene, canola oil, corn oil, MCT oil, and oat oil. Other oils also can be used.

As used herein, “polar solvent” refers to a solvent that is readily miscible with water and other polar solvents. Polar solvents are well-known and can be assessed by measuring any parameter known to those of skill in the art, including dielectric constant, polarity index and dipole moment (see, e.g., Przybitek (1980) “High Purity Solvent Guide,” Burdick and Jackson Laboratories, Inc.). For example, polar solvents generally have high dielectric constants, such as greater than or about 15, generally have high polarity indices, typically greater than or about 3, and generally large dipole moments, for example, greater than or about 1.4 Debye.

As used herein, “non-polar,” “lipophilic” and “lipid-soluble” synonymously refer to compounds and/or ingredients, for example, non-polar compounds and non-polar ingredients, which have greater solubility in organic solvents (e.g., ethanol, methanol, ethyl ether, acetone and benzene), fats and oils than in aqueous liquids, for example, water. Non-polar ingredients include drugs, hormones, vitamins, nutrients and other lipophilic compounds. Typically, non-polar ingredients are poorly water-soluble, for example, water insoluble or compounds having low water solubility. Exemplary non-polar ingredients include ingredients that contain one or more non-polar compounds, for example, lipid-soluble drugs, hormones, essential fatty acids, for example, polyunsaturated fatty acids (PUFA), for example, omega-3 and omega-6 fatty acids, vitamins, nutrients, nutraceuticals, minerals and other compounds. Additional exemplary non-polar ingredients are described herein. The provided compositions can be formulated with any non-polar ingredient, for example, any non-polar ingredient that is or contains a non-polar compound.

As used herein, “non-polar ingredient” refers to a component that is a non-polar compound or contains one or more non-polar compounds. For example, the non-polar ingredient algae oil contains polyunsaturated fatty acid non-polar compounds, for example, the omega-3 polyunsaturated fatty acid DHA.

As used herein, “non-polar compound” refers to a compound that contains an active component or is active such that, when administered to a subject, for example, a human, induces or is proposed to induce a desired response, such as altering body function at the cellular, tissue, organ or other level, and/or altering the cosmetic appearance or other property, or a compound that is ingested in order to achieve a desired effect. Non-polar compounds include any synthetic or natural non-polar ingredient or compound, including a pharmaceutical, drug, therapeutic, nutritional supplement, herb, hormone or other ingredient. Non-polar compounds can include the non-polar compounds listed herein, as well as other pharmaceutically acceptable or food-grade active derivatives of the non-polar compounds, for example, salts, esters, amides, prodrugs, active metabolites, isomers, fragments and analogs. Non-polar compounds can include compounds proven to have a desired effect and also compounds thought to produce such effects, for example, compounds typically ingested for nutritional supplementation purposes. The non-polar compound can be contained in a non-polar ingredient or is the non-polar ingredient.

As used herein, a “subject” includes an animal, typically a mammal, typically a human.

As used herein, an “additive” includes anything that one can add to a food, beverage, or other human consumable to enhance one or more of its nutritional, pharmaceutical, dietary, health, nutraceutical, health benefit, energy-providing, treating, holistic, or other properties. For example, the additives can be oil-based additives (e.g., non-polar compounds), such as nutraceuticals; pharmaceuticals; vitamins, for example, oil-soluble vitamins, e.g., vitamin D, vitamin E and vitamin A; minerals; fatty acids, such as essential fatty acids, for example, polyunsaturated fatty acids, e.g., omega-3 fatty acids and omega-6 fatty acids, such as alpha-linolenic acid (ALA), docosahexaenoic acid (DHA), eicosapentaenoic acid (EPA), gamma-linolenic acid (GLA), CLA, saw palmetto extract, flaxseed oil, fish oil and algae oil. phytosterols; coenzymes, such as coenzyme Q10; and any other oil-based additives.

As used herein, “water insoluble” refers to a compound that does not dissolve when the compound is mixed with water, for example, when mixed with water at room temperature, for example, between or between about 25° C. and 50° C.

As used herein, “low water solubility” refers to a compound that has a solubility in water of less than or about 30 mg/mL, for example, when mixed with water at room temperature, such as between or between about 25° C. and 50° C. As used herein, “poorly water-soluble” can be used to refer to compounds, for example, non-polar compounds, that are water insoluble or have low water solubility.

As used herein, “waxy” is used to describe compositions and materials, typically oil-soluble compositions or materials, that are similar in consistency to one or more waxes or semi-solids. Typically, waxy compositions are of relatively low viscosity a little above their liquefying point. Exemplary of waxes that have waxy consistencies are natural waxes, including waxes of vegetal origin, such as purcellin, shea butter, cocoa butter, Japan wax, esparto grass wax, cork wax, Guaruma wax, rice shoot wax, Ouricury wax, montan wax, sunflower wax, sugar cane wax, carnauba wax, candelilla wax, lanolin; fruit-derived waxes, such as orange wax, lemon wax, grapefruit wax and bayberry wax, and the like; waxes of animal origin, such as beeswax, woolwax, spermateci and bear fat, shellac wax, and the like; mineral waxes such as ceresine and ozokerite waxes; and synthetic waxes, including petroleum-based waxes such as paraffin, petrolatum, micro wax, polyalkylene and polyethyleneglycol waxes, e.g. polyethylene wax; waxes based on chlorinated naphthalenes, such as “Halowax” and synthetic hydrocarbon waxes.

As used herein, “food and beverage product” refers to a product that is suitable for human consumption. For example, “food and beverage product” can refer to a pre-emulsion concentrate that is dissolved in a solvent, typically an aqueous solvent, e.g., water, to form a beverage composition or beverage product. “Food and beverage product” can also refer to the final product that is suitable for human consumption, such as the soft gel compositions.

As used herein, “fatty acid” refers to straight-chain hydrocarbon molecules with a carboxyl (—COOH) group at one end of the chain.

As used herein, “polyunsaturated fatty acid” and “PUFA” are used synonymously to refer to fatty acids that contain more than one carbon-carbon double bonds in the carbon chain of the fatty acid. PUFAs, particularly essential fatty acids, are useful as dietary supplements.

As used herein, “essential fatty acids” are PUFAs that mammals, including humans, cannot synthesize using any known chemical pathway. Thus, essential fatty acids must be obtained from diet or by supplementation. Exemplary of essential PUFA fatty acids are the omega-3 (ω3; n-3) fatty acids and omega-6 (ω-6; n-6) fatty acids.

As used herein, “omega-3 (ω-3; n-3) fatty acids” and “omega-3 fatty acids” are used synonymously to describe methylene-interrupted polyenes which have two or more cis double bonds separated by a single methylene group, in which the first double bond appears at the third carbon from the last (w) carbon. Omega-3 fatty acids are used as dietary supplements, for example, for disease treatment and prevention. The provided concentrates and liquid dilution compositions can contain non-polar active ingredients that include at least one omega-3 fatty acid. Exemplary of omega-3 fatty acids are alpha-linolenic acid (α-linolenic acid; ALA) (18:3 ω-3) (a short-chain fatty acid); stearidonic acid (18:4 ω-3) (a short-chain fatty acid); eicosapentaenoic acid (EPA) (20:5 ω-3); docosahexaenoic acid (DHA) (22:6 ω-3); eicosatetraenoic acid (24:4 ω-3); docosapentaenoic acid (DPA, clupanodonic acid) (22:5 ω-3); 16:3 ω-3; 24:5 ω-3 and nisinic acid (24:6 ω-3). Longer chain omega-3 fatty acids can be synthesized from ALA (the short-chain omega-3 fatty acid). Exemplary of non-polar ingredients containing omega-3 fatty acids are non-polar ingredients containing DHA and/or EPA, for example, containing fish oil, krill oil and/or algae oil, for example, microalgae oil, and non-polar ingredients containing alpha-linolenic acid (ALA), for example, containing flaxseed oil.

As used herein, “omega-6 (ω-6; n-6) fatty acids” and “omega-6 fatty acids” are used synonymously to describe methylene-interrupted polyenes which have two or more cis double bonds separated by a single methylene group, in which the first double bond appears at the sixth carbon from the last (ω) carbon. The provided concentrates and soft gel compositions can contain non-polar ingredients that include at least one omega-6 fatty acid. Exemplary of omega-6 fatty acids are linoleic acid (18:2 ω-6) (a short-chain fatty acid); gamma-linolenic acid (GLA) (18:3 ω-6); dihomo gamma linolenic acid (DGLA) (20:3 ω-6); eicosadienoic acid (20:2 ω-6); arachidonic acid (AA) (20:4 ω-6); docosadienoic acid (22:2 ω-6); adrenic acid (22:4 ω-6); and docosapentaenoic acid (22:5 ω-6). Exemplary of non-polar active ingredients containing omega-6 fatty acids are ingredients containing GLA, for example, borage oil. Also exemplary of omega-6-containing non-polar ingredients are ingredients containing conjugated fatty acids, for example, conjugated linoleic acid (CLA) and ingredients containing saw palmetto extract.

As used herein, “algae oil” refers to any oil derived from marine dinoflagellates in, for example, microalgae, for example, Crypthecodinium sp, particularly, Crypthecodinium cohnii. Algae oil can be used as a non-polar ingredient in the provided concentrates and soft gel compositions. The algae oil typically contains DHA. The algae oil can be a source of EPA.

As used herein, “fish oil” refers to any oil derived from any fish, typically a cold water fish, for example, from fish tissue, such as from frozen fish tissue, for example, from cod liver. Fish oil can be used as a non-polar ingredient in the provided concentrates and soft gels. The fish oil typically contains DHA. The fish oil can also contain EPA. For example, the fish oil can contain a mixture of DHA and EPA.

As used herein, “preservative” refer to ingredients that prevent or reduce microbial contamination, and/or in general improve the shelf life of a composition provided herein. Preservatives, particularly food and beverage preservatives, are well known. A preservative is selected that is safe for human consumption, for example, in foods and beverages, for example, a GRAS certified and/or Kosher-certified preservative.

As used herein, “flavor” is any ingredient that changes, typically improves, the taste and/or smell of the provided compositions.

As used herein, “G.R.A.S.” and “GRAS” are used synonymously to refer to compounds, compositions and ingredients that are “Generally Regarded as Safe” by the USDA and FDA for use as additives, for example, in foods, beverages and/or other substance for human consumption, such as any substance that meets the criteria of sections 201(s) and 409 of the U.S. Federal Food, Drug and Cosmetic Act. Typically, the concentrates and soft gel compositions provided herein are GRAS certified.

As used herein, “kosher” is used to refer to substances that conform to Jewish Kosher dietary laws, for example, substances that do not contain ingredients derived from non-kosher animals or do not contain ingredients that were not made following kosher procedures. Typically, the concentrates and soft gel compositions provided herein are Kosher-certified.

As used herein, “rapid cooling” refers to a process by which a composition is cooled to a desired temperature, for example, between or between about 25° C. and 45° C., in less than or less than about 2 hours, typically less than or less than about 1 hour, for example, less than or less than about 30 minutes, such as 15 minutes.

As used herein, “w/w,” “by weight,” “% by weight,” “wt %” and “weight percent” are used synonymously to express the ratio of the mass of one component of a composition compared to the mass of the entire composition. For example, when the amount of a particular ingredient represents 1%, by weight (w/w), of a concentrate, the mass of that ingredient is 1% of the mass of the entire concentrate. Similarly, when the amount of an ingredient is 50% (w/w) of the concentrate, the mass of that ingredient is 50% of the entire mass of the concentrate. Similarly, when a composition and/or a compound contains 10%, by weight, of an ingredient, the mass of the ingredient is 10% of the total mass of the composition or compound. When a composition contains 10 wt % of an ingredient, the mass of that ingredient is 10% of the mass of the entire composition. When only a concentration, amount, or percentage (without units) is listed, it is to be understood that the concentration or percentage is a concentration or percentage by weight.

As used herein “v/v” and “volume percent” are used synonymously to express the ratio of the volume of one component of a composition to the volume of the entire composition.

As used herein, “not more than” and “NMT” refer to a quantity that is less than or equal to the listed quantity. Similarly, “not less than” and “NLT” refer to a quantity that is greater than or equal to the listed quantity.

As used herein, the singular forms “a,” “an” and “the” include plural referents unless the context clearly dictates otherwise. Thus, for example, reference to a composition containing “a non-polar ingredient” includes compositions with one or more non-polar ingredients.

As used herein, ranges and amounts can be expressed as “about” a particular value or range. About also includes the exact amount. Hence, “about 5 grams” means “about 5 grams” and also “5 grams.” It also is understood that ranges expressed herein include whole numbers within the ranges and fractions thereof. For example, a range of between 5 grams and 20 grams includes whole number values such as 5, 6, 7, 8, 9, 10, 11, 12, 13, 14, 15, 16, 17, 18, 19 and 20 grams, and fractions within the range including, but not limited to, 5.25, 6.72, 8.5 and 11.95 grams.

As used herein, “optional” or “optionally” means that the subsequently described event or circumstance does or does not occur and that the description includes instances where said event or circumstance occurs and instances where it does not. For example, a reaction mixture that “optionally includes a catalyst” means that the reaction mixture contains a catalyst or it does not contain a catalyst.

As used herein, “consisting essentially of” means containing the following list of ingredient(s), and not including any additional non-polar ingredient other than those listed.

### B. SOFT GEL AND PRE-GEL COMPOSITIONS CONTAINING NON-POLAR INGREDIENTS AND PEG DERIVATIVES OF VITAMIN E

Compositions and methods for formulating compositions containing non-polar compounds and ingredients are provided herein. Provided herein are soft gel compositions containing a non-aqueous pre-gel concentrate in a shell or coating, suitable for human consumption, and methods for preparing the compositions. The soft gel compositions contain a non-aqueous pre-gel composition that contains one or more non-polar ingredients, a surfactant, and a high amount (greater than 2%, 3%, 5%, 6%, 7%, 10%, up to 20%) of non-aqueous solvent. The pre-gel compositions also are provided. By virtue of the ingredients, including the high amount of non-aqueous solvent, the pre-gel compositions exhibit advantageous properties, such as, for example, the formulation of the ingredients results in stable pre-gel compositions that do not form precipitates and exhibit enhanced bioavailability of the non-polar compounds, for example, when consumed.

In particular, the non-aqueous pre-gel compositions, i.e., concentrates, provided herein contain non-polar ingredients that are or contain a non-polar compound and/or mixtures of non-polar compounds, at least one surfactant, and one or more non-aqueous solvents. The pre-gel concentrates are encapsulated by enclosing the pre-gel concentrates in a desired coating or shell to form the soft gel compositions provided herein. The pre-gel concentrates contain from 40% to as much as 90% of the non-polar ingredient, by weight, of the concentrate and between 5% and 20% non-aqueous solvent, and the remainder a surfactant(s), such as a PEG derivative of vitamin E, particularly TPGS. The resulting compositions do not contain precipitated non-polar ingredients. The compositions described herein, in particular, the soft-gel compositions and pre-gel compositions, i.e., concentrate, contain high concentrations of non-polar ingredients and have enhanced bioavailability, for example, after human consumption. Further description and examples of the pre-gel concentrates and soft gel compositions are provided below.

1. Non-Aqueous Pre-Gel Compositions Containing Non-Polar Ingredients

Non-aqueous pre-gel compositions (also referred to as concentrates) that contain non-polar ingredients, a surfactant(s), and one or more non-aqueous solvents are provided. The pre-gel concentrates are formulated by selecting ingredients and concentrations of the ingredients as described herein that yield non-aqueous pre-gel concentrates having one or more desired properties, for example, pre-gel concentrates that do not contain any precipitated non-polar ingredient. The pre-gel concentrates contain high concentrations of the non-polar ingredients ranging from at or about 30% to 90% by weight of the pre-gel concentrate, from or about 5% to about 20% by weight of the non-aqueous solvent, and a PEG derivative of vitamin E, such as TPGS, in an amount that is about or at 5% to 65%, by weight, of the pre-gel concentrate. Additional optional ingredients, such as other surfactants, emulsifiers, and co-solvents, in amounts totaling less than 5% or less than about 5% can be included.

By virtue of the high concentration of the non-polar ingredients, and also the surfactant, the pre-gel concentrates as provided herein can be waxy, having the consistency of a substance such as wax, for example, a lip balm, or a semi-solid at room temperature, for example, at 25° C. or about 25° C., and become liquid at higher temperatures, for example when heated to higher temperatures, such as to 125° F. or about 125° F., or to 50° C. or about 50° C. or to 60° C. or about 60° C., or the concentrates can be liquid. The pre-gel concentrates provided herein can be a liquid at room temperature. Whether the pre-gel concentrate is waxy (i.e., semi-solid) or a liquid depends upon the particular components and amounts thereof.

The pre-gel concentrates are then introduced into or formulated with materials, such as gelatins and/or gelatin replacements and other ingredients to form soft gel capsules. Each capsule is designed for oral administration and typically contains an amount of the pre-gel composition to provide a single dosage or fractional amount of the non-polar ingredients.

a. Non-Polar Ingredients

The non-aqueous pre-gel concentrates provided herein contain one or more non-polar ingredients that are or contain one or more non-polar compounds. Non-polar ingredients include any lipophilic or lipid-soluble compound that has greater solubility in organic solvents (e.g., benzyl alcohol, benzyl benzoate, d-limonene, ethanol, methanol, ethyl ether, acetone, and benzene) and in fats and oils, than in polar solvents, for example, water. Typically, the non-polar ingredients are poorly water-soluble, for example, water insoluble, or are compounds that have low water solubility. The non-polar ingredients include, but are not limited to, drugs, hormones, vitamins, nutrients and other lipophilic compounds. Exemplary non-polar ingredients include, but are not limited to, omega-3 EPA and DHA, resveratrol, sesamin, curcumin, Boswellia (Boswellic acids), lipoic acid, such as alpha lipoic acids, capsaicinoids, PQQ, carotenoids, such as astaxanthin, zeaxanthin, lutein, beta-carotene, and lycopene, and vitamins, such as vitamin A, vitamin D, and vitamin E complexes, vitamin K1 and vitamin K as MK7. Exemplary non-polar ingredients are listed herein below. The non-polar ingredient differs from the surfactant, e.g., polyalkylene glycol derivative of vitamin E, for example, the non-polar ingredient is not a polyalkylene glycol vitamin E derivative.

The non-polar ingredients are included in the pre-gel concentrate in an amount from about or at 30% to 90%, typically 35% to 85% or 40 to 90%, inclusive, or 45% to 90%, 40% to 85%, or 40% to 75%, all inclusive by weight of the composition. The amount included depends upon the particular ingredient and desired dosage. Exemplary of non-polar ingredients that can be used in the provided pre-gel concentrates include, but are not limited to:

non-polar ingredients containing essential fatty acids, such as polyunsaturated fatty acids (PUFAs), for example, gamma-linolenic acid (GLA), e.g., borage oil and evening primrose (Oenothera biennis) oil, blackcurrant seed oil, hemp seed oil and spirulina extract; ingredients containing omega-3 fatty acids, such as natural and synthetic omega-3 fatty acids, for example, ingredients containing omega-3 polyunsaturated long-chain fatty acids, including eicosapentaenoic acid (EPA) (20:5ω3); docosahexaenoic acid (DHA) (22:6ω3); eicosatetraenoic acid (24:4ω3); docosapentaenoic acid (DPA, clupanodonic acid) (22:5ω3); 16:3ω3; 24:5ω3 and/or nisinic acid (24:6ω3), e.g., fish oil, algae oil, krill oil, canola oil, flaxseed oil, soybean oil and walnut oil; ingredients containing short-chain omega-3 fatty acids, for example, alpha-linolenic acid (α-linolenic acid; ALA; 18:3ω3) and stearidonic acid (18:4ω3), esters of an omega-3 fatty acid and glycerol, for example, monoglycerides, diglycerides and triglycerides, esters of an omega-3 fatty acid and a primary alcohol, for example, fatty acid methyl esters and fatty acid esters, precursors of omega-3 fatty acid oils, for example, EPA precursor, DHA precursor, derivatives such as polyglycolized derivatives or polyoxyethylene derivatives, oils containing the omega-3 fatty acids, for example, fish oil (marine oil), e.g., highly purified fish oil concentrates, perilla oil, krill oil, and algae oil, e.g., microalgae oil; ingredients containing omega-6 fatty acids, such as ingredients containing linoleic acid (18:2ω6) (a short-chain fatty acid); gamma-linolenic acid (GLA; 18:3ω6); dihomo gamma linolenic acid (DGLA; 20:3ω6); eicosadienoic acid (20:2ω6); arachidonic acid (AA; 20:4ω6); docosadienoic acid (22:2ω6); adrenic acid (22:4ω6); and/or docosapentaenoic acid (22:5ω6), for example, borage oil, corn oil, cottonseed oil, grapeseed oil, peanut oil, primrose oil, e.g., evening primrose (Oenothera biennis) oil, blackcurrant seed oil, hemp seed oil, spirulina extract, safflower oil, sesame oil, coconut oil and soybean oil;

ingredients containing non-essential fatty acids, such as ingredients containing omega-5 fatty acids, such as natural and synthetic omega-5 fatty acids, for example, ingredients containing punicic acid (18:3ω-5) and 9-tetradecenoic acid (myristoleic acid), and/or esters of omega-5 fatty acids, such as ingredients containing cetyl esters of omega-5 fatty acids, e.g., cetyl myristoleate; omega-7 fatty acids, such as vaccenic acid (18:1 trans-11) and palmitoleic acid (9-hexadecenoic acid); and omega-9 fatty acids, such as oleic acid (18:1ω9), eicosatrienoic acid (20:3ω-3), and erucic acid (22:1ω9); other fatty acids, such as triglycerides, including medium chain triglycerides and long chain triglycerides and mixtures thereof; polar lipids, for example, lipids of ethers, phosphoric acid, choline, fatty acids, glycerol, glycolipids, and phospholipids (e.g., phosphatidylcholine (lecithin), phosphatidylethanolamine, and phosphatidylinositol); saw palmetto extract; ethyl linoleate; herb oils, for example, garlic oils and scordinin; short-chain saturated fatty acids (4:0-10:0); lauric acid (12:0); myristic acid (14:0); pentadecanoic acid (15:0); palmitic acid (16:0); palmitoleic acid (16:1 ω7); heptadecanoic acid (17:0); stearic acid (18:0); and arachidic acid (20:0);

micronutrients, such as vitamins, minerals, co-factors, for example, coenzyme Q10 (coQ10, also called ubiquinone), ubiquinol, turmeric extract (curcuminoids), saw palmetto lipid extract (saw palmetto oil), echinacea extract, hawthorn berry extract, ginseng extract, lipoic acid (thioctic acid), such as alpha-lipoic acid, ascorbyl palmitate, kava extract, St. John's Wort (hypericum, Klamath weed, goat weed), extract of quercitin, dihydroepiandrosterone, pyrroloquinoline quinone (PQQ), Boswellia serrata extract, and indol-3-carbinol;

carotenoids, including hydrocarbons and oxygenated, alcoholic derivatives of hydrocarbons, for example, beta carotene, mixed carotenoid complex, lutein, lycopene, zeaxanthin, cryptoxanthin, for example, beta-crytoxanthin, beta carotene, astaxanthin, bixin, canthaxanthin, capsanthin, capsorubin, apo-carotenal, beta-12′-apo-carotenal, “Carotene” (mixture of alpha- and beta-carotene), gamma carotene, ciolerythrin, and esters of hydroxyl- or carboxyl-containing members thereof;

fat-soluble vitamins and ingredients containing fat-soluble vitamins, for example, any of vitamins A, D, E and K, and corresponding pro-vitamins and vitamin derivatives, such as esters, with an action resembling that of any of vitamins A, D, E or K, for example, retinol (vitamin A) and pharmaceutically acceptable derivatives thereof, such as palmitate ester of retinol and other esters of retinol, calciferol (vitamin D) and its pharmaceutically acceptable derivatives thereof and precursors of vitamin D, d-alpha tocopherol (vitamin E) and derivatives thereof, including pharmaceutical derivatives thereof, for example, tocotrienols, d-alpha tocopherol acetate and other esters of d-alpha tocopherol, and ascorbyl palmitate, a fat-soluble version of vitamin C;

phytochemicals, including phytoestrogens, for example, genistein and daidzein, such as isoflavones, e.g., soy isoflavones, flavonoids, phytoalexins, for example, resveratrol (3,5,4′-trihydroxystilbene), red clover extract, and phytosterols;

lipid-soluble drugs, including natural and synthetic forms of immunosuppressive drugs, such as cyclosporin, protease inhibitors such as ritonavir, macrolide antibiotics and oil soluble anesthetics such as propofol, natural and synthetic forms of steroidal hormones, for example, estrogens, estradiols, progesterone, testosterone, cortisone, phytoestrogens, dehydroepiandrosterone (DHEA), growth hormones and other hormones; and oil-soluble acids and alcohols, for example, tartaric acid, lactylic acid, butylated hydroxyanisole, butylated hydroxytoluene, lignin, sterols, polyphenolic compounds, oryzanol, cholesterol, phytosterols, flavonoids, such as quercetin and resveratrol, and diallyl disulfides;

cannabinoids, including natural, synthetic, and semi-synthetic cannabinoids, for example, phytocannabinoids, endocannabinoids, and synthetic cannabinoids; and

hops-containing ingredients, including ingredients isolated or derived from hops (Humulus lupulus L.), such as extracts of hops cones, for example, hops oils, hops resins or hops resin derivatives, hops acids or hops acid derivatives, or mixtures thereof.

1) Fatty Acids

Exemplary of the non-polar ingredients contained in the pre-gel concentrates provided herein are ingredients containing fatty acids, for example, essential fatty acids and non-essential fatty acids. Fatty acids are straight-chain hydrocarbon molecules with a carboxyl (COOH) group at one end of the chain. PUFAs are fatty acids that contain more than one carbon-carbon double bond in the carbon chain of the fatty acid. PUFAs, particularly essential fatty acids, are useful as dietary supplements.

Different nomenclature is used to describe fatty acid molecules. Lipid nomenclature, for example, 18:3 ω-3, indicates the carbon chain length, number of double bonds and the position along the carbon chain of the first carbon-carbon double bond in a fatty acid. Using this nomenclature, each carbon along the chain is labeled according to its position relative to one end of the chain. For example, the first carbon away from the carboxylate end is named α, the second is named β, and so forth. The last carbon in the molecule (furthest from the carboxy group) always is labeled w (or omega, or n). The number of carbons and the number of double bonds are listed first in the lipid name of a fatty acid, separated by a colon. For example, the name “18:3” indicates that the molecule has eighteen (18) carbons and three (3) double bonds. Following these numbers, the position at which the first double bond appears, relative to the last (ω) carbon, is listed. For example, the nomenclature, 18:3 ω-3 (or 18:3 omega-3; or 18:3 n-3), describes a fatty acid with eighteen (18) carbons and three (3) double bonds, the first of which occurs at the third carbon away from the omega carbon.

Alternatively, chemical nomenclature can be used. The chemical name of a fatty acid describes the position of each double bond. In the chemical naming, the carbons are numbered, beginning with 1, starting with the carbon that is part of the carboxy (COOH) group. Thus, with this numbering system, the a carbon is labeled “2.” The chemical name of the fatty acid lists the first carbon (from the COOH end) to participate in each double bond.

a) Polyunsaturated Fatty Acid (PUFA)-Containing Ingredients

Exemplary of the non-polar ingredients contained in the pre-gel concentrates provided herein are non-polar ingredients containing fatty acids, for example, non-polar ingredients containing the non-polar compounds polyunsaturated fatty acids (PUFAs). PUFAs can be essential fatty acids or non-essential fatty acids. Certain PUFAs are called essential fatty acids because they are required for biological processes and mammals, including humans, cannot synthesize them using any known chemical pathway, and therefore must obtain them from diet or by supplementation (U.S. Pat. No. 6,870,077; Covington (2004) Am. Fam. Physician 70(1):133-140). The essential PUFAs are the omega-3 (ω-3; n-3) fatty acids and the omega-6 (ω-6; n-6) fatty acids. Omega-3 and omega-6 fatty acids are methylene interrupted polyenes which have two or more cis double bonds separated by a single methylene group. Exemplary of omega-3 fatty acids are alpha-linolenic acid (α-linolenic acid; ALA; 18:3 ω-3) (a short-chain fatty acid); stearidonic acid (18:4 ω-3) (a short-chain fatty acid); eicosapentaenoic acid (EPA; 20:5 ω-3); docosahexaenoic acid (DHA; 22:6 ω-3); eicosatetraenoic acid (24:4 ω-3); docosapentaenoic acid (DPA; clupanodonic acid; 22:5 ω-3); 16:3 ω-3; 24:5 ω-3 and nisinic acid (24:6 ω-3). Longer chain omega-3 fatty acids can be synthesized from ALA (the short-chain omega-3 fatty acid). Exemplary of omega-6 fatty acids are linoleic acid (18:2 ω-6) (a short-chain fatty acid); gamma-linolenic acid (GLA; 18:3 ω-6); dihomo gamma linolenic acid (DGLA; 20:3 ω-6); eicosadienoic acid (20:2 ω-6); arachidonic acid (AA; 20:4 ω-6); docosadienoic acid (22:2 ω-6); adrenic acid (22:4 ω-6); and docosapentaenoic acid (22:5 ω-6).

While the longer chain omega-3 and omega-6 essential fatty acids can be synthesized from ALA (the short-chain omega-3 fatty acid) and linolenic acid (LA), respectively, evidence suggests that conversion of these short-chain fatty acids in humans is slow. Thus, a major source of long-chain essential PUFAs is dietary (see, e.g., Ross et al. (2007) Lipids Health Dis. 6:21 and Lands (1992) FASEB J. 6(8):2530). Dietary supplements containing PUFAs, particularly essential PUFAs, are desirable for protection against cardiovascular disease, inflammation and mental illnesses (see, e.g., Ross et al. (2007) Lipids Health Dis. 6:21; Lands (1992) FASEB J. 6(8):2530; and U.S. Pat. No. 6,870,077). Evidence suggests that essential fatty acids, particularly EPA and DHA, in the form of food and nutritional supplements, play a role in preventing a number of disease states, including cardiovascular diseases, inflammation, mental health and behavioral diseases and disorders (see, e.g., Ross et al. (2007) Lipids Health Dis. 6:21; Lands (1992) FASEB J. 6(8):2530; U.S. Pat. No. 6,870,077; and Covington (2004) Am. Fam. Physician 70(1):133-140).

Conjugated fatty acids are PUFAs with two or more conjugated double bonds. Conjugated fatty acids can be used as nutritional supplements. Exemplary of conjugated fatty acids are conjugated linoleic acid (CLA), for example, 18:2 ω-7, 18:2 ω-6; conjugated linolenic acid, for example, 18:3 ω-6, 18:3 ω-5; and other conjugated fatty acids, for example, 18:3 ω-3, 18:4 ω-3, and 20:5 ω-6.

i. Omega-3 Fatty Acid Compounds

Exemplary of the PUFA-containing non-polar ingredients that can be used in the provided pre-gel concentrates are non-polar ingredients that contain one or more of the non-polar compound omega-3 (ω-3; n-3) fatty acids, for example, ingredients containing DHA and/or EPA fatty acids, for example, marine oils, e.g., fish oil, krill oil and algae oil; and ingredients containing ALA fatty acids, for example, flax seed oil.

Typically, oils and compositions containing long-chain PUFAs, such as DHA and EPA, are susceptible to oxidation, making them unstable and giving them an unpleasant taste. The ingredients and relative concentrations thereof, as well as the methods for making the pre-gel concentrates, contribute to desirable properties of DHA/EPA-containing compositions. For example, the ingredients and methods used to make the pre-gel concentrates and soft gel compositions provided herein minimize the “fishy” odor and/or taste of DHA/EPA-containing compositions and increase their stability over time. For example, the PUFA-containing fatty acid ingredients in the pre-gel concentrates can have low oxidation, contributing to these desirable properties.

(1) DHA/EPA

Exemplary of non-polar ingredients that contain one or more omega-3 fatty acids which can be used in the provided pre-gel concentrates are ingredients containing DHA and/or EPA, for example, marine oil, e.g., fish oil, krill oil and algae oil. Any oil containing DHA and/or EPA can be used. Exemplary non-polar ingredients that can be used in the pre-gel concentrates provided herein include non-polar ingredients that contain only DHA, for example, non-polar ingredients that contain between 10% or about 10% and 40% or about 40% DHA, between 25% or about 25% and 45% or about 45% DHA, or between 60% or about 60% and 90% or about 90% DHA, for example, at least 65% or about 65%, at least 70% or about 70%, at least 75% or about 75%, at least 80% or about 80%, at least 85% or about 85%, or at least 90% or about 90%, by weight (wt %), DHA. Exemplary non-polar ingredients that can be used in the pre-gel concentrates provided herein include non-polar ingredients that contain only EPA, for example, non-polar ingredients that contain between 5% or about 5% and 15% or about 15% EPA, or non-polar ingredients that contain not more than 10% or about 10% EPA. Exemplary non-polar ingredients that contain a mixture of DHA and EPA are suitable for use in the pre-gel concentrates provided herein, for example, compositions containing at least 20% or about 20% DHA and not more than 13% or about 13% EPA, by weight, of the non-polar ingredient; at least 35% or about 35% DHA and not more than 13% or about 13% EPA; at least 70% or about 70% DHA and not more than 13% or about 13% EPA; or the total amount of DHA and EPA represents at least 30% or about 30% of the non-polar ingredient, or at least 61% or about 61% of the non-polar ingredient.

(a) Fish Oils

Exemplary of the PUFA-containing non-polar ingredients that can be used in the provided pre-gel concentrates are oils derived from fish which contain DHA, EPA or both DHA and EPA. Particularly, cold water marine fish are a known source of omega-3 fatty acids (U.S. Pat. No. 4,670,285). Any fish oil containing DHA and/or EPA can be used as the non-polar ingredient in the provided pre-gel concentrates. Suitable fish oils containing DHA, EPA, or both DHA and EPA can be obtained from any of a number of commercial sources, for example, the fish oils available from Jedwards International, Inc., O3C Nutraceuticals, Ocean Nutrition Canada, Lipid Nutrition B.V., and GC Rieber Oils, any of which can be used with the provided pre-gel concentrates provided herein.

Fish oils typically are extracted from fish tissue, for example, frozen fish tissue. For example, the fish oil can be a tasteless fish oil, for example, a cod liver oil, which has been isolated from fish, for example, from cod liver, and then refined and deodorized, or in some other way treated so its taste becomes neutral, such as described in International Publication Nos. WO 00/23545 and WO 2004/098311. In one example, these fish oils are isolated from frozen fish tissue by a process that minimizes oxidation. Exemplary of such a tasteless fish oil is a fish oil sold under the trademark Denomega™ 100 (Borregaard Ingredients, Sarpsborg, Norway; distributed by Denomega Nutritional Oils AS, Boulder, Colo.). Typically, the tasteless fish oil, for example, cod liver oil, contains between or about between 25% and 35% omega-3 fatty acids, for example, 34% omega-3 fatty acids. In one example, the fish oil, for example, the Denomega™ 100 oil, contains 13% or about 13% DHA and 13% or about 13% EPA.

Also exemplary of the fish oils that can be included in the provided pre-gel concentrates are fish oils containing high amounts of omega-3 fatty acids, for example, high amounts of DHA. One example of such a fish oil contains at least or about at least 85% DHA, typically greater than 85% DHA, and at least or about at least 90% omega-3 fatty acids, typically greater than 90% omega-3 fatty acids. In another example, the fish oil can contain 98% PUFA with 89% omega-3 fatty acids, such as about 70% DHA and about 10% EPA, 8.9% omega-6 fatty acids, and 0.7% omega-9 fatty acids.

Exemplary of a fish oil containing high amounts of omega-3 fatty acids that can be used as the non-polar ingredient in the provided pre-gel concentrates is an omega-3 fish oil EE (O3C Nutraceuticals; supplied by Jedwards International Inc., Quincy, Mass.), which contains 98% polyunsaturated fatty acids (PUFAs), including 89% omega-3 fatty acids, such as 74.5% docosahexanoic (DHA) fatty acids and 9.3% eicosapentaenoic (EPA) fatty acids, 8.9% omega-6 fatty acids, and 0.7% omega-9 fatty acids, 0.1% saturated fatty acids, and 1.0% monounsaturated fatty acids. This fish oil contains 0.1% palmitic acid (16:0), 0.1% palmitoleic acid (16:1 ω-7), 0.1% stearic acid (18:0), 0.6% oleic acid (18:1 ω-9), 0.1% oleic acid (18:1 ω-7), 0.3% linoleic acid (18:2 ω-6), 0.2% linolenic acid (18:3 ω-3), 0.2% octadecatetraenoic acid (18:4 ω-3), 0.1% eicosanoic acid (20:1 ω-9), 0.1% eicosadienoic acid (20:2 ω-6), 0.2% eicosatrienoic acid (20:3 ω-6), 2.4% arachidonic acid (20:4 ω-6), 0.6% arachidonic acid (20:4 ω-3), 0.1% erucic acid (22:1 ω-11), 0.6% uncosapentaenoic acid (21:5 ω-3), 0.5% docosatetraenoic acid (22:4 ω-6), 5.4% docosapentaenoic acid (22:5 ω-6), 3.6% docosapentaenoic acid (22:5 ω-3), and 0.9% other fatty acids.

Also exemplary of a fish oil containing high amounts of omega-3 fatty acids that can be used in the provided pre-gel concentrates is Omega Concentrate 85 DHA TG Ultra (O3C Nutraceuticals AS, Oslo, Norway), which contains greater than 85% DHA (22:6 ω-3) and greater than 90% total omega-3 fatty acids, and is isolated from fatty fish species in the Eugraulidae, Clupeidae and Scombridae families. This fish oil is produced by purifying and concentrating the oils from these fish with gentle technologies to increase the concentration of omega-3 fatty acid DHA.

Any fish oil containing DHA and/or EPA can be used as the non-polar ingredient in the provided pre-gel concentrates. Exemplary of the fish oils that can be included is Eterna™ Omegasource™ Oil (supplied by Hormel Foods Specialty Products Division, Austin, Minn.), which contains at least 30% omega-3 fatty acids (DHA, EPA and ALA), is odorless, virtually free of cholesterol and bland in flavor. This fish oil contains about 28% DHA and EPA, typically 17% EPA and 11% DHA, and additionally contains 4.5% omega-6 fatty acids. Also exemplary of the fish oils that can be included in the provided pre-gel concentrates are Omega 30 TG Food Grade (Non-GMO) MEG-3™ Fish Oil (supplied by Ocean Nutrition Canada, Dartmouth, Nova Scotia, Canada), a kosher fish oil which contains about 30% DHA/EPA and Marinol C-38 (supplied by Lipid Nutrition B.V., Channahon, Ill.), which contains about 52% omega-3 fatty acids, including at least 38% DHA/EPA, more specifically includes about 22% EPA and 14% DHA. Also exemplary of fish oils are Marinol D-40 (supplied by Lipid Nutrition B.V., Channahon, Ill.), which contains about 40% DHA and 7% EPA; omega-3 fish oil 70TG that is 61% by weight DHA/EPA; fish oils sold by GC Rieber Oils (Kristiansund, Norway) that contain 30% or 65% DHA; ONC TG fish oil sold by Ocean Nutrition Canada (Dartmouth, Nova Scotia); Omevital™ 30% MP Gold, a fish oil that contains 30% DHA/EPA (Cognis, Monheim am Rhein, North Rhine-Westphalia, Germany); and a fish oil containing 60% DHA (sold by FINA LLC, Cincinnati, Ohio). Also exemplary of the fish oils are krill oils, such as those made according to International Publication No. WO 2007/080515.

(b) Algae Oil

Also exemplary of non-polar ingredients containing omega-3 PUFAs, particularly DHA (and optionally EPA), that can be used as the non-polar ingredient in the provided pre-gel concentrates are oils derived from microorganisms, for example, oils derived from marine dinoflagellates, such as microalgae, e.g., Crypthecodinium sp, particularly Crypthecodinium cohnii. Microalgae oils, like fish oils, are an excellent source of omega-3 fatty acids, particularly DHA (U.S. Pat. Nos. 5,397,591; 5,407,957; 5,492,938; and 5,711,983). Exemplary of oils derived from microalgae are the oils disclosed in (and oils made according to the methods described in) U.S. Pat. Nos. 5,397,591; 5,407,957; 5,492,938; and 5,711,983 and U.S. Publication No. 2007/0166411, including DHASCO® and DHASCO-S® (Martek Biosciences Corporation).

For example, U.S. Pat. No. 5,397,591 describes, inter alia, single-cell edible algae oils (and methods for making the oils) which contain at least 70% triglycerides, preferably containing more than 70% triglycerides, about 30-40% DHA, and lack EPA. These oils, isolated from Crypthecodinium cohnii, have 15-20% myristic acid, 20-25% palmitic acid, 10-15% oleic acid, 30-40% DHA, and 0-10% other triglycerides. U.S. Pat. No. 5,407,957 describes, inter alia, algae oils (and methods for making the oils) derived from Crypthecodinium cohnii, preferably containing greater than about 90% triglycerides, at least 35% DHA by weight (w/w), in one example, having 15-20% myristic acid, 20-25% palmitic acid, 10-15% oleic acid, 40-45% DHA, and 0-5% other oils. U.S. Pat. No. 5,492,938 describes, inter alia, single cell edible oils (and methods for making the oils) containing at least 70% triglycerides, which contain about 20-35% DHA and lack EPA, isolated from Crypthecodinium cohnii, in one example containing more than 70% triglycerides, having 15-20% myristic acid, 20-25% palmitic acid, 10-15% oleic acid, 30-40% DHA, and 0-10% other triglycerides. U.S. Pat. No. 5,711,983 describes, inter alia, single cell edible oils (and methods for making the oils) containing at least 70% triglycerides, which contain about 20-35% DHA and lack EPA, isolated from Crypthecodinium cohnii, in one example containing more than 70% triglycerides, having 15-20% myristic acid, 20-25% palmitic acid, 10-15% oleic acid, 30-40% DHA, and 0-10% other triglycerides.

Also exemplary of suitable algae oils are those microalgae oils disclosed, for example, in U.S. Pat. No. 6,977,166 and U.S. Publication No. US 2004/0072330. Any oil derived from dinoflagellate, for example, microalgae, which contains DHA, and optionally EPA, is suitable as an algae oil for use with the provided compositions, for example, V-Pure algae oil (Water4Life, Switzerland), which contains EPA and DHA, and Martek DHA™-S (supplied by Martek Biosciences Corporation, Columbia, Md.), derived from the marine alga Schizochytrium sp., containing not less than 35% DHA and 16.1% docosapentaenoic acid (22:5 ω-6), 1.3% eicosapentaenoic acid (20:5 ω-3), 0.6% arachidonic acid (20:4 ω-6), 1.6% linoleic acid (18:2 ω-6), 16.9% oleic acid (18:1 ω-9), and 19.8% other fatty acids.

(2) Flax Seed Oil—Omega 3 (ALA)

Also exemplary of the omega-3-containing non-polar ingredients used in the provided pre-gel concentrates is flaxseed oil (linseed oil). Flaxseed oils, which are good sources of omega-3 fatty acids, particularly alpha-linolenic acid, have been used as nutritional supplements. Flaxseed oils are produced by pressing the flax seed and refining the oil from the flax seeds. Exemplary of a flaxseed oil that can be used as the non-polar ingredient in the provided compositions is flaxseed oil derived from Linum usitatissimum L. Exemplary of flaxseed oils suitable for use in the pre-gel concentrates provided herein include flaxseed oil supplied by Sanmark LLC (Greensboro, N.C.; Sanmark Limited, Dalian, Liaoning Province, China), which contains not less than (NLT) 50% C18:3 alpha-linolenic acid, and further contains other fatty acids, for example, 3-8% C16:0 palmitic acid, 2-8% C18:0 stearic acid, 11-24% C18:1 oleic acid, 11-24% C18:2 linoleic acid and 0-3% other fatty acids. Also exemplary of a suitable flaxseed oil is a flaxseed oil containing 6% palmitic acid, 2.5% stearic acid, 0.5% arachidic acid, 19% oleic acid, 24.1% linoleic acid, 47.4% linolenic acid, and 0.5% other fatty acids.

The fatty acid composition of flaxseed oil can vary. Any flaxseed oil can be used as the non-polar ingredient in the provided pre-gel concentrates. For example, the flaxseed oil can contain at least or about at least 50%, at least or about at least 65%, or at least or about at least 70% alpha-linolenic acid. Exemplary of a flaxseed oil containing greater than 65% linolenic acid (of the total fatty acid content), for example, 70-80% or 70-75%, is the flaxseed oil described in U.S. Pat. No. 6,870,077.

ii. Omega-6 Fatty Acid Compounds

Also exemplary of the non-polar ingredients used in the provided pre-gel concentrates are ingredients containing omega-6 PUFAs, for example, gamma-linolenic acid (GLA), for example, borage oil and evening primrose (Oenothera biennis) oil, blackcurrant seed oil, hemp seed oil, fungal oil and spirulina extract. Any oil containing omega-6 fatty acids can be used in the provided pre-gel concentrates.

Exemplary of the omega-6-containing non-polar ingredients are ingredients containing GLA, for example, borage oil. GLA is an omega-6 PUFA, which primarily is derived from vegetable oils, for example, evening primrose (Oenothera biennis) oil, blackcurrant seed oil, hemp seed oil, and spirulina extract. GLA has been used as a nutritional supplement. It has been proposed that GLA has a role in treating various chronic diseases and in particular that it has anti-inflammatory effects (Fan and Chapkin (1998) J. Nutr. 128(9):1411-1414). In one example, the non-polar ingredient contains at least or about at least 22 wt % of GLA, for example, 22, 23, 24, 25, 26, 27, 28, 29, 30, 31, 32, 33, 34, 35, 36, 37, 38, 39, 40, 50, 60 wt % or more, by weight of GLA.

Borage (Borago officinalis), also known as “starflower,” is an herb with seeds containing high amounts of GLA. Exemplary of borage oils that can be used as a non-polar ingredient in the provided pre-gel concentrates are borage oils supplied by Sanmark LLC (Greensboro, N.C.; Sanmark Limited, Dalian, Liaoning Province, China), derived by pressing and isolating oil from the seeds of Borago officinalis L. This oil contains not less than (NLT) 22% C18:3 gamma-linolenic acid (GLA), 9-12% C16:0 palmitic acid, 3-5% C18:0 stearic acid, 15-20% C18:1 oleic acid, 35-42% C18:2 linoleic acid, 3-5% C20:1 ocosenoic acid, 1-4% C22:1 docosenoic acid, and 0-4% other fatty acids. Other borage oils can be used. Other GLA-containing oils also can be used as the non-polar ingredient in the pre-gel concentrates provided herein.

iii. Saw Palmetto Extract

Also exemplary of the non-polar ingredients used in the provided pre-gel concentrates is saw palmetto extract, a lipophilic extract of the ripe berries of the American dwarf palm (also called Serenoa repens or Sabal serrulata), which has been used to treat genitourinary and other diseases and to enhance sperm production, breast size and libido, as a mild diuretic, a nerve sedative, an expectorant and a digestive tract tonic, and particularly to treat benign prostate hyperplasia (BHP) (Ernst (2002) Acad. Clin. 136:42-53 and Gordon and Shaughnessy (2003) Comp. Alt. Med. 76(6):1281-1283). Saw palmetto extract is commercially available from a number of sources. Any saw palmetto lipid extract can be used in the provided pre-gel concentrates. Exemplary of the saw palmetto extract that can be used in the provided pre-gel concentrates is Saw Palmetto, Lipophilic Extract, commercially available from Natural Medicinals, Inc. (Felda, Fla.). This saw palmetto lipophilic extract is carbon dioxide extracted and, in one example, contains 85.9% total fatty acids, including 0.8% caproic acid, 2% caprylic acid, 2.4% capric acid, 27.% lauric acid, 10.3% myristic acid, 8.1% palmitic acid, 0.2% palmitoleic acid, 2% stearic acid, 26.7% oleic acid, 4.9% linoleic acid, 0.7% linolenic acid, 0.42% phytosterols, including 0.42% beta sitosterol, 0.09% campesterol, 0.03% stigmasterol, and 0.2% moisture. Other sources of saw palmetto extract can be used in the pre-gel concentrates provided herein.

iv. Conjugated Linoleic Acid (CLA)

Also exemplary of the PUFA-containing non-polar ingredients that can be used in the provided pre-gel concentrates are non-polar ingredients containing conjugated fatty acids. Conjugated fatty acids are PUFAs with two or more conjugated double bonds. Conjugated fatty acids can be used as nutritional supplements. Exemplary non-polar ingredients containing conjugated fatty acids are ingredients containing conjugated linoleic acid (CLA), for example, 18:2 ω-7 and 18:2 ω-6; conjugated linolenic acid, for example, 18:3 ω-6 and 18:3 ω-5; and other conjugated fatty acids, for example, 18:3 ω-3, 18:4 ω-3 and 20:5 ω-6. CLA refers to a family of linoleic acid isomers found primarily in meat and dairy products of ruminants. Typically, the CLA ingredients contain a mixture of different CLA isomers, for example, C18:2 CLA c9, t11, CLA t10, c12 and other CLA isomers. Exemplary of a CLA that can be used as a non-polar ingredient in the provided pre-gel concentrates is CLA (70%) commercially available from Sanmark, LTD (Dalian, Liaoning Province, China; product code 01057-A80). This CLA is a clear white to pale yellow oil and has the following fatty acid composition: NMT (not more than) 9.0% C16:0 palmitic acid, NMT 4.0% stearic acid, NMT 15.0% C18:1 oleic acid, NMT 3.0% C18:2 linoleic acid, NLT (not less than) 80% C18:2 CLA (including the following isomers: NLT 37.5% C18:2 CLA c9, t11; 37.5% C18:2 CLA t10, c12; and NMT 5.0% other CLA isomers); and NMT 5.0% other fatty acids. Another exemplary CLA compound is a CLA that contains 74.5% CLA (Clarinol® CLA, Stepan Lipid Nutrition, Maywood, N.J.). Other CLA-containing ingredients can be used in the pre-gel concentrates provided herein.

b) Non-Essential Fatty Acids

Exemplary of fatty acid-containing non-polar ingredients that can be included the provided pre-gel concentrates are non-polar ingredients containing non-essential fatty acids and non-essential fatty acid derivatives. These fatty acids and derivatives can be monounsaturated or polyunsaturated. Non-essential fatty acids include, but are not limited to, fatty acids such as omega-5 fatty acids, omega-7 fatty acids, omega-9 fatty acids, and fatty acid derivatives, such as derivatives of omega-5 fatty acids, omega-7 fatty acids, and omega-9 fatty acids. The non-essential fatty acids that can be used in the provided pre-gel concentrates can be natural or synthetic non-essential fatty acids and derivatives thereof.

Exemplary of non-polar ingredients containing omega-5 fatty acids are ingredients containing punicic acid (18:3 ω-5) and 9-tetradecenoic acid (myristoleic acid), and/or derivatives of omega-5 fatty acids, for example, esters of omega-5 fatty acids, such as ingredients containing cetyl esters of omega-5 fatty acids, e.g., cetyl myristoleate. Cetyl myristoleate (CMO) is the ester produced by the reaction of cis-9-tetradecenoic acid (myristoleic acid) with 1-hexadecanol (cetyl alcohol). An exemplary compound containing non-essential fatty acid derivatives is Myristin®, a compound that contains 40% CMO and 60% cetyl oleate and other cetyl esters, sold by EHP Products, Inc., Mt. Pleasant, S.C. Exemplary of ingredients containing omega-7 fatty acids are ingredients containing vaccenic acid (18:1 trans-11), palmitoleic acid (9-hexadecenoic acid) and derivatives thereof. Exemplary of ingredients containing omega-9 fatty acids are ingredients containing omega-9 fatty acids, such as the monounsaturated oleic acid (18:1 ω-9), eicosatrienoic acid (20:3 ω-3), mead acid (20:3 ω-9), erucic acid (22:1 ω-9), nervonic acid (24:1 ω-9) and derivatives thereof. Exemplary of a non-polar ingredients containing omega-9 fatty acids that can be used in the pre-gel concentrates provided herein is the oleic acid compound sold by KIC Chemicals, Inc. (New Paltz, N.Y.), which contains 70% oleic acid. Any non-polar ingredients that contains any non-essential fatty acid can be used in the provided pre-gel concentrates.

c) Phospholipids

Exemplary of the fatty acid-containing non-polar ingredients that can be included in the provided pre-gel concentrates are phospholipids. Phospholipids are amphipathic lipid-like molecules, generally with a hydrophobic portion at one end of the molecule and a hydrophilic portion at the other end of the molecule, that typically contain a central glycerol moiety with two fatty acid chains and a phosphate group that can be further derivatized.

A number of phospholipids can be included ingredients in the provided pre-gel concentrates. For example, the phospholipid can be a phospholipid derivative containing lecithins of natural origin, for example, soy or egg lecithins, phospholipids of natural origin, for example, soy or egg phospholipids, synthetic phospholipids, or mixtures thereof. Exemplary phospholipids include phosphatidylcholine (PC), phosphatidylethanolamine (PE), distearoylphosphatidylcholine (DSPC), phosphatidylserine (PS), phosphatidylglycerol (PG), phosphatidic acid (PA), phosphatidylinositol (PI), sphingomyelin (SPM), or a combination thereof. Typically, the phospholipid is phosphatidylcholine (PC), which sometimes is referred to by the general name “lecithin.” Exemplary of the phospholipids that can be included in the provided pre-gel concentrates are the phospholipids sold by Lipoid, LLC (Newark, N.J.), for example, purified egg lecithins, purified soybean lecithins, hydrogenated egg and soybean lecithins, egg phospholipids, soybean phospholipids, hydrogenated egg and soybean phospholipids, synthetic phospholipids, PEGylated phospholipids and phospholipid blends. Exemplary of phosphatidylcholine compounds that can be included in the provided pre-gel concentrates are the phosphatidylcholine composition sold by Lipoid, LLC (Newark, N.J.), under the name Lipoid S100, which is derived from soy extract and contains greater than or greater than about 95% phosphatidylcholine; the phosphatidylcholine sold under the name Lecithin Ultralec® P (ADM Natural Health and Nutrition, Decatur, Ill.); and Phospholipon®, a lecithin fraction with approximately 95% phosphatidylcholine, sold by Lipoid, Steinhausen, Switzerland, and American Lecithin Company, Oxford, Conn.

Also exemplary of phospholipids that can be included in the pre-gel concentrates provided herein are marine phospholipids, which are phospholipids derived from marine organisms. Marine phospholipids commonly contain PUFAs with two or more unsaturated bonds, in particular with four or more unsaturated bonds. The lipid moieties of phospholipids are commonly of the omega-3 type, which often exhibit enhanced stability, e.g., oxidative stability, when incorporated into phospholipids.
2) Coenzyme Q Compounds

Exemplary of the non-polar ingredients that can be included in the pre-gel concentrates provided herein are ingredients containing the non-polar compound coenzyme Q, for example, coenzyme Q10 (also called coQ10, ubiquinone, ubidecarenone, ubiquinol and vitamin Q10). Coenzyme Q ingredients are benzoquinone compounds containing isoprenoid units. The number of isoprenoid units in each of the different CoQ species is indicated with a number following CoQ. For example, coQ10 contains 10 isoprenoid units. Coenzyme Q10 is a predominant coenzyme Q species. CoQ10 has electron-transfer ability and is present in cellular membranes, such as those of the endoplasmic reticulum, peroxisomes, lysosomes, vesicles and the mitochondria. A decrease in natural coQ10 synthesis has been observed in sick and elderly people. Because of this observation and its potent antioxidant properties, coQ10 is used as a dietary supplement and a treatment for diseases such as cancer and heart disease. CoQ10, however, exhibits relatively poor bioavailability.

Coenzyme Q can exist in two different forms: an oxidized form and a reduced form. When the oxidized form of a coenzyme Q species is reduced by one equivalent, i.e., partially reduced, it becomes a ubisemiquinone (semiquinone), denoted QH, which contains a free radical on one of the oxygens in the benzene ring of the benzoquinone. Further oxidation of QH results in ubiquinol, the fully reduced, active form of coQ10. Both oxidized and reduced coenzyme Q-containing ingredients can be used as non-polar ingredients in the provided pre-gel concentrates. CoQ10 typically refers to the oxidized form of coQ10, which also is referred to as ubidecarenone, as opposed to the partially reduced form of coQ10, referred to as ubisemiquinone, and the fully reduced form of coQ10, referred to as ubiquinol. Both the reduced (i.e., coQ10, ubiquinone, ubidecarenone) and oxidized forms (i.e., ubisemiquinone and ubiquinol) of coQ10 are exemplary of the coenzyme Q species that can be used as non-polar ingredients in the provided pre-gel concentrates.

CoQ10-containing ingredients are available commercially. Any coQ10 compound or oxidized coQ10 compound can be used with the provided pre-gel concentrates. Exemplary of the coQ10 ingredients that can be used are coenzyme Q10 compounds containing greater than 98% or greater than about 98% ubidecarenone, for example, the compound sold under the name Kaneka Q10™ (USP Ubidecarenone) by Kaneka Nutrients, L.P. (Pasadena, Tex.). The compound sold under the name Kaneka Q10™ is fermented entirely from yeast and is identical to the body's own coQ10 and free from the cis isomer found in some synthetically produced coQ10 compounds. Another exemplary ingredient includes non-polar ingredients containing the reduced form of coQ10, ubiquinol, for example, the compound Kaneka Ubiquinol® sold by Kaneka Nutrients (Pasadena, Tex.). Any coQ10 ingredients containing the reduced or oxidized form of coQ10 can be used in the provided pre-gel concentrates provided herein.

3) Phytochemical-Containing Compounds

Exemplary of the non-polar ingredients included in the provided pre-gel concentrates are phytochemical-containing ingredients, for example, phytosterols (plant sterols), phytoestrogens, for example, genistein and daidzein, flavonoids, for example, isoflavones, for example, soy isoflavones, phytoalexins, for example, resveratrol (trans-3,5,4′-trihydroxystilbene), and red clover extract.

a) Phytosterols

Exemplary of the phytochemical-containing compounds that can be used in the provided pre-gel concentrates are phytosterols (plant sterols). Plant sterols are structurally similar to cholesterol and have been found to reduce the absorption of dietary cholesterol, which can affect the levels of serum cholesterol. According to the U.S. Food and Drug Administration (FDA), two servings per day, each containing 0.4 grams of plant sterols, for a total daily intake of at least 0.8 grams, as part of a diet low in saturated fat and cholesterol, is reported to reduce the risk of heart disease. Thus, plant sterols are used in nutritional supplements.

Any phytosterol-containing compound can be used as a non-polar ingredient in the provided pre-gel concentrates. Exemplary of the phytosterol-containing ingredients that can be used in the provided pre-gel concentrates are ingredients containing plant sterols, for example, the ingredient sold under the name CardioAid™, distributed by B&D Nutrition and manufactured by ADM Natural Health and Nutrition, Decatur, Ill. This ingredient contains kosher, pareve, and halal plant sterols that are produced under current food GMPs and contain a minimum of 95% plant sterols, which can include up to 5 plant sterols. The ingredient can contain, for example, 40-58% beta sitosterol, 20-30% campesterol, 14-22% stigmasterol, 0-6% brassicasterol, and 0 5% sitostanol. The ingredient further can contain tocopherols, for example, 0-15 mg/g tocopherols. The ingredient is tested and is negative for microorganisms, such as Salmonella, E. coli and Staphylococcus aureus.

b) Flavonoids

Exemplary of the phytochemical-containing compounds used as non-polar ingredients in the provided concentrates are flavonoids. Flavonoids are a class of plant secondary metabolites that have a general structure of a 15-carbon skeleton, which consists of two phenyl rings and a heterocyclic ring, that can be abbreviated C6-C3-C6. Exemplary flavonoid compounds include bioflavonoids, isoflavonoids, and neoflavonoids.

Exemplary of a flavonoid is resveratrol, or trans-resveratrol (trans-3,5,4′-trihydroxystilbene), a phytoalexin that is naturally produced by several plants, such as the Japanese knotweed, and also is found in the skin and seeds of grapes, numerous berries, including mulberries, blueberries, bilberries and cranberries, and in peanuts. This polyphenolic compound can act as an antioxidant and additionally, can aid in cancer prevention and reduction of cardiovascular disease.

Any resveratrol-containing ingredient can be included as a non-polar ingredient in the provided compositions. Exemplary of resveratrol-containing ingredients that can be used as non-polar ingredients in the provided compositions are ingredients containing trans-resveratrol, for example the compound sold under the name ReserveNature™, sold by Jiaherb, Shaanxi, China. This compound contains trans-resveratrol from the botanical source Polygonum cuspidatum (Japanese knotweed). This resveratrol ingredient contains a minimum of 98.5% trans resveratrol and does not contain emodin. The ingredient is tested and is negative for microorganisms, such as Salmonella, E. coli, yeast and mold. Another exemplary resveratrol ingredient is the resveratrol sold by Maxsun Industries (Walnut, Calif.).

An exemplary flavonoid that can be included in the pre-emulsion concentrates provided herein is quercetin. Quercetin is a plant pigment that is found in fruits, vegetables, leaves, and grains. Quercetin can act as an antiviral agent, reduce asthma symptoms, minimize eczema, and may have anti-inflammatory properties. An exemplary quercetin ingredient is the quercetin sold by Pure Assay Ingredients (Walnut, Calif.).

4) Carotenoid-Containing Compounds

Exemplary of the non-polar ingredients used in the provided pre-gel concentrates are carotenoid-containing ingredients, for example, carotenoids, including hydrocarbons (carotenes) and oxygenated, alcoholic derivatives of hydrocarbons (xanthophylls), for example, beta-carotene, mixed carotenoids complex, lutein, zeaxanthin, cryptoxanthin, for example, beta-crytoxanthin, lycopene, astaxanthin, bixin, canthaxanthin, capsanthin, capsorubin, apo-carotenal, beta-12′-apo-carotenal, “carotene” (mixture of alpha- and beta-carotene), gamma-carotene, ciolerythrin and esters of hydroxyl- or carboxyl-containing members thereof Carotenoids are efficient free-radical scavengers, or anti-oxidants, and are capable of enhancing the vertebrate immune system.

a) Carotenes

Exemplary of the carotenoid-containing compounds used as non-polar ingredients in the provided pre-gel concentrates are carotenes, for example, alpha-carotene, beta-carotene, lycopene, and mixtures thereof. Any carotene-containing compound can be used as a non-polar ingredient in the provided compositions. Exemplary of a carotene-containing compound that can be used in the provided pre-gel concentrates is lycopene, sold by Zhejiang Medicine CO., LTD (Xinchang Pharmaceutical Factory, Xinchang, China), a purple or red crystalline powder containing not less than 70% all E-lycopene, not more than 23% 5-Z-lycopene and not more than 9% related substances.

b) Xanthophylls

Exemplary of the carotenoid-containing compounds used as non-polar ingredients in the provided pre-gel concentrates are xanthophylls, for example, astaxanthin, neoxanthin, violaxanthin, α- and β-cryptoxanthins, lutein and zeaxanthin. Xanthophylls, or phylloxanthins, are oxygen-containing carotenoids that are typically yellow pigments. Any xanthophyll can be used as a non-polar ingredient in the provided pre-gel concentrates. An exemplary xanthophyll included in the pre-gel concentrates provided herein is astaxanthin, for example, the astaxanthins AstraREAL® (sold by Fuji Health Science, Burlington, N.J.), AstaPure® (sold by Alga Technologies, Hevel Eilot, Israel), and BioAstin® (sold by Cyanotech, Kailua-Kona, Hi.). Unlike other carotenoids, astaxanthin is not converted to vitamin A (retinol) in the human body, but has potent antioxidant activity and can be beneficial in cardiovascular, immune, inflammatory and neurodegenerative diseases. Other exemplary xanthophyll ingredients that can be included in the provided pre-gel concentrates are lutein and zeaxanthin, sold under the name Xanmax®-80 (Lutein crystals), by Katra Phytochem (India) Private Limited, Bangalore, India, containing 80% lutein and 4.5% zeaxanthin.

5) Micronutrient-Containing Compounds

Exemplary of the non-polar ingredients included in the provided pre-gel concentrates are micronutrient-containing ingredients, for example, vitamins, including vitamins A, B, C, D, E and K, and corresponding provitamins and vitamin derivatives with an action resembling that of vitamin A, B, C, D, E or K, yerba mate, ginseng and ginkgo biloba.

a) Vitamins

Exemplary of the vitamins included in the provided pre-gel concentrates are fat-soluble vitamins, for example, vitamins A, B, C, D, E and K, and corresponding provitamins and vitamin derivatives, such as esters, with an action resembling that of vitamin A, B, C, D, E or K. Exemplary vitamins include retinol (vitamin A) and pharmaceutically acceptable derivatives thereof, for example, palmitate ester of retinol and other esters of retinol, for example, vitamin A palmitate; B vitamins, for example, thiamin (vitamin B1), riboflavin (vitamin B2), niacin (vitamin B3), pantothenic acid (vitamin B5), pyridoxine (vitamin B6), biotin (vitamin B7), folic acid or folate (vitamin B9), and cyanocobalamin, cobalamin, or reduced forms of cobalamin (vitamin B12); calciferol (vitamin D) and its pharmaceutically acceptable derivatives thereof, for example, for example, cholecalciferol (vitamin D3), and precursors of vitamin D; d-alpha tocopherol (vitamin E) and derivatives thereof, including pharmaceutical derivatives thereof, for example, tocotrienols, d-alpha tocopherol acetate and other esters of d-alpha tocopherol; K vitamins, for examples, phylloquinone or phytonadione (vitamin K1) and menaquinone (vitamin K2), including the MK-4, MK-7, MK-8, and MK-9 forms; and ascorbyl palmitate, a fat-soluble version of vitamin C.

Any vitamin can be used as a non-polar ingredient in the provided pre-gel concentrates. Exemplary of the vitamins that can be used in the provided pre-gel concentrates are vitamin A palmitate, for example, vitamin A palmitate containing 1.7 mIU/g, produced by DSM Nutritional Products, Inc., Belvidere, N.J., and distributed through Stauber Performance Ingredients, Inc., Fullerton, Calif.; vitamin D3, for example, vitamin D3 in corn oil, containing about 1 mIU/g, produced by DSM Nutritional Products, Inc., Parsippany, N.J.; vitamin K2, for example, vitamin K2 (as MK-7), such as sold by NattoPharma®, Metuchen, N.J.; vitamin E (d-alpha-tocopherol), for example vitamin E oil containing 1000 IU/g vitamin E, sold as Novatol™ 5-67 by ADM Natural health and Nutrition, Decatur, Ill.; vitamin B12; vitamin B1; vitamin B3; vitamin B5; and vitamin B6. Vitamin non-polar ingredients are typically added to the pre-gel concentrates in amounts such that one serving of the soft gel composition provides an amount of the vitamin that corresponds to the dietary reference intakes.

6) Boswellia Extracts

Exemplary of non-polar ingredients included in the provided pre-gel concentrates are non-polar ingredients containing extracts of a Boswellia plant or a boswellic acid or derivative thereof. Extracts of the Boswellia family of plants, including, for example, Boswellia Serrata, exhibit anti-inflammatory, anti-arthritic and anti-ulcerogenic activity (see, e.g., U.S. Pat. No. 6,589,516). Extracts derived from Boswellia plants and suitable for use in the pre-gel concentrates provided herein include extracts derived from Boswellia Cartenii, Boswellia Frereana, Boswellia Bhau-dajaina, Boswellia Serrata, and Boswellia Thurifera. The extracts derived from Boswellia plants can be gums, oleo-gums, resins, essential oils and residues, or mixtures thereof. A typical extract of a Boswellia plant suitable for use herein includes at least one boswellic acid, for example, acetyl-11-keto-β-boswellic acid (AKBA). Exemplary of a Boswellia extract-containing compound that can be used as the non-polar ingredient in the provided pre-gel concentrates is ApresFLEX®, a compound that includes a Boswellia serrata extract that contains acetyl-11-keto-β-boswellic acid (AKBA), sold by PLT Health Solutions, Morristown, N.J.

7) Alkaloids

Exemplary of non-polar ingredients in the provided pre-gel concentrates are non-polar ingredients containing an alkaloid, for example, any edible or food-approved alkaloid. Exemplary alkaloids include, but are not limited to, caffeine, synephrine, vinpocetine, and γ-aminobutyric acid (GABA) derivatives, e.g., 4-amino-3-phenylbutyric acid (i.e., phenibut). Suitable alkaloids for inclusion in the provided pre-gel concentrates are a matter of design choice and well within the skill of the skilled artisan. The alkaloid-containing non-polar ingredients include caffeine that is added in the form of caffeine anhydrous, such as the Caffeine Anhydrous powder, a white crystalline powder sold by Pacific Rainbow International, Inc., City of Industry, Calif. Other exemplary non-polar ingredient containing alkaloids include herbal extracts, medicinal extracts and compounds from plants and drugs.

8) Cannabinoids

Cannabinoids and cannabinoid-containing ingredients are exemplary of non-polar ingredients that can be included the pre-gel concentrates provided herein. Cannabinoids include phytocannabinoids (found in the Cannabis sativa plant and some other plants), endocannabinoids (produced naturally in the body by humans and animals), and synthetic cannabinoids. Cannabinoids that can be included in the pre-gel concentrates provided herein can be natural cannabinoids, synthetic cannabinoids, semi-synthetic cannabinoids, or mixtures thereof. Actual or potential therapeutic applications for cannabinoids include the treatment of multiple sclerosis and other forms of muscular spasm, migraine headache, glaucoma, asthma, inflammation, insomnia, high blood pressure, nausea and vomiting, and the stimulation of appetite. Other potential therapeutic applications include the use of cannabinoids as oxytoxic, anxiolytic, anti-convulsive, anti-depressive, anti-psychotic, and anti-cancer agents.

Exemplary phytocannabinoids derived from the Cannabis sativa plant (commonly known as marijuana) are the terpenophenolic compounds Δ⁹-tetrahydrocannabinol (THC), Δ⁸-tetrahydrocannabinol (Δ⁸-THC) and other compounds structurally related to THC, cannabidiol (CBD), cannabigerol (CBG), cannabichromene (CBC), cannabinol (CBN), cannabicyclo (CBL), cannabivarin (CBV), tetrahydrocannabivarin (THCV), cannabidivarin (CBDV), cannabielsoin (CBE), cannabicitran (CBT), cannabinodiol (CBDL), cannabichromevarin (CBCV), cannabigerovarin (CBGV), cannabigerol monoethyl ether (CBGM), and mixtures and derivatives thereof, for example, nabiximols (Sativex®), a mixture of THC and CBD. Suitable phytocannabinoids also include those derived from plants other than Cannabis sativa, such as, for example, lipophilic alkamides (alkylamides) derived from Echinacea plants, and other cannabinoids derived from plants including, but not limited to, Echinacea purpurea, Echinacea angustifolia, Echinacea pallida, Acmela oleracea, Helichrysum umbraculigerum, and Radula marginate plants.

Endogenous cannabinoids are lipid-like substances produced in the brain and peripheral tissues that bind to and activate cannabinoid receptors present in the cell membrane, including, but not limited to, arachidonate acid-based lipids such as anandamide (N-arachidonoylethanolamide, AEA), 2-arachidonoylglycerol (2-AG), noladin ether (2-arachidonyl glyceryl ether), N-arachidonoyl dopamine (NADA), and virodhamine (OAE).

Synthetic cannabinoids are among the cannabinoids that can be included as non-polar ingredients. Synthetic cannabinoids include any compound having a cannabinoid-like structure or that produces effects similar to those of cannabinoids that is manufactured using chemical means, including, for example, synthetic Δ⁹-THC; dronabinol (Marinol®; (6aR-trans)-6a,7,8,10a-tetrahydro-6,6,9-trimethyl-3-pentyl-6H-dibenzo[b,d]pyran-1-ol); nabilone (Cesamet™; (±)-trans-3-(1,1-dimethylheptyl)-6,6a,7,8,10,10a-hexahydro-1-hydroxy-6-6-dimethyl-9H-dibenzo [b,d]pyran-9-one); dexanabinol ((6aS,10aS)-9-(hydroxymethyl)-6,6-dimethyl-3-(2-methyloctan-2-yl)-6a,7,10,10a-tetrahydrobenzo[c]chromen-1-ol); ajulemic acid (Resunab™; (6aR,10aR)-3-(1,1-dimethylheptyl)-6a,7,10,10a-tetrahydro-1-hydroxy-6,6-dimethyl-6H-dibenzo(b,d)pyran-9-carboxylic acid); cannabinor ((E)-4-(2-((1R,2R,5R)-6,6-dimethyl-4-oxobicyclo[3.1.1]heptan-2-yl)-3-hydroxy-5-(2-methyloctan-2-yl)phenoxy)-4-oxobut-2-enoic acid); HU 308 ([(1R,2R,5R)-2-[2,6-dimethoxy-4-(2-methyloctan-2-yl)phenyl]-7,7-dimethyl-4-bicyclo[3.1.1]hept-3-enyl]methanol); rimonabant (Acomplia™; 5-(4-chlorophenyl)-1-(2,4-dichloro-phenyl)-4-methyl-N-(piperidin-1-yl)-1H-pyrazole-3-carboxamide); taranabant (MK-0364; N-[(2S,3S)-4-(4-chlorophenyl)-3-(3-cyanophenyl)-2-butanyl]-2-methyl-2-{[5-(trifluoromethyl)-2-pyridinyl]oxy}propanamide); levonantradol ([(6S,6aR,9R,10aR)-9-hydroxy-6-methyl-3-[(2R)-5-phenylpentan-2-yl]oxy-5,6,6a,7,8,9,10,10a-octahydrophenanthridin-1-yl]acetate); WIN55212-2 ((R)-(+)-[2,3-dihydro-5-methyl-3-(4-morpholinylmethyl) pyrrolo[1,2,3-de]-1,4-benzoxazin-6-yl]-1-napthalenylmethanone); HU 331 (3-hydroxy-2-[(1R)-6-isopropenyl-3-methyl-cyclohex-2-en-1-yl]-5-pentyl-1,4-benzoquinone); and any other compound having a cannabinoid-based structure or that produces effects similar to those of cannabinoids that is manufactured using chemical means.

9) Hops-Containing Compounds

Exemplary of non-polar ingredients that can be included in the provided pre-gel concentrates are ingredients that contain hops (Humulus lupulus L.), including ingredients isolated or derived from hops, such as extracts of hops cones, for example, hops oils, hops resins or hops resin derivatives, hops acids or hops acid derivatives, or mixtures thereof. Hops oils include, but are not limited to, humulene, beta-caryophyllene, mycrene, farnescene, gamma-cadinene, alpha-selinene, and alpha-cadinene. Hops contain alpha-acids, such as humulone (α-lupulic acid), cohumulone, adhumulone, hulupone, and isoprehumulone, and beta-acids, such as lupulone, colupulone, adlupulone, tetrahydroisohumulone, and hexahydrocolupulone. Both alpha- and beta-acids have demonstrated antibacterial, antioxidant, and antiinflammatory properties. An exemplary non-polar ingredient containing hops is Perluxan™, a compound containing a supercritical extract of hops cones that includes a minimum of 30% alpha-acids (including humulone, cohumulone, adhumulone, iso-cohumulone and iso-adhumulone) and 10% beta-acids (including lupulone and colupulone), such as sold by Pharmachem Laboratories, Kearny, N.J.

10) Antioxidants

Exemplary of non-polar ingredients that can be included in the pre-gel concentrates provided herein are ingredients that contain an antioxidant or have antioxidant properties, for example, a molecule that is capable of inhibiting the oxidation of other molecules. Antioxidants include molecules that scavenge free radicals. Suitable antioxidants include those that are used as ingredients in dietary supplements. The antioxidant can be a natural antioxidant or a synthetic antioxidant.

Examples of antioxidants include, but are not limited to hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polyphenols, nonflavonoid phenolics, isothiocyanates, vitamins and vitamin cofactors, such as vitamin A, vitamin C, vitamin E, vitamin E phosphate and ubiquinone (ubidecarenone, coenzyme Q, coenzyme Q10), ubiquinol, pyrroloquinoline quinone (PQQ), ascorbic acid, citric acid, rosemary oil, minerals, such as mineral selenium and manganese, melatonin, α-carotene, β-carotene, lycopene, lutein, zeanthin, crypoxanthin, resveratrol, eugenol, quercetin, catechin, gossypol, hesperetin, curcumin, turmeric, turmeric/curcumin blend, ferulic acid, thymol, hydroxytyrosol, thyme, olive oil, lipoic acid, including alpha-lipoic acid, glutathione, oxalic acid, tocopherol, tocopherol-derived compounds, di-alpha-tocopheryl phosphate, tocotrienols, butylated hydroxyanisole, butylated hydroxytoluene, ethylenediaminetetraacetic acid, tert-butylhydroquinone, acetic acid, pectin, zeaxanthin, astaxanthin, canthaxanthin, saponins, limonoids, kaempferol, myricetin, isorhamnetin, proanthocyanidins, quercetin, rutin, luteolin, apigenin, tangeritin, naringenin, eriodictyol, flavan-3-ols (e.g., anthocyanadins), gallocatechins, epicatechin and its gallate forms, epigallocatechin and its gallate forms, theaflavin and its gallate forms, thearubigins, isoflavone phytoestrogens, genistein, daidzein, glycitein, anythocyanins, delphinidin, malvidin, pelargonidin, peonidin, and hops (Humulus lupulus L.)-containing ingredients. In one example, the antioxidant includes ubiquinol. In another example, the antioxidant includes alpha-lipoic acid. In another example, the antioxidant includes pyrroloquinoline quinone (PQQ). In yet another example, the antioxidant includes a turmeric/curcumin composition.

Any non-polar ingredient that is an antioxidant or has antioxidant properties can be included in the provided pre-gel concentrates. Exemplary of an antioxidant that can be used in the provided pre-gel concentrates is alpha-lipoic acid, for example, the alpha-lipoic acids sold by NutriChem Resources Company (Walnut, Calif.) and Zhejiang Medicines & Health Products Import & Export Co., Ltd (Hangzhou, China), and any other alpha-lipoic acid. Another exemplary antioxidant that can be used in the provided pre-gel concentrates is pyrroloquinoline quinone (PQQ), such as PureQQ, sold by Nascent Health Science (Allentown, N.J.). Exemplary of a non-polar ingredient that contains antioxidants that can be included in the provided pre-gel concentrates is a turmeric/curcumin composition, for example, the turmeric/curcumin composition that is 95% curcumin, sold by Siddharth International, Mumbai, India.

11) Additional Non-Polar Compounds

The pre-gel concentrates provided herein can contain additional non-polar ingredients other than and/or in addition to the non-polar compounds and ingredients listed above. Non-polar ingredients include small molecule drugs, such as compounds that can induce, promote or enhance one or more effects, such as upon delivery to a subject or upon administration to a subject, for example, sympathomimetic effects, stimulatory effects, vasoconstriction, decongestion (e.g., bronchial or nasal decongestion), increased energy, endurance, mood-enhancement, appetite suppression or stimulation and/or weight loss. These non-polar ingredients can include, but are not limited to, plant extracts, particularly those with medicinal and herbal effects. Typically, the additional non-polar ingredients are food-approved, i.e., edible or ingestible, non-polar ingredients, for example, non-polar ingredients that are safe and/or approved for human consumption.

b. Surfactants

In addition to the one or more non-polar ingredients, each of the provided pre-gel concentrates contains at least one surfactant. The pre-gel concentrates contain from about or at 5% to 50%, by weight, such as least 5%, 6%, 7%, 8%, 9%, 10%, 11%, 12%, 13%, 14%, 15%, 16%, 17%, 18%, 19%, 20%, 25%, 28%, 30%, 35%, 40%, such as 10% to 35%, 15% to 40%, or 15% to 30%, of a surfactant.

Surfactants are molecules that contain both hydrophobic and hydrophilic portions. In general, when placed in a lipophilic medium, surfactants aggregate to form micelles, which surround and contain the non-polar compounds. The hydrophilic portions of the surfactant molecules are oriented toward the outside of the micelle, in contact with the aqueous medium, while the hydrophobic portions of the surfactant molecules are oriented toward the center of the micelle, in contact with the non-polar compounds contained in the center of the micelle. The micelles can contain more than one surfactant.

Exemplary surfactants include those where the hydrophobic portion of the surfactant molecule is a hydrophobic tail and the hydrophilic portion is a hydrophilic head. Exemplary of suitable surfactants include, but are not limited to, vitamin E-derived surfactants, such as tocopherol and/or tocotrienol-derived surfactants, in which the vitamin E moiety represents the hydrophobic region of the surfactant, and is attached, via a linker, to another moiety, such as a polyalkylene glycol moiety, for example, a polyethylene glycol (PEG) moiety, that provides the hydrophilic portion of the surfactant. Vitamin-E derived surfactants include, but are not limited to, tocopherol derived surfactants, including polyalkylene glycol derivatives of tocopherol, typically polyethylene glycol (PEG) derivatives of tocopherol, such as tocopherol polyethylene glycol succinate (TPGS), TPGS analogs, TPGS homologs and TPGS derivatives. Alternatively, the surfactants can be other PEG derivatives having similar properties, for example, PEG derivatives of sterols, e.g., a cholesterol or a sitosterol (including, for example, any of the PEG derivatives disclosed in U.S. Pat. No. 6,632,443) or PEG derivatives of other fat-soluble vitamins, for example, some forms of vitamin A (e.g., retinol) or vitamin D (e.g., vitamins D1-D5).

1) Polyalkylene Glycol Derivatives of Vitamin E

Exemplary of the surfactants in the pre-gel concentrates described herein are water-soluble vitamin E derivatives, such as polyalkylene glycol derivatives of vitamin E, such as polyethylene glycol (PEG) derivatives of vitamin E, for example, PEG derivatives of tocopherols or tocotrienols. PEG derivatives of vitamin E can contain one or more tocopherol or tocotrienol, attached to one or more PEG moiety via a linker, for example, a dicarboxylic acid linker. Exemplary dicarboxylic acid linkers include succinic acid and succinic anhydride. An exemplary polyethylene glycol derivative of vitamin E is shown schematically below:

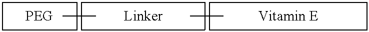

where the line between the PEG and the linker, and the line between the linker and the vitamin E moiety, each independently represent a covalent bond, for example, a covalent bond that forms an ester, ether, amide or thioester.

Typically, the vitamin E-PEG derivatives are made by covalently attaching the PEG moiety, such as by esterification, to a vitamin E-linker conjugate (e.g., a tocopherol-linker conjugate). The vitamin E-linker conjugate can be formed through esterification of the hydroxyl group of the vitamin E moiety with a carboxylic acid group of a linker, such as a dicarboxylic acid linker. In one example, the vitamin E-linker conjugate can be a tocopherol-linker conjugate, such as a tocopherol ester, for example, tocopherol succinate. The esterification reaction can be performed by any of a number of known methods, including those described in U.S. Pat. Nos. 2,680,749; 4,665,204; 3,538,119; and 6,632,443. The resulting vitamin E-linker conjugate can then be attached to a PEG moiety by another esterification reaction, for example, between a carboxylic acid group of the vitamin E-linker conjugate and a hydroxyl group of the PEG moiety, to form a vitamin E-PEG derivative.

PEG derivatives of a tocopherol-linker or tocotrienol-linker compound can be made by any other method known to those of skill in the art. Various methods known in the art for producing PEG derivatives can be used to attach a PEG molecule to tocopherol-linker or tocotrienol-linker compounds. For example, a tocopherol-linker compound can form a covalent bond to the PEG molecule via an amide, ether or thioether bond. For example, a tocopherol-linker conjugate that contains an amine group can be reacted with a PEG-NHS (N-hydroxysuccinimide) derivative to form an amide bond between the tocopherol-linker conjugate and the PEG molecule. A tocopherol-linker conjugate that contains an amine group can be reacted with a PEG-aldehyde derivative to form an amide bond between the tocopherol-linker conjugate and the PEG molecule. In another example, a tocopherol-linker conjugate that contains an carboxylic acid can be activated to the corresponding acid halide and reacted with a PEG-SH derivative to form a thioester bond between the tocopherol-linker conjugate and the PEG molecule.

a) Tocopherols and Tocotrienols

The vitamin E derivative can be any vitamin E derivative, for example, any tocopherol or tocotrienol. The tocopherols used to make the surfactant can be any natural or synthetic vitamin E tocopherol, including, but not limited to, alpha-tocopherols, beta-tocopherols, gamma-tocopherols, and delta tocopherols, either in pure forms or in heterogenous mixtures of more than one form. Exemplary tocopherols are d-α-tocopherols and dl-tocopherols. To make the surfactant, the tocopherol typically is esterified with a linker, for example, a dicarboxylic acid, to form a tocopherol ester, which then is joined to a PEG moiety.

The tocotrienols used to make the surfactants can be any natural or synthetic vitamin E tocotrienol, including, but not limited to, alpha-tocotrienols, beta-tocotrienols, gamma-tocotrienols, and delta tocotrienols, either in pure forms or in heterogenous mixtures of more than one form. Mixtures of tocopherols and tocotrienols are contemplated for use in the provided methods and compositions. A tocotrienol can be esterified with a linker, such as a dicarboxylic acid, before joining with a PEG moiety.
b) Linkers

Typically, the PEG derivatives of vitamin E are diesters or other esters, e.g., triesters. When the PEG derivative is a diester, the linker joining the vitamin E moiety to the PEG typically is a carboxylic acid, typically a dicarboxylic acid, as in, for example, tocopherol polyethylene glycol succinate (TPGS), where the linker is a succinic acid, and the surfactant is made by an esterification reaction joining a PEG moiety and a tocopherol ester of the dicarboxylic acid. In another example, the linker is another molecule, for example, an amino acid, such as glycine, alanine, 5-aminopentanoic acid or 8-aminooctanoic acid, or the linker is an amino alcohol, such as ethanolamine.

c) PEG Moieties

The polyalkylene glycol moiety used in the polyalkylene glycol vitamin E derivative can be any of a plurality of known polyalkylene glycol moieties, such as any known PEG moiety. Exemplary of suitable polyalkylene glycol moieties are for example, PEG moieties, such as PEG moieties having varying chain lengths, and varying molecular weights, for example, PEG 1000, PEG 200, PEG 500, and PEG 20,000. The number following the individual PEG moiety indicates the molecular weight (in daltons (Da) of the PEG moiety. Typically, the PEG moiety of a tocopherol-derived surfactant has a molecular weight of between 200 or about 200 to 20,000 or about 20,000 Da, typically between 200 and 6000 Da, for example, between 600 or about 600 Da and 6000 or about 6000 Da, typically between 200 or about 200 Da and 2000 or about 2000 Da, between 600 or about 600 Da and 1500 or about 1500 Da, such as 200, 300, 400, 500, 600, 800, and 1000 Da. Exemplary of a PEG derivative of a tocopherol ester having a PEG moiety with a molecular weight of 1000 Da is TPGS-1000. Also exemplary of suitable PEG moieties are PEG moieties that are modified, for example, methylated PEG (m-PEG), which is a PEG chain capped with a methyl group. Other known PEG analogs also can be used. The PEG moieties can be selected from among any reactive PEG, including, but not limited to, PEG-OH, PEG-NHS, PEG-aldehyde, PEG-SH, PEG-NH₂, PEG-COOH, and branched PEGs.

2) Tocopheryl Polyalkylene Glycol Derivatives

In its natural water-insoluble state, vitamin E, e.g., tocopherol or tocotrienol, is easily absorbed and used in humans and animals. Processing of foods and feeds by industry for long-term storage can promote accelerated degradation of the effective vitamin E content. To compensate for the loss of natural vitamin E from food sources, nutritional supplements of natural or synthetic fat-soluble vitamin E have been developed. Not all humans and animals can sufficiently absorb the supplements though. To address this problem, water-soluble vitamin E derivatives have been developed that are an excellent source of vitamin E (i.e., maintain a high degree of vitamin E biological activity) in humans with impaired vitamin E absorption, for example, in humans with malabsorption syndromes (Traber et al. (1986) Am. J. Clin. Nutr. 44:914-923). Water-soluble vitamin E derivatives have been developed for this purpose. The water-soluble vitamin E derivative D-α-tocopheryl polyethylene glycol succinate (TPGS) is exemplary of the tocopheryl polyethylene glycol derivatives.

TPGS contains a hydrophilic (i.e., water-soluble) polyethylene glycol (PEG) chain and a lipophilic (i.e., water-insoluble) α-tocopherol head. The amphiphilic structure of TPGS, shown below, renders it much more water-soluble than traditional vitamin E, allowing TPGS to form a micellar solution at low concentrations (0.04-0.06 mmol/L) that can be absorbed by humans and animals in the absence of bile salts.

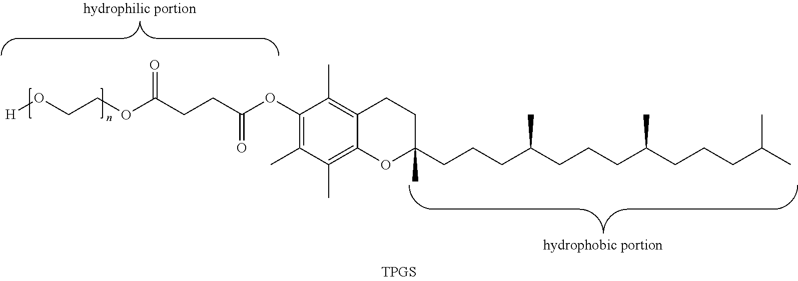

TPGS has been approved by the FDA as a water-soluble vitamin E nutritional supplement. It is a GRAS (Generally Regarded As Safe)-listed supplement that can be taken orally at long-term doses of 13.4-16.8 mg/kg/day or up to 100 mg/kg/day for people with impaired uptake. In the body, TPGS undergoes enzymatic cleavage to deliver the lipophilic antioxidant α-tocopherol (vitamin E) to cell membranes. Cellular enzymatic hydrolysis by cytoplasmic esterases liberates free α-tocopherol, which then localizes in the cell membrane, and through free radical quenching, protects the membrane from lipid peroxidation and damage.

TPGS also is used as a non-ionic surfactant and emulsifier that, as reported, has an HLB value of approximately 13. Non-ionic surface-active agents are used in oral formulations to enhance the bioavailability of water-insoluble pharmaceuticals, such as drugs, vitamins, or other biologically active compounds. TPGS is an effective absorption and bioavailability enhancer, and has been approved for use as a drug solubilizer in oral, parenteral, topical, nasal, and rectal/vaginal therapies (see, e.g., Constantinides et al. (2006) Pharm. Res. 23(2):243-255; Varma et al. (2005) Eur. J. Pharm. Sci. 25(4-5):445-453) and as a solubilizer for inhalation drug delivery (Fulzele et al. (2006) 23(9):2094-2106). TPGS improves the bioavailability of such water-insoluble drugs as the HIV protease inhibitor amprenavir (Yu et al. (1999) Pharm. Res. 16:1812-1817; Brouwers et al. (2006) J. Pharm. Sci. 95:372-383), the non-nucleoside reverse transcriptase inhibitor UC 781 (Goddeeris et al. (2008) Eur. J. Pharm. Sci. 35:104-113), cyclosporin (Sokol et al. (1991) Lancet 338:212-215), paclitaxel (Zhao et al. (2010) J. Pharm. Sci. 99(8):3552-3560), estradiol (Sheu et al. (2003) J. Controlled Release 88:355-368), and fat-soluble vitamins such as vitamin D (Argao et al. (1992) Ped. Res. 31(2):146-150).

Exemplary of a tocopheryl polyalkylene glycol derivative that can be included in the pre-gel concentrates provided herein is D-α-tocopheryl polyethylene glycol succinate (TPGS), such as TPGS-1000, for example, the food grade TPGS sold under the name Eastman Vitamin E TPGS®, food grade, by Eastman Chemical Company, Kingsport, Tenn. Other exemplary tocopheryl polyalkylene glycol derivatives suitable for use in the pre-gel concentrates provided herein are tocopheryl polyalkylene glycol compositions, for example, TPGS compositions, containing a relatively high percentage, such as at least 13%, typically at least 20%, 25%, 29%, 30%, 35%, 40%, 45%, 48%, 49%, 50%, or more, typically up to 60-65%, of the dimer form of TPGS, with the remainder of the TPGS composition containing the monomer form of TPGS and a small percentage, such as less than 5%, 4%, 3%, 2%, 1% of contaminants, such as higher order polymers and reagents, such as vitamin E and polyethylene glycol. Exemplary of tocopheryl polyalkylene glycol derivatives are those described in U.S. patent application Ser. No. 14/207,310 and International PCT Application No. PCT/US14/25006, now published as US-2014-0271593-A1 and WO 2014/151109, respectively, both of which are incorporated herein by reference in their entirety.

a) Synthesis

Scheme 1 shows the synthesis of an exemplary water-soluble vitamin E derivative, TPGS, but any vitamin E moiety, i.e., any tocopherol or tocotrienol, can be used as the starting material and reacted with any linker, such as those described herein, that is capable of reacting with a polyalkylene glycol moiety to form a monomer form and dimer form of a water-soluble vitamin E derivative. As shown in Scheme 1 below, TPGS can be prepared by reacting vitamin E with succinic anhydride or succinic acid to obtain vitamin E succinate, i.e., D-α-tocopheryl succinate, followed by esterification with a polyethylene glycol molecule, to obtain TPGS (see U.S. Pat. No. 2,680,749). TPGS analogs varying in PEG chain length (e.g., TPGS 200, 238, 400, 600, 2000, 3400, 3500, 4000 and 6000) have been synthesized, but the most widely used form of TPGS is TPGS 1000, which incorporates PEG 1000, a polyethylene glycol molecule with a molecular weight of approximately 1,000 Daltons (Collnot et al. (2006) J. Controlled Release 111:35-40). TPGS 1000 is a pale yellow, waxy solid substance that is amphipathic and hydrophilic, with a molecular weight of approximately 1,513 Daltons.

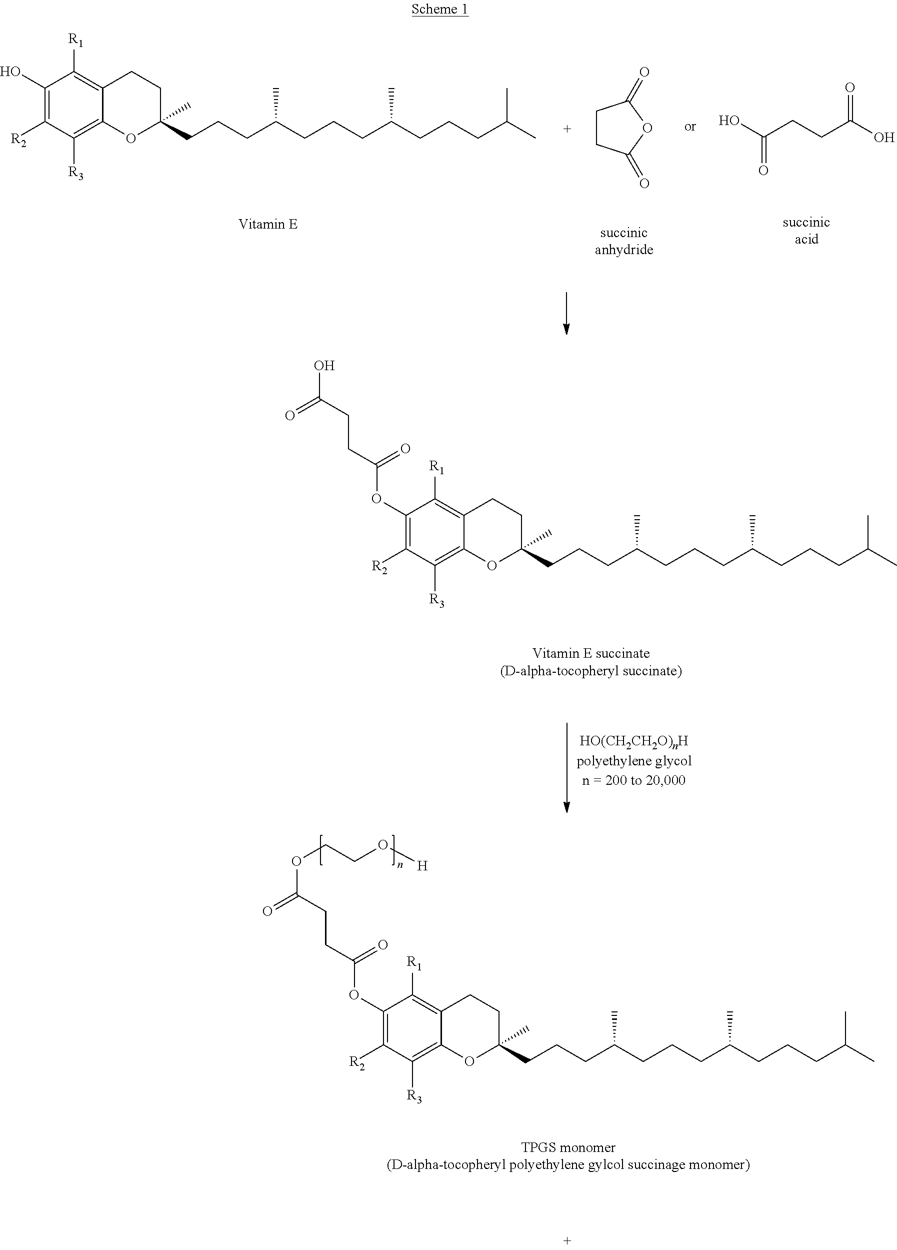

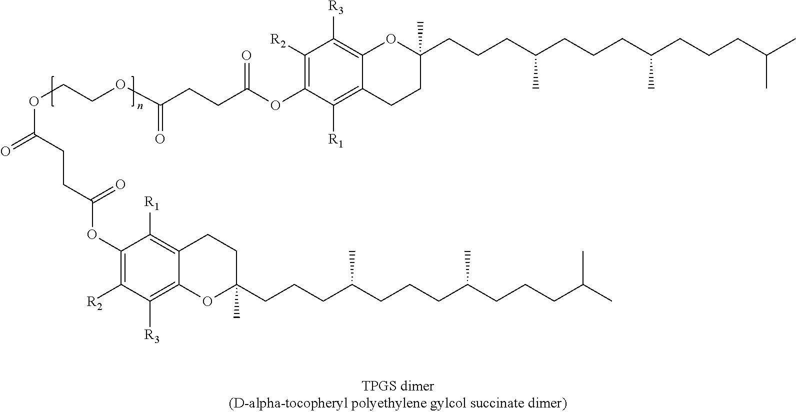

TPGS compositions, as generally prepared, such as commercially available TPGS 1000, are mixtures that contain primarily TPGS monomer (between 70% and 87% or more) and a lesser amount of TPGS dimer (less than 12%). The monomer is considered the effective component in TPGS, while the dimer is viewed as a byproduct of the esterification reaction between polyethylene glycol and vitamin E succinate. For example, commercially available TPGS, such as the TPGS 1000 available from Eastman Chemical Company (Kingsport, Tenn.), contains primarily TPGS monomer (˜86% or more) and a small amount of TPGS dimer (˜11% or less) (Christiansen et al. (2011) J. Pharm. Sci. 100(5):1773-1782). TPGS synthesized according to standard methods, for example, the method described in U.S. Pat. No. 2,680,749, results in a TPGS composition that is composed primarily of TPGS monomer (70-87%) and a small amount of TPGS dimer (<12%) (US Pharmacopeia 23 (1998) Supp. 9:4712; Scientific Panel of the European Food Safety Authority (2007) EFSA J. 490:1-20). Because the separation of TPGS monomer and TPGS dimer is difficult and because TPGS monomer is considered the effective component of TPGS, TPGS compositions containing primarily TPGS dimer have not been developed (Kong et al. (2011) J. Chromatography A 1218:8664-8671). TPGS dimer, shown below, is usually considered an unwanted byproduct of the esterification reaction between PEG and vitamin E succinate, formed due to the equal reactivity of both terminal hydroxyl groups of the PEG moiety.

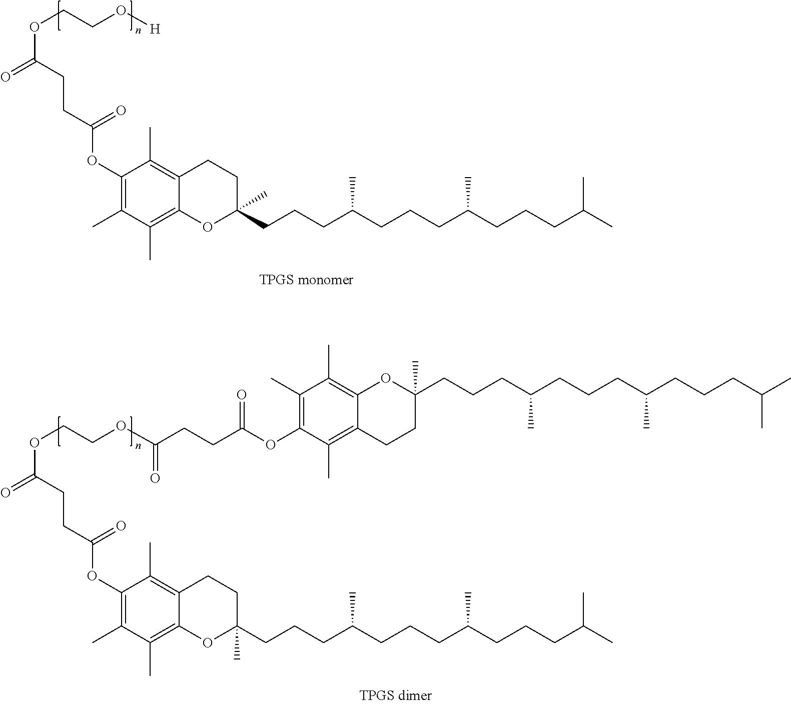

b) Water-Soluble Vitamin E Derivative Mixtures (Compositions)

The water-soluble vitamin E derivative mixtures (compositions), for example, TPGS compositions, that can be used in soft-gels and pre-gels provided herein can contain varying amounts of monomer and dimer, particularly TPGS compositions that contain less monomer than is found in typical, known water-soluble vitamin E derivative mixtures (compositions), for example, less than 70 wt % monomer, and more dimer, i.e., greater than 12 wt % dimer, than in typical, known water-soluble vitamin E derivative mixtures (compositions), for example, known TPGS compositions. For example, the water-soluble vitamin E derivative mixtures (compositions) can be high dimer mixtures and can contain, for example, between or between about 25 wt % and 69 wt % monomer and between or between about 13 wt % and 95 wt % dimer, such as water-soluble vitamin E derivative mixtures (compositions) containing between or about between 40 wt % and 60 wt % monomer and between or about between 25 wt % and 60 wt % dimer, such as 29% to 55%, 35% to 50% or 30% to 45%, dimer. Advantageous properties are exhibited by soft-gels and pre-gels that contain TPGS compositions with at least these amounts.

In the water-soluble vitamin E derivative mixtures (compositions) that can be used in the soft-gels and pre-gels described herein, the total amount of monomer as a percentage (%) by weight of the water-soluble vitamin E derivative mixture (composition) (wt %) can be, e.g., between or between about 25 wt % and 69 wt % monomer, inclusive, such as between or between about 25% and 30%, 25% and 35%, 25% and 40%, 25% and 45%, 25% and 50%, 25% and 55%, 25% and 60%, 25% and 65%, 25% and 69%, 30% and 35%, 30% and 40%, 30% and 45%, 30% and 50%, 30% and 55%, 30% and 60%, 30% and 65%, 30% and 69%, 35% and 40%, 35% and 45%, 35% and 50%, 35% and 55%, 35% and 60%, 35% and 65%, 35% and 69%, 40% and 45%, 40% and 50%, 40% and 55%, 40% and 60%, 40% and 65%, 40% and 69%, 45% and 50%, 45% and 55%, 45% and 60%, 45% and 65%, 45% and 69%, 50% and 55%, 50% and 60%, 50% and 65%, 50% and 69%, 55% and 60%, 55% and 65%, 55% and 69%, 60% and 65%, 60% and 69%, and 65% and 69% monomer, by weight of the composition. Generally, the water-soluble vitamin E derivative mixtures (compositions) contain less than 69 wt % monomer. For example, the water-soluble vitamin E derivative mixtures (compositions) that can be used in the soft-gels and pre-gels described herein contain at least or about at least 25%, 30%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, but less than 69% (wt %), total monomer.

In the water-soluble vitamin E derivative mixtures (compositions) that can be used in the soft-gels and pre-gels described herein, the total amount of dimer as a percentage (%) by weight of the water-soluble vitamin E derivative mixture (composition) (wt %) can be, e.g., between or between about 13 wt % and 95 wt % dimer, inclusive, such as between or between about 13% and 20%, 13% and 25%, 13% and 30%, 13% and 35%, 13% and 40%, 13% and 45%, 13% and 50%, 13% and 55%, 13% and 60%, 13% and 65%, 13% and 70%, 13% and 75%, 13% and 80%, 13% and 85%, 13% and 90%, 13% and 95%, 20% and 25%, 20% and 30%, 20% and 35%, 20% and 40%, 20% and 45%, 20% and 50%, 20% and 55%, 20% and 60%, 20% and 65%, 20% and 70%, 20% and 75%, 20% and 80%, 20% and 85%, 20% and 90%, 20% and 95%, 25% and 30%, 25% and 35%, 25% and 40%, 25% and 45%, 25% and 50%, 25% and 55%, 25% and 60%, 25% and 65%, 25% and 70%, 25% and 75%, 25% and 80%, 25% and 85%, 25% and 90%, 25% and 95%, 30% and 35%, 30% and 40%, 30% and 45%, 30% and 50%, 30% and 55%, 30% and 60%, 30% and 65%, 30% and 70%, 30% and 75%, 30% and 80%, 30% and 85%, 30% and 90%, 30% and 95%, 35% and 40%, 35% and 45%, 35% and 50%, 35% and 55%, 35% and 60%, 35% and 65%, 35% and 70%, 35% and 75%, 35% and 80%, 35% and 85%, 35% and 90%, 35% and 95%, 40% and 45%, 40% and 50%, 40% and 55%, 40% and 60%, 40% and 65%, 40% and 70%, 40% and 75%, 40% and 80%, 40% and 85%, 40% and 90%, 40% and 95%, 45% and 50%, 45% and 55%, 45% and 60%, 45% and 65%, 45% and 70%, 45% and 75%, 45% and 80%, 45% and 85%, 45% and 90%, 45% and 95%, 50% and 55%, 50% and 60%, 50% and 65%, 50% and 70%, 50% and 75%, 50% and 80%, 50% and 85%, 50% and 90%, 50% and 95%, 55% and 60%, 55% and 65%, 55% and 70%, 55% and 75%, 55% and 80%, 55% and 85%, 55% and 90%, 55% and 95%, 60% and 65%, 60% and 70%, 60% and 75%, 60% and 80%, 60% and 85%, 60% and 90%, 60% and 95%, 65% and 70%, 65% and 75%, 65% and 80%, 65% and 85%, 65% and 90%, 65% and 95%, 70% to 75%, 70% and 80%, 70% and 85%, 70% and 90%, 70% and 95%, 75% and 80%, 75% and 85%, 75% and 90%, 75% and 95%, 80% and 85%, 80% and 90%, 80% and 95%, 85% and 90%, 85% and 95% and 90% and 95% dimer, by weight of the water-soluble vitamin E derivative mixture (composition). Generally, the water-soluble vitamin E derivative mixtures (compositions) contain less than 95 wt % dimer. For example, the water-soluble vitamin E derivative mixtures (compositions) that can be used in the soft-gels and pre-gels described herein contain at least or about at least 13%, 15%, 20%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, but less than 95% (wt %) total dimer.

The water-soluble vitamin E derivative mixtures (compositions) that can be used in the soft-gels and pre-gels described herein that contain less than 70 wt % monomer and greater than 12 wt % dimer (e.g., high dimer mixtures) exhibit decreased turbidity values when dissolved in an aqueous solution, for example, when dissolved in water, as compared to typical, known water-soluble vitamin E derivative mixtures (compositions), i.e., water-soluble vitamin E derivative mixtures (compositions) that contain more than 70 wt % monomer and less than 12 wt % dimer. The compositions containing less than 70 wt % monomer and greater than 12 wt % dimer allow for the addition of a higher concentration of non-polar ingredients when used in aqueous food and beverage products as compared to available aqueous food and beverage products, while maintaining clarity and stability, for example, exhibiting decreased turbidity values.

Exemplary of the compositions are TPGS compositions containing less than 70 wt % TPGS monomer and more than 12 wt % TPGS dimer (e.g., high dimer mixtures), such as compositions containing between or about between 25 wt % and 69 wt % TPGS monomer and between or about between 13 wt % and 95 wt % TPGS dimer, such as TPGS compositions containing between or about between 40 wt % and 60 wt % TPGS monomer and between or about between 25 wt % and 60 wt % TPGS dimer, are described herein. The compositions containing less than 70 wt % TPGS monomer and greater than 12 wt % TPGS dimer exhibit decreased turbidity values when dissolved, for example, when dissolved in water, as compared to typical, known TPGS compositions, i.e., TPGS compositions that contain more than 70 wt % TPGS monomer and less than 12 wt % TPGS dimer. The TPGS compositions allow for the addition of a higher concentration of non-polar ingredients when used in aqueous food and beverage products as compared to available aqueous food and beverage products, while maintaining clarity and stability, for example, exhibiting decreased turbidity values.

The water-soluble vitamin E derivative mixtures (compositions), e.g., TPGS compositions, that can be used in the soft-gels and pre-gels described herein contain a mixture of monomer and dimer, e.g., a mixture of TPGS monomer and TPGS dimer. The monomer, for example, a TPGS monomer, can be present in an amount that is less than what is typically found in known water-soluble vitamin E derivative mixtures (compositions), e.g., known TPGS compositions, i.e., less than 70 wt % monomer. The dimer, for example, a TPGS dimer, can be present in an amount that is greater than what is typically found in known water-soluble vitamin E derivative mixtures (compositions), e.g., known TPGS compositions, i.e., greater than 12 wt % dimer. The water-soluble vitamin E derivative mixtures (compositions), such as the TPGS compositions, can also contain other components, such as, for example, unreacted PEG, unreacted vitamin E, e.g., D-α-tocopheryl succinate, and one or more catalysts.

Methods for preparing the water-soluble vitamin E derivative mixtures (compositions), such as the TPGS compositions that can be used in the soft-gels and pre-gels described herein, are described herein, for example, methods of preparing water-soluble vitamin E derivative compositions, such as TPGS compositions, that contain less than 70 wt % TPGS monomer and more than 12 wt % TPGS dimer (e.g., high dimer mixtures). Existing methods for preparing derivatives of vitamin E can be employed, except that the methods are modified to produce higher concentrations of the dimer form by modifying reaction conditions. Such modifications can be determined empirically if needed, such as by varying reaction parameters, such as time, temperature and reactant concentrations, to identify conditions that favor higher levels of dimer production.

The water-soluble vitamin E derivative mixtures e.g., TPGS monomer-dimer mixtures, prepared according to the methods, can contain between or about between 25 wt % and 69 wt % monomer, for example, at or about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69 wt % monomer and between or about between 13 wt % and 95 wt % dimer, for example, at or about 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 89, 80, 81, 82, 83, 84, 85, 86, 87, 88, 89, 90, 91, 92, 93, 94 or 95 wt % dimer.

Exemplary of the water-soluble vitamin E derivative mixtures (compositions) that can be used in the soft-gels and pre-gels described herein that contain a mixture of monomer and dimer, for example, TPGS compositions that contain a mixture of TPGS monomer and TPGS dimer, are compositions that contain between or about between 25 wt % and 69 wt % monomer and between or about between 13 wt % and 95 wt %, such as 29% to 55%, dimer. Thus, described herein are water-soluble vitamin E derivative mixtures (compositions), such as TPGS compositions, such as high dimer TPGS compositions, that contain less monomer, i.e., less than 70 wt % monomer, such as between 25 wt % and 69 wt % monomer, and more dimer, i.e., more than 12 wt % dimer, such as between 13 wt % and 95% dimer, than typical commercial TPGS compositions.

3) Methods for Making Water-Soluble Vitamin E Derivatives

The water-soluble vitamin E derivative mixtures (compositions) with higher amounts of dimer can be prepared by modification of methods that compositions with higher amounts of monomer and lower amounts of dimer are prepared by, appropriately varying reaction conditions to favor increased dimer formation. Alternatively, standard known methods can be employed and the dimers purified or partially purified and added to compositions to increase the percentage of dimer to a desired level.

For example, for production of compositions with higher amounts of TPGS dimer, the methods employ the use of vitamin E succinate, e.g., D-α-tocopheryl succinate, as a starting material. Methods that use vitamin E, e.g., tocopherol or tocotrienol, and succinic acid or succinic anhydride as the starting materials (to synthesize vitamin E succinate) also can be used to prepare the water-soluble vitamin E derivative mixtures (compositions) described herein. The methods can be adapted for production of any desired water-soluble vitamin E derivative composition that contains the higher amounts of dimer.

As noted, these water-soluble vitamin E derivative mixtures (compositions) exhibit decreased turbidity values as compared to known water-soluble vitamin E derivative mixtures (compositions), such as known TPGS compositions, when dissolved, such as, for example, when dissolved in water or other aqueous beverages. Thus, the described methods are advantageous over existing prior art methods of preparing TPGS compositions that exhibit high turbidity values, e.g., higher than 80 NTUs, when dissolved, such as when dissolved in water.

Water-soluble vitamin E derivatives, such as TPGS, can be prepared by esterifying vitamin E succinate, for example, D-α-tocopheryl acid succinate, with polyethylene glycol. The resulting vitamin E TPGS has a chemical formula of C₃₃O₅H₅₄(CH₂CH₂O)ₙ, where “n” represents the number of polyethylene oxide moieties attached to the acid group of the vitamin E succinate. In an exemplary embodiment, the method includes preparing a crude water-soluble vitamin E, e.g., TPGS, composition by first preparing a reaction mixture containing vitamin E succinate, a polyethylene glycol (PEG), and optionally, a catalyst, in a solvent, and heating the reaction mixture to an elevated temperature to produce a crude water-soluble vitamin E, e.g., TPGS, composition containing less TPGS monomer and more TPGS dimer than what is typically found in known TPGS compositions, i.e., less than 70 wt % TPGS monomer and more than 12 wt % TPGS dimer. The crude water-soluble vitamin E, e.g., TPGS, composition then can be purified and concentrated to obtain a purified water-soluble vitamin E, e.g., TPGS, composition containing less TPGS monomer and more TPGS dimer than what is typically found in known TPGS compositions, i.e., less than 70 wt % TPGS monomer and more than 12 wt % TPGS dimer. Any purification process known in the art can be used to purify the reaction product.

a) Reaction Mixture

The water-soluble vitamin E derivative mixtures can be prepared by first preparing a crude water-soluble vitamin E derivative mixture, such as a crude TPGS composition, by esterifying vitamin E succinate with polyethylene glycol in a solvent. The esterification procedure can be promoted by a catalyst, for example, an esterification catalyst. The crude composition can be prepared from a reaction mixture containing vitamin E succinate, a polyethylene glycol (PEG), a solvent, and optionally, a catalyst. The components of the reaction mixture can be added in any order. In an exemplary embodiment, the polyethylene glycol is dissolved in the solvent before the addition of vitamin E succinate and the catalyst.

A crude water-soluble vitamin E derivative mixture, such as a crude TPGS composition, that contains less TPGS monomer and more TPGS dimer than what is typically found in known TPGS compositions, i.e., less than 70 wt % TPGS monomer and more than 12 wt % TPGS dimer can be produced. In some instances, the crude TPGS composition contains between or about between 25 wt % and 69 wt % TPGS monomer and between or about between 13 wt % and 95 wt % TPGS dimer, such as between or about between 40 wt % and 60 wt % TPGS monomer and between or about between 25 wt % and 60 wt % TPGS dimer.

i. Vitamin E Succinate

The reaction mixtures can contain vitamin E succinate, for example, D-α-tocopheryl succinate. Vitamin E succinate can be purchased from suppliers such as Sigma-Aldrich (St. Louis, Mo.), Parchem (New Rochelle, N.Y.), Fisher Scientific (Fair Lawn, N.J.), and VWR International (Radnor, Pa.), or can be synthesized according to methods known to those of skill in the art. Typically, vitamin E succinate can be synthesized by reacting vitamin E (i.e., D-α-tocopherol) with succinic anhydride in a solvent (e.g., toluene) in the presence of a base (e.g., triethylamine) (see, for example, U.S. Patent Pub. Nos. 2011/0130562 and 2011/0184194; Lipshutz et al. (2011) J. Org. Chem. 76(11):4379-4391; Gelo-Pujic et al. (2008) Int. J. Cosmet. Sci. 30(3):195-204; and Vraka et al. (2006) Bioorg. Med. Chem. 14(8):2684-2696).

The total amount of vitamin E succinate in the reaction mixture as a percentage (%) by weight of the reaction mixture (wt %) can be, e.g., from at or about 0.1% to at or about 15%, such as 0.1% to 1%, 0.1% to 3%, 0.1% to 5%, 0.1% to 10%, 0.1% to 15%, 0.5% to 1%, 0.5% to 3%, 0.5% to 5%, 0.5% to 10%, 0.5% to 15%, 1% to 3%, 1% to 5%, 1% to 10%, 1% to 15%, 3% to 5%, 3% to 10%, 3% to 15%, 5% to 10%, 5% to 15%, or 10% to 15% by weight of the reaction mixture. Generally, the reaction mixtures contain less than 15 wt % vitamin E succinate. For example, the reaction mixtures described herein contain up to at or about 1%, 2%, 3%, 4%, 5%, 6%, 7%, 8%, 9%, 10%, 11%, 12%, 13%, 14% or 15% vitamin E succinate. Generally, the reaction mixtures described herein contain less than 15% (wt %) total vitamin E succinate.

ii. Polyethylene Glycol

The reaction mixtures include any polyethylene glycol that can react with the acid moiety of vitamin E succinate to form an ester. The polyethylene glycol can include, for example, any polyethylene glycol that gives the desired molecular weight of the water-soluble vitamin E compound, the desired polyethylene glycol chain length of the water-soluble vitamin E compound or the desired amount of water-soluble vitamin E water-solubility. The polyethylene glycol in the reaction mixtures can include, for example, any polyethylene glycol that is capable of forming an ester when reacted with vitamin E succinate to produce a vitamin E derivative that is water-soluble. For example, the polyethylene glycol can include PEG-OH, PEG-SH, PEG-NH₂ and branched PEGs. Typically, the polyethylene glycol is PEG-OH. The resulting water-soluble vitamin E product, for example, TPGS, formed by the reaction between vitamin E succinate and a polyethylene glycol contains at least polyethylene glycol esters of vitamin E succinate. The esters can be a mixture of esters, such as a mixture of TPGS monomer and TPGS dimer.

The polyethylene glycols in the reaction mixtures can be any molecular weight, for example, any molecular weight that renders vitamin E succinate water-soluble after esterification with the polyethylene glycol (i.e., the resulting TPGS is water-soluble). Such polyethylene glycols are known in the art and can be purchased from suppliers such as Sigma-Aldrich (St. Louis, Mo.), Fisher Scientific (Fair Lawn, N.J.), and VWR International (Radnor, Pa.). The polyethylene glycol can be added to the reaction mixture by any method suitable for transferring the PEG to the reaction mixture. For example, the PEG can be transferred to the reaction mixture in molten form.

Suitable polyethylene glycols include polyethylene glycols having an average molecular weight ranging from between or about between 100 Daltons (Da) and 20,000 Da. For example, the average molecular weight can be between or about between 200 Da and 10,000 Da, or 400 Da and 5,000 Da, or 500 Da and 1500 Da, or 750 Da and 1200 Da, or 1000 Da and 2,500 Da. Generally, the molecular weight of the polyethylene glycol is less than 20,000 Da. For example, the average molecular weight of the polyethylene glycol used in the reaction mixtures can be or can be about 100, 200, 238, 300, 400, 500, 600, 750, 800, 1000, 1200, 1500, 2000, 2500, 3000, 3400, 3500, 4000, 6000, 8000, 10,000, or 12,000 Da, but less than 20,000 Da.

Exemplary polyethylene glycols include PEG 100 (where 100 represents the PEG chain molecular weight), PEG 200, PEG 238, PEG 300, PEG 400, PEG 500, PEG 600, PEG 750, PEG 800, PEG 1000, PEG 1200, PEG 1500, PEG 2000, PEG 2500, PEG 3000, PEG 3400, PEG 3500, PEG 4000, PEG 6000, PEG 8000, PEG 10,000, PEG 12,000 or PEG 20,000. Any other suitable polyethylene glycol known to those of skill in the art also can be used in the methods. In some embodiments described herein, the polyethylene glycol is PEG 1000.

The total amount of PEG in the reaction mixture as a percentage (%) by weight of the reaction mixture (wt %) can be, e.g., from at or about 1% to at or about 50%, such as 1% to 5%, 5% to 10%, 5% to 15%, 5% to 20%, 5% to 25%, 5% to 30%, 5% to 35%, 5% to 40%, 5% to 45%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 40%, 20% to 50%, 25% to 50%, or 30% to 50% by weight of the reaction mixture. Generally, the reaction mixtures contain less than 50 wt % PEG For example, the reaction mixtures described herein contain at least or about at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, but less than 50% (wt %) total PEG

iii. Catalyst

The reaction mixtures can optionally contain a catalyst. Suitable catalysts include those catalysts that can be used to promote the esterification reaction between the PEG and the acid moiety of vitamin E succinate. Exemplary catalysts include acidic catalysts, such as p-toluenesulfonic acid, oxalic acid, hydrochloric acid, tricholoracetic acid, and any other known catalyst that can promote esterification.

In the reaction mixtures, the total amount of catalyst, as a percentage (%) by weight of the reaction mixture (wt %) can be, e.g., from at or about 0% to at or about 15%, such as 0.01% to 0.05%, 0.01% to 0.1%, 0.01% to 0.5%, 0.01% to 0.75%, 0.01% to 1%, 0.01% to 3%, 0.01% to 5%, 0.01% to 10%, 0.01% to 15%, 0.01% to 0.5%, 0.01% to 0.75%, 0.01% to 1%, 0.01% to 3%, 0.01% to 5%, 0.01% to 10%, 0.01% to 15%, 0.05% to 0.1%, 0.05% to 0.5%, 0.05% to 0.75%, 0.05% to 1%, 0.05% to 3%, 0.05% to 5%, 0.05% to 10%, 0.05% to 15%, 0.05% to 0.5%, 0.05% to 0.75%, 0.05% to 1%, 0.05% to 3%, 0.05% to 5%, 0.05% to 10%, 0.05% to 15%, 0.1% to 0.5%, 0.1% to 0.75%, 0.1% to 1%, 0.1% to 3%, 0.1% to 5%, 0.1% to 10%, 0.1% to 15%, 0.5% to 0.75%, 0.5% to 1%, 0.5% to 3%, 0.5% to 5%, 0.5% to 10%, 0.5% to 15%, 1% to 3%, 1% to 5%, 1% to 10%, 1% to 15%, 3% to 5%, 3% to 10%, 3% to 15%, 5% to 10%, 5% to 15%, 10% to 15% by weight of the reaction mixture. Generally, the reaction mixtures contain less than 15 wt % catalyst. For example, the reaction mixtures described herein can contain up to at or about 1%, 2%, 3%, 4%, 5%, 6%, 7%, 8%, 9%, 10%, 11%, 12%, 13%, 14% or 15% catalyst, based on the weight of the reaction mixture.

iv. Solvent

The reaction mixtures include a solvent or combination of solvents. Suitable solvents include those that do not prevent the esterification reaction between the PEG and acid moiety of vitamin E succinate from taking place. For example, the solvent or combination of solvents can be aprotic solvents.

Suitable solvents include solvents that are inert to the reaction and are aprotic, for example, solvents that lack an acidic hydrogen, such as toluene, xylenes, ethers such as tetrahydrofuran (THF), diethyl ether and dioxane, ethyl acetate, acetone, dimethylformamide (DMF), N,N-dimethylacetamide, acetonitrile, methyl ethyl ketone (MEK), methyl isobutyl ketone (MIBK), dimethyl sulfoxide (DMSO), ethyleneglycol dimethylether, hexanes, cyclohexane, pentane, cyclopentane and any combination thereof. An exemplary solvent used in the reaction mixtures is toluene.

In the reaction mixtures, the total amount of solvent as a percentage (%) by weight of the reaction mixture (wt %) can be, e.g., from at or about 60% to at or about 95%, such as 60% to 65%, 60% to 70%, 60% to 75%, 60% to 80%, 60% to 85%, 60% to 90%, 60% to 95%, 65% to 70%, 65% to 75%, 65% to 80%, 65% to 85%, 65% to 90%, 65% to 95%, 70% to 75%, 70% to 80%, 70% to 85%, 70% to 90%, 70% to 95%, 75% to 80%, 75% to 85%, 75% to 90%, 75% to 95%, 80% to 85%, 80% to 90%, 80% to 95%, 85% to 90%, 85% to 95% and 90% to 95%, by weight of the reaction mixture. Generally, the reaction mixtures contain less than 95 wt % solvent. For example, the reaction mixtures can contain at least or about at least 60%, 65%, 70%, 75%, 80%, 85%, 90%, but less than 95% (wt %) total solvent.

v. Exemplary Reaction Mixtures

Exemplary reaction mixtures that can be used to ultimately produce a water-soluble vitamin E derivative mixture, for example, a TPGS composition, that contains less TPGS monomer and more TPGS dimer than what is typically manufactured, i.e., less than 70 wt % TPGS monomer and more than 12 wt % TPGS dimer, are described. They are exemplified with TPGS, but similar reaction mixtures can be prepared and reactions performed to produce tocopherol sebacate polyethylene glycol, tocopherol dodecanodioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol and tocopherol phthalate polyethylene glycol, TPGS analogs and TPGS homologs.

The reaction mixtures exemplified herein include vitamin E succinate, a polyethylene glycol, a solvent, and optionally, a catalyst. Exemplary of such reaction mixtures contain from at or about 0.1 wt % to at or about 15 wt % of vitamin E succinate; a polyethylene glycol, in an amount from at or about 1 wt % to at or about 50 wt %; a catalyst, in an amount from at or about 0.01 wt % to at or about 15 wt %; and from at or about 60% to at or about 95% of a solvent.

In some embodiments, the polyethylene glycol can be a polyethylene glycol with a molecular weight of around 1000 Da, for example, PEG 1000. For example, the exemplary reaction mixtures described herein can contain from at or about 0.1 wt % to at or about 15 wt % of vitamin E succinate; from at or about 1 wt % to at or about 50 wt % of a polyethylene glycol, for example, PEG 1000; from at or about 0.01 wt % to at or about 15 wt % of a catalyst, for example, p-toluenesulfonic acid; and from at or about 60% to at or about 95% of a solvent, for example, toluene.

b) Exemplary Methods

The methods include preparing a reaction mixture containing vitamin E succinate, a polyethylene glycol and optionally, a catalyst, in a solvent; heating the reaction mixture to a temperature equal to or higher than the boiling point of the solvent to form a crude water-soluble vitamin E derivative mixture; processing the reaction mixture to obtain the crude water-soluble vitamin E derivative mixture; and purifying the crude water-soluble vitamin E derivative mixture to obtain a purified water-soluble vitamin E derivative mixture. In particular, the methods use the exemplary reaction mixtures described above. The methods to synthesize water-soluble vitamin E derivative mixtures that can be used in the soft-gel and pre-gel compositions described herein result in water-soluble vitamin E derivative mixtures, such as TPGS compositions, that are less turbid than known water-soluble vitamin E derivative mixtures, i.e., known compositions that contain more than 70% TPGS monomer and less than 12% TPGS dimer, when diluted in an aqueous medium, e.g., water.

The following methods are exemplary only and provide a platform from which adjustments can be made. It is understood that changes can be made to the steps of the method and to the reaction components while retaining some if not all of the desirable properties of the method. Further changes can be made by adding or altering steps or components of each step. For example, the order in which the steps are performed can be changed.

i. Preparation of a Crude Water-Soluble Vitamin E Derivative Mixture

An exemplary method of preparing a high dimer-containing mixture of TPGS is described. The method can be employed to produce high dimer-containing mixtures of any vitamin E derivative, including PEG derivatives of vitamin E. Exemplary is a method of preparing a crude water-soluble vitamin E derivative mixture, for example, a crude TPGS composition, by providing a reaction mixture containing vitamin E succinate, e.g. D-α-tocopheryl succinate, a polyethylene glycol, e.g., PEG 1000, a catalyst, e.g., p-toluenesulfonic acid, and a solvent, e.g., toluene, heating the reaction mixture to a temperature of at least or about at least 110° C. and maintaining the elevated temperature for a period of up to at or about 6.5 hours before cooling, for example, to room temperature, i.e., at or about 20° C., and washing the reaction mixture with an aqueous solution of a weak base, e.g., a 10% aqueous solution of sodium bicarbonate.

A crude water-soluble vitamin E derivative mixture is prepared by providing a reaction mixture containing vitamin E succinate, a polyethylene glycol and optionally, a catalyst, in a solvent and heating the reaction mixture from room temperature, i.e., at or about 20° C., to an elevated temperature, and maintaining the elevated temperature for a period of time until a crude water-soluble vitamin E derivative mixture, for example, a crude TPGS composition, is formed that contains the desired amounts of TPGS monomer and TPGS dimer. The elevated temperature can be any temperature in the range of from 30° C. to about 300° C., generally between 80° C. and 250° C., such as between 100° C. and 200° C. The elevated temperature can be, for example, the boiling point of the solvent in the reaction mixture. A typical heating schedule can be heating the reaction mixture to a temperature of at least or about at least 110° C. with stirring, and once achieved, the elevated temperature, e.g., at least or about at least 110° C., is maintained for a total time of up to at or about 6.5 hours with stirring. Other heating temperatures and times can be used depending on the substrates, solvent and formation of the desired crude water-soluble vitamin E derivative mixture. For example, the total time the elevated temperature is maintained can be at least at or about 1 hour, at least at or about 1.5 hours, at least at or about 2 hours, at least at or about 2.5 hours, at least at or about 3 hours, at least at or about 3.5 hours, at least at or about 4 hours, at least at or about 4.5 hours, at least at or about 5 hours, at least at or about 5.5 hours, at least at or about 6 hours, or at least at or about 6.5 hours, or longer, before cooling.

After the elevated temperature has been maintained for the desired amount of time, e.g., the amount of time required to produce the desired amounts of TPGS monomer and TPGS dimer, the reaction mixture can be cooled to a temperature lower than the elevated temperature. For example, the reaction mixture can be cooled to room temperature, i.e., at or about 20° C., after heating at an elevated temperature for the desired amount of time. The reaction mixture can be heated to at least or about at least 110° C. for a total time of about 6.5 hours before cooling, e.g., to room temperature (i.e., at or about 20° C.), depending on the substrates, solvent and formation of the crude water-soluble vitamin E derivative mixture, for example, a crude TPGS composition, resulting in the desired amounts of TPGS monomer and TPGS dimer. One of skill in the art can perform the methods and, if necessary, empirically determine the appropriate reaction duration to produce the desired ratio of dimer to monomer, based on the formation of the desired amounts of TPGS monomer and TPGS dimer.

In the exemplary method, the reaction mixture can be heated from room temperature (i.e., at or about 20° C.) to an elevated temperature of at least at or about 30° C., 40° C., 50° C., 60° C., 70° C., 80° C., 90° C., 100° C., 105° C., 110° C., 115° C., 120° C., 125° C., 130° C., 140° C., 150° C., 155° C., 160° C., 165° C., 170° C., 175° C., 180° C., 185° C., 190° C., 195° C., 200° C., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., or higher. The reaction mixture can be maintained at a temperature elevated from room temperature for at least at or about 1 hour, 1.5 hours, 2 hours, 2.5 hours, 3 hours, 3.5 hours, 4 hours, 4.5 hours, 5 hours, 5.5 hours, 6 hours, 6.5 hours, or longer before cooling. In an exemplary method, the reaction mixture can be maintained at an elevated temperature for up to at or about 6.5 hours before cooling, e.g., to room temperature, i.e., at or about 20° C. The particular conditions depend upon the particular vitamin E derivative and the amount of monomer and dimer desired.

The amount of time that the reaction mixture is maintained at the temperature elevated from room temperature, for example, between or about between 30° C. and 300° C., such as the boiling point of the solvent in the reaction mixture, can be determined by monitoring the progress of reaction during heating. For example, the reaction mixture can be monitored during heating to determine the amounts of TPGS monomer and TPGS dimer present in the reaction mixture. The heating can then be terminated when the desired amounts of TPGS monomer and TPGS dimer are formed. The monitoring can be done by any method of monitoring a reaction known to those of skill in the art, such as by chromatography, spectroscopy or spectrometry. For example, the reaction can be monitored by thin layer chromatography (TLC), high performance liquid chromatography (HPLC), infrared spectroscopy (IR), Fourier transform infrared spectroscopy (FTIR), mass spectrometry (MS), nuclear magnetic resonance (NMR) spectroscopy, or any combination thereof. In some embodiments of the methods, the reaction progress is monitored by TLC. In other embodiments, the reaction progress is monitored by HPLC. In yet other embodiments, the reaction progress is monitored by both TLC and HPLC. One of skill in the art, if necessary, can determine particular parameters empirically, such as appropriate reaction duration, based on monitoring the formation of the desired amounts of vitamin E derivative monomer and dimer, such as TPGS monomer and TPGS dimer.

The reaction mixture can be heated to an elevated temperature under an inert gas atmosphere, such as a nitrogen gas or argon gas atmosphere, or under air. The reaction mixture can be heated to an elevated temperature at atmospheric pressure or at an elevated pressure, i.e., a pressure higher than atmospheric pressure. The elevated pressure can be achieved, e.g., by performing the reaction in a closed vessel or in a vented vessel.

The progress of the reaction can be terminated after heating for the desired amount of time, for example, up to at or about 6.5 hours, by cooling the reaction mixture, for example, to room temperature, i.e., at or about 20° C. After cooling, such as cooling to room temperature, i.e., at or about 20° C., the reaction mixture can be washed with an aqueous solution. The aqueous solution can be an aqueous solution of base, such as a weak base, i.e., bases that do not fully ionize in an aqueous solution. Suitable weak bases include, for example, carbonates or bicarbonates, e.g., sodium carbonate, sodium bicarbonate, potassium carbonate and potassium bicarbonate; amines, ammonias or ammoniums, e.g., methyl amine, methyl ethyl amine, dimethyl amine, aniline, ammonia, trimethyl ammonia and ammonium hydroxide; and pyridine. For example, the aqueous solution of base can be an aqueous solution of sodium bicarbonate. Suitable aqueous solutions of the weak base include solutions that contain, e.g., 1% to 20% weak base, such as at least or about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, or more, weak base. For example, the aqueous solution can be an aqueous solution containing at or about 10% sodium bicarbonate. After the aqueous solution of a weak base has been added to the reaction mixture, the aqueous solution can be separated from the reaction mixture, such as by allowing the reaction mixture and aqueous solution of weak base to separate into layers, and removed. In some embodiments, the reaction mixture and aqueous solution of weak base can be stirred for a period of time before separating. For example, the reaction mixture and aqueous solution can be stirred for 1 minute, 2 minutes, 3 minutes, 4 minutes, 5 minutes, 6 minutes, 7 minutes, 8 minutes, 9 minutes, 10 minutes, 15 minutes, 20 minutes, or more, before allowing the reaction mixture and aqueous solution of weak base to separate into layers.

ii. Processing the Reaction Mixture to Obtain a Crude Water-Soluble Vitamin E Derivative Mixture

After preparing the reaction mixture, the reaction mixture can be further processed in order to obtain a crude water-soluble vitamin E mixture, for example, a crude TPGS composition that contains less TPGS monomer, i.e., less than 70 wt %, and more TPGS dimer, i.e., more than 12 wt %, than known water-soluble vitamin E derivative mixtures. The further processing can be performed to remove impurities from the reaction mixture before obtaining the crude water-soluble vitamin E derivative mixture. The further processing can be performed in order to isolate the crude water-soluble vitamin E derivative mixture from the reaction mixture. For example, the reaction mixture can be further processed by treating the reaction mixture with an adsorbent, such as activated charcoal (i.e., activated carbon). Activated charcoal can be used as a decolorizer and to remove impurities by chemical adsorption. Any activated charcoal known to those of skill in the art can be used to treat the reaction mixture. Such activated charcoal is available from commercial sources under such trade names as Calgon-Type CPG®, Type PCB®, Type SGL®, Type CAL®, and Type OL®.

Further processing of the reaction mixture, for example, treating the reaction mixture with activated charcoal, can take place for a period of time of from at or about 0.5 hours to at or about 5 hours, or longer if required. For example, treating the reaction mixture with activated charcoal can take place for at least or about at least 0.5 hours, 1 hour, 1.5 hours, 2 hours, 3 hours, 4 hours, 5 hours, or longer. The further processing, for example, treating the reaction mixture with activated charcoal, can be done at any temperature of from at or about room temperature, i.e., at or about 20° C., to a temperature elevated from room temperature. For example, the temperature of the process, e.g., activated charcoal treatment, can be at or about 20° C., 30° C., 40° C., 50° C., 55° C., 60° C., 70° C., 80° C., 90° C., or 100° C., or any temperature between 20° C. and 100° C., such as between or about between 55° C. and 60° C. The treatment temperatures and times can be varied depending on the reaction mixture, the solvent, and the impurities present in the reaction mixture. In an exemplary process, such as an activated charcoal treatment process, the reaction mixture can be treated, e.g., with activate charcoal, for at least or about at least 1 hour at a temperature of between or about between 55° C. and 60° C., before cooling.

The reaction mixture can be filtered and washed after cooling, such as cooling to room temperature, i.e., at or about 20° C., after further processing, such as after treating the reaction mixture with activated charcoal. The reaction mixture can be filtered and washed, for example, to remove the activated charcoal from the reaction mixture. For example, the reaction mixture can be filtered through a filter aid, such as diatomaceous earth. Suitable filter aids for use in the methods include, for example, those sold under the trademark Celite®, such as those sold under the trademark Hyflo®. After filtering through a filter aid, such as diatomaceous earth, the reaction mixture can be washed, for example, with the same solvent used in the reaction mixture. In an exemplary embodiment, after further processing, e.g., treatment with activated charcoal, and cooling, e.g., to room temperature, i.e., at or about 20° C., the reaction mixture is filtered through diatomaceous earth, e.g., Hyflo® filter aid, and washed with solvent, e.g., toluene.

The reaction mixture can be further processed in order to isolate the crude water-soluble vitamin E derivative mixture from the reaction mixture. For example, the reaction mixture can be further processed by removing the solvent from the reaction mixture, i.e., concentrating the reaction mixture, in order to obtain a crude water-soluble vitamin E derivative mixture. Any method of removing a solvent from a reaction mixture known to those of skill in the art can be used, including, for example, vacuum distillation, rotary evaporation and filtration. Removing the solvent from the reaction mixture can be done at any temperature, for example at room temperature, i.e., 20° C., or at a temperature elevated from room temperature. For example, the solvent can be removed at a temperature of at or about 20° C., 30° C., 40° C., 50° C., 55° C., 60° C., 70° C., 80° C., or 90° C., but below or about below 100° C., such as below or about below 60° C. In an exemplary embodiment, the solvent can be removed from the reaction mixture by distillation, e.g., vacuum distillation, at a temperature elevated from room temperature, i.e., at or about 20° C., but below or about below 60° C.

Further processing of the reaction mixture of the methods can include further processing by treating the reaction mixture to remove impurities from the reaction mixture, such as by treating the reaction mixture with activated charcoal. Further processing of the reaction mixture of the methods can include further processing by removing the solvent from the reaction mixture, such as by removing the solvent by vacuum distillation. The further processing can include treating the reaction mixture with activated charcoal or removing the solvent from the reaction mixture or both. In an exemplary method, the further processing of the reaction mixture includes removing the impurities from the reaction mixture, e.g., treating the reaction mixture with activated charcoal, and removing the solvent from the reaction mixture, e.g., removing the solvent by vacuum distillation, in order to obtain a crude water-soluble vitamin E derivative mixture, for example, a crude TPGS composition, containing less TPGS monomer, i.e., less than 70 wt %, and more TPGS dimer, i.e., more than 12 wt %, than in known TPGS compositions.

iii. Purification of the Crude Water-Soluble Vitamin E Derivative Mixture to Obtain a Purified High Dimer-Containing Water-Soluble Vitamin E Derivative Mixture

The crude water-soluble vitamin E derivative mixture obtained after further processing can be further purified in order to obtain a purified high dimer-containing water-soluble vitamin E derivative mixture. For example, the purified water-soluble vitamin E derivative mixture can be a PEG derivative of vitamin E, such as TPGS, PTS, PTD and other TPGS analogs and PEG derivatives of vitamin E, mixture. The mixture contains less TPGS monomer, i.e., less than 70 wt %, and more TPGS dimer, i.e., more than 12, 19, 24, 29 wt % dimer. The purification process removes impurities from the crude water-soluble vitamin E derivative mixture, such as impurities that were not removed by further processing of the reaction mixture. For example, the crude water-soluble vitamin E derivative mixture can be purified by performing one or more wash, i.e., extraction, steps. The wash can be performed using more than one solvent, such as more than one organic solvent, for example, two organic solvents that are not miscible with each other. For example, in the methods, the crude water-soluble vitamin E derivative mixture can be dissolved in a first solvent, for example, a polar solvent, such as an alcohol, and can be washed with a second solvent, for example, a non-polar solvent, such as a hydrocarbon solvent that is not miscible with the first solvent. The purification process, e.g., the wash, can be performed one time, two times, three times, four times, or more, depending on the desired purity level of the water-soluble vitamin E derivative mixture and the amount of impurities present. For example, the purification process, e.g., the wash, can be performed one or more times on the crude water-soluble vitamin E derivative mixture, e.g., after the crude water-soluble vitamin E derivative mixture is obtained after processing. In an exemplary method, the purification process can be performed three or more times on the crude water-soluble vitamin E derivative mixture after the further processing is complete.

The purification process, i.e., the wash, can be performed by dissolving the crude water-soluble vitamin E derivative mixture in a first solvent, for example, an organic solvent, such as a polar organic solvent. The polar organic solvent can be any solvent that can dissolve the crude water-soluble vitamin E derivative mixture, such as a polar protic solvent, for example, an alcohol, e.g., methanol, ethanol, propanol or butanol. In the methods, the amount of first solvent, e.g., polar organic solvent, used to dissolve the crude water-soluble vitamin E derivative mixture can be based on the ratio of the volume of the first solvent to the volume of the crude water-soluble vitamin E derivative mixture. The ratio of the volume of the first solvent to the volume of the crude water-soluble vitamin E derivative mixture can range from 0.1:1 to 10:1. In some embodiments, the ratio of the volume of the first solvent to the volume of the crude TPGS composition is or is about 0.1:1, 0.2:1, 0.25:1, 0.3:1, 0.4:1, 0.45:1, 0.5:1, 0.6:1, 0.7:1, 0.75:1, 0.8:1, 0.9:1, 1:1, 1.2:1, 1.25:1, 1.3:1, 1.4:1, 1.5:1, 1.6:1, 1.7:1, 1.75:1, 1.8:1, 1.9:1, 2:1, 2.5:1, 3:1, 3.5:1, 3.6:1, 4:1, 4.5:1, 5:1, 5.5:1, 6:1, 6.5:1, 7:1, 7.5:1, 8:1, 8.5:1, 9:1, 9.5:1, or 10:1 or more. For example, the ratio of the volume of the first solvent to the volume of the crude water-soluble vitamin E derivative mixture can be 2:1.

The wash can be performed using a second solvent, for example, an organic solvent, that is not miscible with the first solvent, i.e., the solvent used to dissolve the crude water-soluble vitamin E derivative mixture. The second solvent can be any solvent that is not miscible with the first solvent, for example, any solvent that is not miscible with a polar protic solvent such as an alcohol. Suitable organic solvents that can be used as a second solvent include non-polar organic solvents, such as hydrocarbons, e.g., alkanes and cycloalkanes, such as hexane and cyclohexane; halogenated hydrocarbons, e.g., chloroform and dichloromethane; ethers, e.g., diethyl ether; and aromatics, e.g., benzene and toluene. In the methods, the amount of second solvent, e.g., a non-polar organic solvent immiscible with the first solvent, used to wash the crude water-soluble vitamin E derivative mixture dissolved in the first solvent can be based on the ratio of the volume of the second solvent to the volume of the crude water-soluble vitamin E derivative mixture. The ratio of the volume of the second solvent to the volume of the crude water-soluble vitamin E derivative mixture can range from 0.1:1 to 10:1. In some embodiments, the ratio of the volume of second solvent to the volume of crude water-soluble vitamin E derivative mixture is or is about 0.1:1, 0.2:1, 0.25:1, 0.3:1, 0.4:1, 0.45:1, 0.5:1, 0.6:1, 0.7:1, 0.75:1, 0.8:1, 0.9:1, 1:1, 1.2:1, 1.25:1, 1.3:1, 1.4:1, 1.5:1, 1.6:1, 1.7:1, 1.75:1, 1.8:1, 1.9:1, 2:1, 2.5:1, 3:1, 3.5:1, 3.6:1, 4:1, 4.5:1, 5:1, 5.5:1, 6:1, 6.5:1, 7:1, 7.5:1, 8:1, 8.5:1, 9:1, 9.5:1, or 10:1 or more. For example, the ratio of the volume of the second solvent to the volume of the crude water-soluble vitamin E derivative mixture can be 3:1.

The purification process, for example, a wash with organic solvent, can be performed one or more times on the crude water-soluble vitamin E derivative mixture, for example, two times, three times, four times, or more. The wash can be performed while stirring. In an exemplary method, the crude water-soluble vitamin E derivative mixture can be dissolved in a first solvent, for example, a protic polar organic solvent, e.g., an alcohol, and washed three or more times with a second solvent, for example, a non-polar organic solvent not miscible in the first solvent, e.g., a hydrocarbon.

Exemplary is a method of purifying a crude water-soluble vitamin E derivative mixture by performing a purification process, such as a wash with an organic solvent, e.g., by dissolving the crude water-soluble vitamin E derivative mixture in methanol and washing with cyclohexane, and repeating the wash with the cyclohexane three or more times.

The crude water-soluble vitamin E derivative mixture can be further purified in order to obtain a purified water-soluble vitamin E derivative mixture, for example, a purified TPGS composition. The purified water-soluble vitamin E derivative mixture can be a purified TPGS composition that contains less TPGS monomer, i.e., less than 70 wt %, and more TPGS dimer, i.e., more than 12 wt %, than known TPGS compositions. The further purification can be performed to remove impurities from the crude water-soluble vitamin E derivative mixture. The further purification can be performed in order to isolate the purified water-soluble vitamin E derivative mixture from the first solvent. For example, the crude water-soluble vitamin E derivative mixture can be further purified by treating the crude water-soluble vitamin E derivative mixture with an adsorbent, such as activated charcoal (i.e., activated carbon). Activated charcoal can be used as a decolorizer and to remove impurities by chemical adsorption. Any activated charcoal known to those of skill in the art can be used to treat the crude water-soluble vitamin E derivative mixture. Such activated charcoal is available from commercial sources under such trade names as Calgon-Type CPG®, Type PCB®, Type SGL®, Type CAL®, and Type OL®.

Further purification of the crude water-soluble vitamin E derivative mixture, for example, treating the crude water-soluble vitamin E derivative mixture with activated charcoal, can take place for a period of time of from at or about 0.5 hours to at or about 5 hours, or longer if required. The crude water-soluble vitamin E derivative mixture to be treated can be dissolved in a solvent, for example, the first solvent used in the wash described above. Additional solvent can be added, for example, the same solvent used to dissolve the crude water-soluble vitamin E derivative mixture during the wash, e.g., a polar protic organic solvent. In the methods, the amount of additional solvent, e.g., polar protic organic solvent, added to the crude water-soluble vitamin E derivative mixture can be based on the ratio of the total volume of the solvent, e.g., the first solvent, such as a polar protic organic solvent, plus the additional solvent, to the volume of the crude water-soluble vitamin E derivative mixture. The ratio of the total volume of the first solvent plus the additional solvent to the volume of the crude TPGS composition can range from 0.1:1 to 10:1. In some embodiments, the ratio of the volume of total solvent to the volume of crude water-soluble vitamin E derivative mixture is or is about 0.1:1, 0.2:1, 0.25:1, 0.3:1, 0.4:1, 0.45:1, 0.5:1, 0.6:1, 0.7:1, 0.75:1, 0.8:1, 0.9:1, 1:1, 1.2:1, 1.25:1, 1.3:1, 1.4:1, 1.5:1, 1.6:1, 1.7:1, 1.75:1, 1.8:1, 1.9:1, 2:1, 2.5:1, 3:1, 3.5:1, 3.6:1, 4:1, 4.5:1, 5:1, 5.5:1, 6:1, 6.5:1, 7:1, 7.5:1, 8:1, 8.5:1, 9:1, 9.5:1, or 10:1 or more. For example, the ratio of the total volume of the first solvent plus additional solvent to the volume of the crude water-soluble vitamin E derivative mixture can be 5:1.

Further purification, such as treating the reaction mixture with, for example, activated charcoal, can take place for at least or about at least 0.5 hours, 1 hour, 1.5 hours, 2 hours, 3 hours, 4 hours, 5 hours, or longer. The further purification, for example, treating the reaction mixture with activated charcoal, can be done at any temperature of from at or about room temperature, i.e., at or about 20° C., to a temperature elevated from room temperature. For example, the temperature of the purification process, e.g., activated charcoal treatment, can be at or about 20° C., 30° C., 40° C., 50° C., 55° C., 60° C., 70° C., 80° C., 90° C., or 100° C., or any temperature between 20° C. and 100° C., such as between or about between 55° C. and 60° C. The treatment temperatures and times can be varied depending on the nature of the crude water-soluble vitamin E derivative mixture, the solvent, and the impurities present in the crude water-soluble vitamin E derivative mixture. In an exemplary purification process, such as an activated charcoal treatment process, the crude water-soluble vitamin E derivative mixture can be treated, e.g., with activate charcoal, for at least or about at least 1 hour at a temperature of between or about between 55° C. and 60° C., before cooling.

The crude water-soluble vitamin E derivative mixture can be filtered and washed after cooling, such as cooling to room temperature, i.e., at or about 20° C., after further purification, such as after treating the crude water-soluble vitamin E derivative mixture with activated charcoal. The crude water-soluble vitamin E derivative mixture, for example, the crude water-soluble vitamin E derivative mixture dissolved in a solvent, can be filtered and washed, for example, to remove the activated charcoal from the crude water-soluble vitamin E derivative mixture. For example, the crude water-soluble vitamin E derivative mixture, for example, the crude water-soluble vitamin E derivative mixture dissolved in a solvent, can be filtered through a filter aid, such as diatomaceous earth. Suitable filter aids for use in the methods include, for example, those sold under the trademarks Celite® and Hyflo®. After filtering through a filter aid, such as diatomaceous earth, the crude TPGS composition can be washed, for example, with the same solvent used to dissolve the crude water-soluble vitamin E derivative mixture, e.g., the first solvent. In an exemplary embodiment, after further purification, e.g., treatment with activated charcoal, and cooling, e.g., to room temperature, i.e., at or about 20° C., the crude water-soluble vitamin E derivative mixture is filtered through diatomaceous earth, e.g., Hyflo® filter aid and washed with solvent, e.g., methanol.

The crude water-soluble vitamin E derivative mixture can be further purified in order to isolate the purified water-soluble vitamin E derivative mixture from the solvent, e.g., the first solvent. For example, the crude water-soluble vitamin E derivative mixture can be further purified by removing the solvent from the water-soluble vitamin E derivative mixture dissolved in solvent, i.e., concentrating the crude water-soluble vitamin E derivative mixture, in order to obtain a purified water-soluble vitamin E derivative mixture. Any method of removing a solvent from a composition known to those of skill in the art can be used, including, for example, vacuum distillation, rotary evaporation and filtration. Removing the solvent from the water-soluble vitamin E derivative mixture can be done at any temperature, for example at room temperature, i.e., 20° C., or at a temperature elevated from room temperature. For example, the solvent can be removed at a temperature of at or about 20° C., 30° C., 40° C., 50° C., 55° C., 60° C., 70° C., 80° C., or 90° C., but below or about below 100° C., such as below or about below 60° C. In an exemplary embodiment, the solvent can be removed from the crude water-soluble vitamin E derivative mixture by distillation, e.g., vacuum distillation, at a temperature elevated from room temperature, i.e., at or about 20° C., but below or about below 60° C. After removing the solvent, the purified water-soluble vitamin E derivative mixture can be dried by any method of drying known to those of skill in the art. Suitable methods of drying include drying under an inert gas, for example, nitrogen or argon, or drying under vacuum, or any combination thereof.

Further purification of the crude water-soluble vitamin E derivative mixture produced by the exemplified method can include further purification by treating the crude water-soluble vitamin E derivative mixture to remove impurities from the reaction mixture, such as by treating the crude water-soluble vitamin E derivative mixture with activated charcoal. Further purification of the crude water-soluble vitamin E derivative mixture produced by the exemplified method can include further purification by removing the solvent from the crude water-soluble vitamin E derivative mixture, for example, a crude water-soluble vitamin E derivative mixture dissolved in a solvent, such as by removing the solvent by vacuum distillation. The further purification can include treating the crude water-soluble vitamin E derivative mixture with activated charcoal or removing the solvent from the crude water-soluble vitamin E derivative mixture or both. In an exemplary method, the further purification of the crude water-soluble vitamin E derivative mixture includes removing the impurities from the crude water-soluble vitamin E derivative mixture, e.g., treating the crude water-soluble vitamin E derivative mixture with activated charcoal, and removing the solvent from the crude water-soluble vitamin E derivative mixture, e.g., removing the solvent by vacuum distillation, in order to obtain a purified water-soluble vitamin E derivative mixture, for example, a purified TPGS composition. The purified TPGS composition can contain less TPGS monomer, i.e., less than 70 wt %, and more TPGS dimer, i.e., more than 12 wt %, than in known TPGS compositions.

The exemplified methods yield a purified water-soluble vitamin E derivative mixture, such as a purified TPGS composition, with the desired amount of dimer (greater than 12%) that can be used in any application where water-soluble vitamin E derivative mixtures are used, such as in food, beverage, pharmaceutical or nutraceutical products for human consumption, and particularly to prepare soft-gels and pre-gels, such as the soft-gels and pre-gels described herein, that contain the water-soluble vitamin E derivative composition and a non-polar ingredient(s) and other optional ingredients. For example, a purified water-soluble vitamin E derivative mixture, such as a purified TPGS composition, for example, a TPGS composition that contains less TPGS monomer, i.e., less than 70 wt %, and more TPGS dimer, i.e., more than 12 wt %, than in known TPGS compositions, that can be used in products for human consumption, for example, food and beverage products, particularly aqueous food and beverage products, and any other application in which a water-soluble vitamin E derivative mixture can be added, is produced. Exemplary purified water-soluble vitamin E derivative mixtures (compositions) that can be prepared following the exemplified methods are those that contain less than 70 wt % monomer and more than 12 wt % dimer, such as such as compositions containing between or about between 25 wt % and 69 wt % monomer and between or about between 13 wt % and 95 wt % dimer, such as compositions containing between or about between 40 wt % and 60 wt % monomer and between or about between 25 wt % to 60 wt % dimer. For example, the methods can be followed to obtain water-soluble vitamin E derivative mixtures (compositions) that contain between or about between 25 wt % and 69 wt % monomer, for example, at or about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69 wt % monomer and between or about between 13 wt % and 95 wt % dimer, for example, at or about 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94 or 95 wt % dimer.

These methods are described with reference to TPGS and can be adapted to produce any higher dimer-containing water-soluble vitamin E derivative composition. Other methods to produce compositions with the desired dimer or dimer and monomer concentrations can be employed, including purifying dimer from standard preparations and adding the dimer back to a standard preparation to increase its concentration. The resulting compositions can be employed in the soft-gels and pre-gels described herein.

4) Surfactant Properties

Properties of the provided pre-gel concentrates and soft gel compositions, for example, desirable properties related to bioavailability of the non-polar compounds, are influenced by the choice of surfactant and the relative amount (concentration) of surfactant. For example, the concentration of the surfactant compared with the concentration of other ingredients, particularly compared with the concentration of non-aqueous solvent and non-polar ingredients, can affect various desirable properties, for example, the ability of the non-polar ingredients to remain in solution and not precipitate, or the extent of bioavailability of the non-polar compounds after, for example, human consumption.

Exemplary of a property of the surfactant that contributes to the desirable properties of the pre-gel concentrates and soft gel compositions is the HLB (hydrophobic-lipophilic balance) of the surfactant. For example, the HLB of the surfactant can affect taste, smell, crystal formation, bioavailability, and other properties of the provided concentrates and compositions. Generally, HLB is a value, derived from a semi-empirical formula, used to index surfactants according to their relative hydrophobicity/hydrophilicity. An HLB value is a numerical representation of the relative representation of hydrophilic groups and hydrophobic groups in a surfactant or mixture of surfactants. The weight percent of these respective groups indicates properties of the molecular structure. See, for example, Griffin, W. C. J. Soc. Cos. Chem. 1:311 (1949).

Surfactant HLB values range from 1-45, while the range for non-ionic surfactants typically is from 1-20. The more lipophilic a surfactant is, the lower its HLB value. Conversely, the more hydrophilic a surfactant is, the higher its HLB value. Lipophilic surfactants have greater solubility in oil and lipophilic substances, while hydrophilic surfactants dissolve more easily in aqueous liquids. In general, surfactants with HLB values greater than 10 or greater than about 10 are called “hydrophilic surfactants,” while surfactants having HLB values less than 10 or less than about 10 are referred to as “hydrophobic surfactants.” HLB values are known for a number of surfactants.

Typically, surfactants used in the provided methods and compositions are surfactants having an HLB value of between 12 or about 12 and 20 or about 20, for example, 12, 13, 14, 15, 16, 17, 18, 19, 20, or about 12, about 13, about 14, about 15, about 16, about 17, about 18, about 19 or about 20. Exemplary of suitable surfactants include, but are not limited to, vitamin E-derived surfactants, such as tocopherol and/or tocotrienol-derived surfactants, in which the vitamin E moiety represents the hydrophobic region of the surfactant, and is attached, via a linker, to another moiety, such as a polyethylene glycol (PEG) moiety, that provides the hydrophilic portion of the surfactant. Vitamin-E derived surfactants include, but are not limited to, tocopherol derived surfactants, including polyalkylene glycol derivatives of tocopherol, typically polyethylene glycol (PEG) derivatives of tocopherol, such as tocopherol polyethylene glycol succinate (TPGS), TPGS analogs, TPGS homologs and TPGS derivatives. Alternatively, the surfactants can be other PEG derivatives having similar properties, for example, PEG derivatives of sterols, e.g., a cholesterol or a sitosterol (including, for example, any of the PEG derivatives disclosed in U.S. Pat. No. 6,632,443) or PEG derivatives of other fat-soluble vitamins, for example, some forms of vitamin A (e.g., retinol) or vitamin D (e.g., vitamins D1-D5). Typically, the surfactant is a natural surfactant, for example, a surfactant that is GRAS (generally recognized as safe) by the FDA and/or Kosher certified, for example, TPGS.

c. Non-Aqueous Solvents

The pre-gel concentrates provided herein contain at least one non-aqueous solvent, for example, any pharmaceutically acceptable solvent that is non-aqueous that can dissolve the non-polar compounds and ingredients provided herein. The non-aqueous solvent, such as, but not limited to, benzyl alcohol, is included in the composition in a high amount, e.g., between 5% or about 5% and 20% or about 20%, such as, 5% to 15%, 10% to 15%, 5% to 10%, for example, at least 5%, 6%, 7%, 8%, 9%, 10%, 11%, 12%, 13%, 14%, 15%, 16%, 17%, 18%, 19%, 20%, up to about or 20% or 25%. The non-aqueous solvents dissolve the non-polar ingredients. The non-aqueous solvent is one that dissolves the non-polar ingredients and is other than or does not contain the non-polar ingredient. The non-aqueous solvent prevents the non-polar ingredients from precipitating out of the concentrate. The use of these higher amounts of non-aqueous solvent has been found herein to provide advantageous properties, for example, in the pre-gel concentrates and the soft gel compositions provided herein when the soft gel compositions contain a pre-gel concentrate containing non-polar ingredients and a surfactant along with high amounts of non-aqueous solvent. For example, the concentrates and soft gels do not contain precipitated non-polar ingredients and provide enhanced bioavailability of the non-polar compounds in the soft gel compositions, for example, after consumption.

The one or more non-aqueous solvents included in the pre-gel concentrates are typically insoluble or only partial soluble in water. For example, the non-aqueous solvents can include, but are not limited to, alkanes, e.g., hydrocarbons and cyclic hydrocarbons, e.g., d-limonene; aromatic compounds, such as aromatic alcohols, ethers and esters, e.g., benzyl alcohol and benzyl benzoate; haloalkanes, ethers, esters and ketones; organic solvents of natural origin, such as natural hydrocarbons, for example, cyclic hydrocarbons, e.g., terpenes, for example, d-limonene, alpha-pinene, beta-pinene, myrcene, linalol, citronellol, geraniol, menthol, citral, citronellal, or oxidized organic derivatives, in particular ethers, aldehydes, alcohols and esters; lactams, e.g., N-methyl pyrrolidone (NMP); alkylene glycols, e.g., propylene glycol and polyethylene glycol (e.g., PEG300, PEG400, etc.); dimethyl sulfoxide; dimethyl acetamide; 2-pyrrolidone; C2-C6 alkanols; 2-ethoxyethanol; alkyl esters such as 2-ethoxyethyl acetate, methyl acetate, ethyl acetate, ethylene glycol diethyl ether, or ethylene glycol dimethyl ether; (S)-(−)-ethyl lactate; acetone; glycerol; alkyl ketones such as methylethyl ketone or dimethyl sulfone; tetrahydrofuran; cyclic alkyl amides such as caprolactam; decylmethylsulfoxide; oleic acid; aromatic amines such as N,N-diethyl-m-toluamide; 1-dodecylazacycloheptan-2-one; natural products, such as oils, including olive oils and fatty acids, which can be saturated or non-saturated; and acyl glycerols. The pre-gel concentrates provided herein can contain one non-aqueous solvent or more than one non-aqueous solvent, for example, two, three, or more non-aqueous solvents.

Typically, the non-aqueous solvent is an alcohol, an alcohol derivative, and/or a hydrocarbon that has little or no solubility in water. Exemplary of non-aqueous solvents that can be included in the pre-gel concentrates are alcohols, for example, aromatic alcohols, e.g., benzyl alcohol, such as the benzyl alcohol sold by Sigma Aldrich (St. Louis, Mo.); alcohol derivatives, for example, ester derivatives of alcohols, such as aromatic esters, e.g., benzyl benzoate; hydrocarbons, for example, cyclic hydrocarbons, e.g., d-limonene, such as d-limonene that is 99% GRAS-certified, sold by Florida Chemical, Winter Haven, Fla.; and any other non-aqueous solvent that is insoluble or has only partial solubility in water.

The pre-gel compositions provided herein include a non-aqueous solvent that also can act as a preservative. Exemplary of such solvents is benzyl alcohol, in an amount that is significantly higher than the amount at which it is effective as an anti-microbial preservative. Hence, the compositions provided herein generally do not require additional preservatives.

d. Other Ingredients

1) Co-Surfactants

The concentrates can further contain one or more co-surfactants other than the primary co-surfactant, for example, the polyalkylene glycol derivative of vitamin E, e.g., TPGS. For example, a co-surfactant can be included to improve the stability of the composition, for example, by preventing or slowing oxidation of the non-polar ingredient. Exemplary of a co-surfactant that can be used in the provided concentrates is a phospholipid, for example, phosphatidylcholine. Other exemplary co-surfactants include non-ionic surfactants, such as sugar-derived surfactants, including fatty acid esters of sugars and sugar derivatives, and PEG-derived surfactants, such as PEG derivatives of sterols, PEG derivatives of fat-soluble vitamins and PEG-sorbitan fatty acid esters.

When present, the amount of the co-surfactant typically is present in a concentration less than or less than about 10%, typically less than or less than about 5%, for example, the total amount of co-surfactant as a percentage (%), by weight, of the liquid concentrate (wt %) can be, e.g., less than or less than about 10%, such as less than or about 5%, 4.5%, 4%, 3.5%, 3.15%, 3%, 2.5%, 2%, 1.75%, 1.5%, 1.25%, 1%, 0.75%, 0.5%, 0.25%, 0.15% or less, by weight, of the concentrate.

i. Phospholipids

Exemplary of the co-surfactants that can be used in the provided compositions are phospholipids. Phospholipids are amphipathic lipid-like molecules, typically containing a hydrophobic portion at one end of the molecule and a hydrophilic portion at the other end of the molecule. A number of phospholipids can be used as ingredients in the provided compositions, for example, lecithin, including phosphatidylcholine (PC; lecithin), phosphatidylethanolamine (PE), distearoylphosphatidylcholine (DSPC), phosphatidylserine (PS), phosphatidylglycerol (PG), phosphatidic acid (PA), phosphatidylinositol (PI), sphingomyelin (SPM) or a combination thereof. Exemplary of the phospholipids that can be used as co-surfactants in the provided compositions are the phospholipids sold by Lipoid, LLC (Newark, N.J.), for example, Purified Egg Lecithins, Purified Soybean Lecithins, Hydrogenated Egg and Soybean Lecithins, Egg Phospholipids, Soybean Phospholipids, Hydrogenated Egg and Soybean Phospholipids, Synthetic Phospholipids, PEG-ylated Phospholipids and phospholipid blends. Exemplary of a phosphatidylcholine that can be used as a co-surfactant in the provided compositions is the phosphatidylcholine composition sold by Lipoid, LLC, under the name Lipoid S100, which is derived from soy extract and contains greater than or greater than about 95% phosphatidylcholine.

ii. Sugar-Derived Surfactants

Exemplary sugar-derived surfactants include, but are not limited to, sugar fatty acid esters including fatty acid esters of sucrose, glucose, maltose and other sugars, esterified to fatty acids of varying lengths (e.g., containing a varying numbers of carbons). The fatty acids typically have carbon chains between 8 and 28 carbons in length, and typically between 8 and 20, or between 8 and 18 or between 12 and 18, such as, but not limited to, stearic acid (18 carbons), oleic acid (18 carbons), palmitic acid (16 carbons), myristic acid (14 carbons) and lauric acid (12 carbons). Typically, the sugar ester surfactants are sucrose ester surfactants, typically sucrose fatty acid ester surfactants.

Sucrose fatty acid ester (SFAE) surfactants contain one or more sucrose fatty acid esters, which are non-ionic surfactants that contain sucrose in the hydrophilic portions and fatty acids in the hydrophobic portions. The sucrose fatty acid esters can be made by well-known methods (see, for example, U.S. Pat. Nos. 3,480,616; 3,644,333; 3,714,144; 4,710,567; 4,898,935; 4,996,309; 4,995,911; 5,011,922 and 5,017,697 and International Patent Pub. No. WO 2007/082149), typically in an esterification reaction as described in U.S. Pub. No. 2012-0016026.

Because sucrose contains eight hydroxy (—OH) groups, the esterification reaction can join the sucrose molecule to one fatty acid molecule, or can join it to a plurality of fatty acid molecules, producing different degrees of esterification, e.g., mono-, di-, tri- and poly- (up to octa-) fatty acid esters, but primarily mono-, di- and/or tri-esters. The degree of esterification can depend on conditions of esterification. The esterification reaction can be carried out with a single type of fatty acid, or a plurality of fatty acids, such as fatty acids with varying carbon chain lengths, branched and linear fatty acids, and/or saturated or unsaturated fatty acids. The esterification reaction with a single fatty acid can produce a single ester, and typically forms more than one ester, such as mono- di-, tri- and/or poly-esters, formed from one reaction. The relative amounts of mono- di- tri- and/or poly-esters can depend on reaction conditions.

The fatty acid in the sucrose fatty acid ester can be any fatty acid, and can contain between 4 and 28 carbon atoms, typically between 8 and 28 carbon atoms, and typically between 8 and 25 carbon atoms, such as between 8 and 18 carbon atoms, such as 8, 9, 10, 11, 12, 13, 14, 15, 16, 17 and 18 carbon atoms. The fatty acid can be synthetic or naturally occurring, and include linear and branched fatty acids. The fatty acids include, but are not limited to, myristic acid, palmitic acid, stearic acid, oleic acid, caproic acid, capric (or decanoic) acid, lauric acid, caprylic acid and pelargonic (or nonanoic) acid.

Thus, sucrose fatty acid ester surfactants include sucrose monoesters, diesters, triesters and polyesters, and mixtures thereof, and typically contain sucrose monoesters. The sucrose fatty acid ester surfactants include single fatty acid esters and also include homogeneous mixtures of sucrose esters, containing members with different lengths of fatty acid carbon chain and/or members with different degrees of esterification. For example, the sucrose fatty acid ester surfactants include mixtures of monoesters, diesters, triesters, and/or polyesters. The sugar ester surfactants further include sucrose fatty acid ester analogs and homologs and mixtures thereof.

In general, sucrose fatty acid esters, including mixtures of sucrose fatty acid esters, can have varying HLB values, such as HLB values ranging from at or about 1 to at or about 20. The HLB value of the sucrose fatty acid ester generally depends on the degree of esterification (e.g., the average degree of esterification in a mixture of different esters). Typically, the lower the degree of esterification (e.g., average degree), the higher the HLB value of the sucrose fatty acid ester or mixture thereof. Exemplary sucrose esters include sucrose distearate (HLB=3), sucrose distearate/monostearate (HLB 12), sucrose dipalmitate (HLB=7.4), sucrose monostearate (HLB=15), sucrose monopalmitate (HLB>10), sucrose monolaurate (HLB 15). Typically, the sucrose fatty acid ester surfactants in the provided concentrates have an HLB value of between at or about 13 and at or about 20, such as at or about 13, 14, 15, 16, 17, 18, 19, or 20, and typically between at or about 13 and at or about 18, such as, but not limited to, HLB values of at or about 15, 16 and 17, such as, for example, sucrose ester surfactants including sucrose monopalmitate, sucrose monolaurate and sucrose monostearate.

The sugar ester surfactants include sucrose ester blends, for example, sucrose ester mixtures containing a specified amount (e.g., percent, by weight) of sucrose monoesters. Exemplary surfactants include sucrose ester mixtures having at least at or about 50%, by weight (w/w), monoester, such as at least or about at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%, by weight (w/w), sucrose monoesters, and typically at least at or about 60%, by weight, or at least at or about 70%, by weight (w/w), monoesters.

The sucrose fatty acid ester surfactants include sucrose fatty acid monoesters, such as sucrose monocaprylate, sucrose monodecanoate, sucrose monolaurate, sucrose monomyristate, sucrose monopalmitate, sucrose monostearate, sucrose monopelargonate, sucrose monoundecanoate, sucrose monotridecanoate, sucrose monopentadecanoate and sucrose monoheptadecanoate. The sucrose fatty acid esters further include mixtures containing varying percentages of monoesters, diesters, triesters and polyesters, such as, but not limited to, a mixture having at or about 72% monoesters, 23% diesters, 5% triesters and 0 polyesters; a mixture having at or about 61% monoesters, 30% diesters, 7% triesters, and 2% polyesters; and a mixture having at or about 52% monoesters, 36% diesters, 10% triesters and 2% polyesters.

The sucrose fatty acid ester surfactants include sucrose fatty acid esters sold under the trade name DK Ester®, produced by Dai-Ichi Kogyo Seiyaku Co., Ltd of Japan (which, in some examples, can be produced according to the methods described in U.S. Pat. Nos. 4,898,935; 4,996,309; 4,995,911; 5,011,922 and 5,017,697), and distributed through Montello Inc., Tulsa, Okla., such as the F-160 and F-140 grade esters sold under the trade name DK Ester®, and sucrose esters sold under the trade name SURFHOPE® SE PHARMA, by Mitsubishi-Kagaku Foods Corporation, distributed by Mitsubishi Chemical Performance Polymers, Inc. These sucrose fatty acid esters are mixtures of esters with different degrees of esterification. The sucrose fatty acid esters further include Ryoto sugar esters, which are food-grade esters sold by Mitsubishi-Kagaku Foods Corporation, distributed by Mitsubishi Chemical Performance Polymers, Inc. Other exemplary sucrose fatty acid ester surfactants are described in Youan et al. (2003) AAPS PharmaSci 5(2):Article 22 (1-9) and in Okamoto et al. (2005) Biol. Pharm. Bull. 28(9):1689-1694.

iii. PEG-Derived Surfactants

Exemplary PEG-derived surfactants include, but are not limited to, PEG derivatives of sterols, e.g., a cholesterol or a sitosterol (including, for example, any of the PEG derivatives disclosed in U.S. Pat. No. 6,632,443); PEG derivatives of fat-soluble vitamins, for example, some forms of vitamin A (e.g., retinol) or vitamin D (e.g., vitamin D1-D5); and PEG-sorbitan fatty acid esters, such as polysorbates, including polyoxyethylene (20) sorbitan monooleate (also called polysorbate 80) and analogs (e.g., homologs) of polysorbate 80, such as, for example, polysorbate 20 (polyoxyethylene (20) sorbitan monolaurate), polysorbate 40 (polyoxyethylene (20) sorbitan monopalmitate) and polysorbate 60 (polyoxyethylene (20) sorbitan monostearate); and stearic acid derivatives, including, for example, polyethylene glycol 400 distearate (PEG 400 DS), such as the PEG 400 DS sold by Stepan Lipid Nutrition (Maywood, N.J.).

2) Flavors

The compositions provided herein can further contain one or more flavors or flavoring agents, for example, any compound that can add flavor to the concentrate and/or to the soft gel composition containing the concentrate. Several flavors are well known. Any flavor can be added to the concentrates, for example, any flavor sold by Mission Flavors (Foothill Ranch, Calif.). Exemplary of flavors that can be used are fruit flavors, such as guava, kiwi, peach, mango, papaya, pineapple, banana, strawberry, raspberry, blueberry, orange, grapefruit, tangerine, lemon, lime and lemon-lime; cola flavors, tea flavors, coffee flavors, chocolate flavors, dairy flavors, root beer and birch beer flavors, methyl salicylate (wintergreen oil, sweet birch oil), citrus oils and other flavors. Typically, the flavors are safe and/or desirable for human consumption, for example, GRAS or Kosher-certified flavors. Exemplary flavoring agents that can be used in the concentrates and soft gel compositions provided herein are lemon oil, for example lemon oil sold by Mission Flavors (Foothill Ranch, Calif.), and d-limonene, for example, 99% GRAS certified d-Limonene, sold by Florida Chemical (Winter Haven, Fla.).

2. Formulating the Non-Aqueous Pre-Gel Compositions Containing Non-Polar Ingredients

The non-aqueous pre-gel compositions, i.e., concentrates, contain one or more non-polar ingredients; at least one surfactant, for example, a polyethylene glycol derivative of vitamin E, e.g., TPGS; and one or more non-aqueous solvents, for example, solvents that have little or no solubility in water, for example, an alcohol, ester, or hydrocarbon, or mixtures thereof.

As a first step in formulating the provided pre-gel concentrates, one or more initial pre-gel concentrates are made and evaluated for desired properties. For this step, ingredients are selected, for example, from one or more of the lists of ingredients provided above. A starting concentration (weight percentage) of each selected ingredient is selected from within an appropriate concentration range for that ingredient or category of ingredient. For example, a starting surfactant concentration, such as a polyethylene glycol derivate of vitamin E, e.g., TPGS, is selected from within an appropriate surfactant concentration range. In some cases, the initial pre-gel concentrate is formulated based on the ingredients, and concentrations thereof, of an existing pre-gel concentrate, having one or more desired properties.

The initial pre-gel concentrate(s) is then made, using the methods for making pre-gel concentrates described below, adding each ingredient at its starting concentration at the appropriate step. In one example, more than one initial pre-gel concentrate is made. For example, multiple initial pre-gel concentrates, each having a different concentration of one or more ingredients, can be made and compared. For example, multiple initial pre-gel concentrates can be made in order to test various representative concentrations within an appropriate concentration range for one or more particular ingredient.

Each of the provided non-aqueous pre-gel concentrates contains at least one non-polar compound, typically more than one non-polar compound, for example, non-polar ingredients that contain one or more non-polar compounds. Any non-polar ingredient that contains one or more non-polar compounds can be formulated with the provided methods and pre-gel concentrates. Several exemplary non-polar compounds and non-polar ingredients that can be incorporated into the provided concentrates are described herein above. Typically, the non-polar ingredient is or contains a non-polar compound, for example, an oil-based ingredient, for example, a polyunsaturated fatty acid (PUFA), a coenzyme Q, or a vitamin.

The non-aqueous pre-gel concentrates provided herein contain high amounts of non-polar ingredients, for example, between or between about 30 wt % or 35 wt % or 40 wt % and 90 wt % non-polar ingredient, such as between or between about 40% and 45%, 40% and 50%, 40% and 55%, 40% and 60%, 40% and 65%, 40% and 70%, 40% and 75%, 40% and 80%, 40% and 85%, 40% and 90%, 45% and 50%, 45% and 55%, 45% and 60%, 45% and 65%, 45% and 70%, 45% and 75%, 45% and 80%, 45% and 85%, 45% and 90%, 50% and 55%, 50% and 60%, 50% and 65%, 50% and 70%, 50% and 75%, 50% and 80%, 50% and 85%, 50% and 90%, 55% and 60%, 55% and 65%, 55% and 70%, 55% and 75%, 55% and 80%, 55% and 85%, 55% and 90%, 60% and 65%, 60% and 70%, 60% and 75%, 60% and 80%, 60% and 85%, 60% and 90%, 65% and 70%, 65% and 75%, 65% and 80%, 65% and 85%, 65% and 90%, 70% and 75%, 70% and 80%, 70% and 85%, 70% and 90%, 75% and 80%, 75% and 85%, 75% and 90%, 80% and 85%, 80% and 90%, and 85% and 90%, by weight of the pre-gel concentrate. The pre-gel concentrates that contain high amounts of non-polar ingredients and high amounts of non-aqueous solvent exhibit desirable properties, for example, the non-polar ingredients remain in solution in the non-aqueous solvent and do not precipitate out.

In addition to the non-polar ingredients, the non-aqueous pre-gel concentrates contain at least one surfactant, for example, a polyethylene glycol derivate of vitamin E. Typically, the surfactant has an HLB value between 12 or about 12 and 20 or about 20, for example, 12, 13, 14, 15, 16, 17, 18, 19, 20, about 12, about 13, about 14, about 15, about 16, about 17, about 18, about 19 or about 20. Exemplary of surfactants are those described above, including polyethylene glycol derivatives of vitamin E described herein, such as tocopherol polyethylene glycol succinate (TPGS), such as the TPGS, TPGS analogs, TPGS homologs and TPGS derivatives described herein, and other surfactants having similar properties to TPGS, for example, other surfactants having HLB values between 12 or about 12 and 20 or about 20. Typically, the surfactant is a natural surfactant, for example, a surfactant that is GRAS (generally recognized as safe)-certified by the FDA and/or Kosher certified, for example, TPGS.

In the non-aqueous pre-gel concentrates provided herein, the concentration of the surfactant, for example, the polyethylene glycol derivative of vitamin E, e.g., TPGS, is greater than 5% or about 5%, typically greater than 10% or about 10%, typically greater than 20% or about 20%, for example, greater than 25% or about 25%, for example, a concentration within the concentration range of between 5% or about 5% and 40% or about 40%, between 10% or about 10% and 40% or about 40%, typically between 20% or about 20% and 40% or about 40%, for example, between 25% or about 25% and 40% or about 40%, for example, at least, 20, 21, 22, 23, 24, 25, 26, 27, 28, 28.25, 28.4, 29, 30, 31, 32, 33, 34, 34.6, 35, 36, 37, 38, 39, or 40%, by weight, of the pre-gel concentrate.

The pre-gel concentrates contain one or more non-aqueous solvents, such as solvents that are insoluble or only partial soluble in water. Typically, the non-aqueous solvent is an alcohol, a hydrocarbon, and/or an ester that has little or no solubility in water. Exemplary of non-aqueous solvents that can be included in the pre-gel concentrates are aromatic alcohols, including, but are not limited to, benzyl alcohol, cyclic hydrocarbons, e.g., d-limonene, aromatic esters, e.g., benzyl benzoate, and any non-aqueous solvent that is insoluble or has only partial solubility in water. Typically, an aqueous solvent, e.g., water, is not added as an ingredient to the pre-gel concentrate. In the pre-gel concentrates provided herein, the concentration of the non-aqueous solvent, or the total of all of the one or more non-aqueous solvents, is chosen from within a concentration range of between 5% or about 5% and 20% or about 20%, for example, at least 5, 6, 7, 7.5, 8, 9, 10, 10.6, 11, 12, 13, 14, 15, 15.4, 16, 17, 18, 19, or 20%, by weight, of the pre-gel concentrate.

A number of parameters of the pre-gel concentrates, including ingredients, their relative concentrations, and methods for making the pre-gel concentrates, affect the desirable properties of the concentrate, for example, the ability of the non-polar ingredients to remain in solution and not precipitate when a high concentration of non-polar ingredient is present. By extension, these parameters of the pre-gel concentrates also affect the desirable properties of the soft gel compositions, for example, the bioavailability of the non-polar compounds in the soft gel composition after consumption, for example, human consumption. In particular, the nature of the surfactant, particularly the HLB of the surfactant, and the non-aqueous solvent, and the relative concentrations of the surfactant, non-aqueous solvent, and non-polar ingredients in the pre-gel concentrates, contribute to the desirable properties of the pre-gel concentrates and thus, the soft gel compositions.

Accordingly, properties of the ingredients and their relative concentrations in the pre-gel concentrates are important for the ability of the pre-gel concentrates to yield desirable soft gel compositions. Selecting the appropriate ingredients, and relative concentrations thereof, within the ranges described herein, produce pre-gel concentrates that can be introduced into gel capsules to yield soft gel compositions having desirable properties.

3. Soft Gel Compositions Containing Non-Polar Ingredients

a. Capsules

The pre-gel concentrates are introduced into soft gel capsules to produce the soft gels provided. The soft gel compositions contain a non-aqueous pre-gel concentrate within a shell or coating, typically, a gel or similar capsule. The non-aqueous pre-gel concentrates described herein that contain non-polar ingredients, a surfactant, and non-aqueous solvent, are encapsulated in a coating or shell, such as a gel coating, to form the soft gel compositions provided herein. The resulting soft gel compositions provided herein display advantageous properties, such as enhanced bioavailability of the non-polar compounds, for example, after the soft gel has been consumed. Methods for encapsulating non-aqueous pre-gel compositions are well known, and include encapsulation into gelatin shells, or into shells composed of alternative materials or combinations of materials and are known to those of skill in the art. Numerous vendors and companies provide such services and compositions. The soft gels containing the non-aqueous pre-gel concentrates containing the non-polar ingredients provide more bioavailable product compared to tablets or powders. When consumed the soft gel coating dissolves releasing the contents.

Materials for encapsulation are well known. Capsules generally include a high proportion of gelatin (or a gelatin substitute) and additional ingredients, for example a plasticizer, to make the gel soft and pliable. Examples of the capsular materials include, but are not limited to, natural or synthetic gelatin, pectin, casein, collagen, protein, modified starch, polyvinyl pyrrolidone, acrylic, natural or synthetic polymers, cellulose derivatives (such as, but not limited to, hydroxypropyl methylcellulose (HPMC)), and combinations thereof, optionally with one or more plasticizers and/or water. In some examples, soft gel capsules are made to accommodate vegetarian or vegan lifestyles. For such applications, alternative animal-free capsules, such as capsules made from seaweed extract and gluten free starch without modified sugars can be used. Capsular materials also optionally include one or more preservatives, coloring, and opacifying agents, such as titanium dioxide, to decrease the clarity of the shell of the soft gel capsule, flavoring agents and sweeteners, sugars, gastroresistant substances, fillers, binders, lubricants, disintegrants, or combinations thereof. In some embodiments, additional coatings on the capsules, such as immediate release coatings, protective coatings, enteric or delayed release coatings, sustained release coatings, barrier coatings, and combinations thereof can be placed upon the soft gel coating. Flavors and up to 5% sucrose are added in certain examples in order to increase palatability of the soft gel prior to ingestion. In another example, in cases of decreased bioavailability of the non-aqueous pre-gel concentrates containing non-polar ingredients, additional coatings can be added to the soft gel that increase delivery of the non-polar ingredients.

Methods of manufacturing soft capsules are well known to the skilled artisan. Exemplary processes for manufacture of soft capsules include, but are not limited to, the plate process, the rotary die process, the reciprocating die process, the bubble process, and the continuous process (Ebert (1978), “Soft Elastic Gelatin Capsules: A Unique Dosage Form,” Pharm. Tech. 1(5); Reich (2004), “Chapter 11: Formulation and physical properties of soft capsules,” Pharmaceutical Capsules, 2d Ed., Pharmaceutical Press, 201-212; Gullapalli, R. P. (2010) “Soft Gelatin Capsules (Softgels),” J. Pharm. Sci. 99(10):4107-4146). Soft gel capsules can be made using standard rotary die encapsulation technology, detailed in Stanley's Chapter 13 of “The Theory and Practice of Industrial Chemistry” (Lachman, Lieberman, and Kanig, Copyright Lea & Febiger, 1970) and in U.S. Patent Publication No. 2012/0301546 and U.S. Pat. Nos. 6,769,226 and 7,213,511). Capsules can be manufactured and then hermetically sealed to form a one-piece hermetically sealed gelatin shell encasing the concentrate containing the non-polar ingredients. Capsules can also be manufactured according to methods to create seamless capsules (see, e.g., U.S. Pat. Nos. 5,478,508, 5,882,680 and 4,780,316).

The capsules, for example, gelatin capsules, can be manufactured in accord with conventional methods, for example, as a two-piece hard gelatin capsule, sealed or unsealed, typically in standard shape and various standard sizes or as a soft gelatin capsule that is a one-piece hermetically sealed gelatin shell. The shape and size of the capsules can vary in accordance with the method of preparation and the planned use of the capsule. Capsule shapes can be tailored to the requirements of specific products, including, but not limited to, tube-shaped topical disposable capsules, aplicaps for ophthalmic preparations, suppositories, or an oral dosage form. Additional capsule shapes include, but are not limited to, round, oval, tubular, oblong, twist off, or a non-standard shape (e.g., a fish, tree, star, heart, or bear), and are preferably oblong. In some embodiments, non-standard shapes can be used. The size of the capsule used will vary in accordance with the volume of the fill composition, for example the volume of a pre-gel concentrate containing non-polar ingredients, such as the non-aqueous pre-gel concentrates containing non-polar ingredients described herein, that are intended to be contained therein.

The soft gel capsules are filled with non-aqueous pre-gel concentrates provided herein. Compositions for encapsulation by soft gel, such as compositions containing non-polar ingredients, can be formulated in any conventional manner by adding a selected amount of the concentrate, into an acceptable soft gel capsule, such as a soft gelatin capsule.

Soft gel capsule fill volume typically depends upon the density of the encapsulated material, the particular ingredients, and dosages of ingredients thereof desired. A single-body soft gelatin capsule, for example, in oval, oblong or other shapes, typically is provided, for example, in sizes from 3 to 22 US minims (1 US minim being equal to 0.0616 mL or about 1/60 of a fluid dram (about one drop)). A minim is a unit of volume occupied by a fluid or semi-fluid matrix such as, for example, the volume occupied by a concentrate containing non-polar ingredients. For example, an oblong or oval capsule that is 4.0 minims in size can contain 0.246 mL of material, for example, a concentrate containing non-polar ingredients. In another example, an oblong or oval capsule that is 8.0 minims in size can contain 0.493 mL of material, for example, a concentrate containing non-polar ingredients. Hard shell capsule sizes are designated by convention as (000), (00), (0), (1), (2), (3), (4), and (5), with a larger number corresponding to a smaller size. For example, one teaspoon can fill approximately seven size “0” capsules and about five size “00” capsules. Size “00” capsules are generally the largest size utilized for human consumption. Soft gel capsule sizes range from 1 to 120, with the particular size depending on the desired shape and other characteristics of the product. Typically, shapes include, but are not limited to, round, oval, oblong, tube, and special shapes, for example, animals, stars, hearts, or squares. For example, capsules can be oval-shaped and be size 2, 3, 4, 5, 6, 7, 7.5, 8.5, 10, 12, 16, 20, 30, 40, 60, 65, 80, or more. Capsules can be, for example, oblong-shaped and be size 3, 4, 5, 6, 8, 9.5, 11, 12, 14, 16, 20, 22, 24, or more. Other exemplary capsules include round capsules, for example, size 1, 2, 3, 4, 5, 6, 7, 9, 15, 20, 28, 40, 90, or more, and tube capsules, for example, size 5, 6, 8, 17.5, 30, 45, 55, or 120. Capsules can be any color and have any amount of transparency, for example, the capsules can be, but are not limited to, opaque, translucent, or pearlescent capsules.

Soft gel capsules can contain, for example, at least 30, 40, 50, 60, 70, 80, 90. 100, 150, 200, 300, 400, 500, 600, 800 or 1000 mg of the pre-gel composition. Soft gels can contain, for example, at or at about 0.01 mL to at or at about 1 mL, for example, at least 0.01, 0.02, 0.05. 0.1, 0.2, 0.3, 0.4, 0.5, 0.6, 0.7, 0.8, 0.9 and 1 mL of the pre-gel composition. Dosage amount depends upon the active components, a daily dosage thereof and the number of soft gels to be consumed per day. The soft gel composition can provide a single dose or a fractional dose.

The soft capsules can be manufactured, for example, according to the conventional methods listed above with the addition of a printed item on the capsule. Examples of printed items include, but are not limited to, bandings, brandings, designations, identifications, images, and other printed items, and are exerted by methods well known in the art (see, e.g., U.S. Pat. Nos. 2,449,139; 2,623,494; 2,703,047; 2,688,775; 3,124,840; 3,203,347; 3,333,031; and 5,246,635).

A soft gel capsule, for example, a soft gelatin capsule, that has been manufactured in accordance with conventional methods can be characterized by several properties of the capsule, including, but not limited to, Bloom strength and hardness. The Bloom test measures the strength of the gel capsule in which the majority is composed of gelatin, by determining the weight (in grams) of a probe that is needed to deflect the surface of the gel 4 mm without puncturing the surface of the gel. Results of the Bloom test are expressed in Bloom units and generally vary between 30-300 Bloom. The specific procedure of characterization by the Bloom method are well known to the skilled artisan. In other instances, the strength of the capsule wall is measured by an alternative device that assesses the elasticity, integrity and/or strength of the gel capsule, including seam strength. The optimal range for a characteristic soft gel, such as the soft gel capsules described herewith, is 150-250. In addition to Bloom strength, soft gel capsules can also be characterized by their hardness. The hardness of a soft gel capsule is largely determined by the ratio of the plasticizer component to gelatin. Following the drying process during capsule manufacturing, a Bareiss Hardness tester, or an equivalent device, is used to measure the hardness of the soft gel capsule. Measures of soft gel capsule integrity are essential for quality control prior to packaging; moreover, measurements that fall within acceptable ranges, such as values known to the skilled artisan, are used to predict and prevent failure during packaging.

Soft gel capsules, such as the soft gel capsules containing the non-aqueous pre-gel concentrates containing non-polar ingredients provided herein, can be packaged in containers, for example packaging for the soft gels can include, but is not limited to, plastic or glass or metal containers, or blister cards. Soft gel packaging can provide enhanced protection against various insults, including, but not limited to, humidity, oxygen, light, and other toxicities. Plastic containers can be made of any suitable plastic materials including, for example, high density polyethylene, polypropylene, and polyethylene terephthalate, and can also include an integrated or separate desiccant and/or oxygen absorber. Glass bottles can be colored glass, induction sealed, and have a plastic or metal lid. In some embodiments, the packaging of soft gels in bottles can include bottles that are opaque, tamper-resistant, or tamper-evident. In some embodiments, the bottles contain sufficient desiccant material to protect the soft gels from damage from increased water or humidity where the desiccant can be integrated into the container, separate, or as a film.

In some examples, the soft gels composition are packaged in containers. Containers used for packaging soft gels, such as the soft gels containing non-aqueous pre-gel concentrates containing non-polar ingredients provided herein, can be filled with any number of soft gel capsules. For example, a container can contain, for example, 10, 20, 30, 40, 50, 60, 70, 80, 90, 100, 500, 1000, or more than 1000, soft gel capsules, such as the soft gel capsules containing the non-aqueous pre-gel concentrates containing non-polar ingredients provided herein. In other examples, the soft gel compositions are packaged in blister cards. Blister cards used for packaging soft gels, such as the soft gels containing non-aqueous pre-gel concentrates containing non-polar ingredients provided herein, can contain any number of soft gel tablets, e.g., a blister card can hold 3, 5, 10, 20, 30, 40, 50, 60, 70, 80, 90, 100, or more than 100 soft gel capsules, such as the soft gel capsules containing the non-aqueous pre-gel concentrates containing non-polar ingredients provided herein. Blister cards can, for example, contain a foil backing as a barrier. Blister cards or packaging can include, for example, triplex blister film of different types, such as standard and high barrier films, including, for example, triplex Flexafarm Sbc (e.g., PVC 250 my+PE 25 my+PVDC 150 g/mq sbc grade) and Aquaba-PVC (e.g., PVC 250 my+AQUABA 160 g/mq), Aclar, Alu-Alu formats, triple layer blister foil (OPA) with soft tempered aluminium in central position, other layers PVC and polyamide, and new generation multilayer blister combined materials.

b. Formulating the Soft Gel Compositions

The provided non-aqueous pre-gel concentrates contain a high amount of non-aqueous solvent and have displayed advantageous properties after encapsulation, such as encapsulation in a shell or coating, e.g., a gelatin shell or coating, to form a soft gel composition. For example, the soft gel compositions provided herein that contain a high amount of non-aqueous solvent have advantageous properties, such as, for example, the soft gel compositions contain a high amount of non-polar ingredient that does not precipitate out of the composition. Additionally, the soft gel compositions provided herein that contain a high amount of non-polar ingredient display enhanced bioavailability of the non-polar compounds after consumption, for example, human consumption, of the soft gel composition, as compared to tablets or powders.

The non-aqueous pre-gel concentrates provided herein are formulated such that encapsulation of a concentrate, for example, in a coating or shell, e.g., a gelatin coating or shell, yields a soft gel composition that contains a homogenous concentrate, i.e., a concentrate in which the non-polar ingredients are dissolved in the non-aqueous solvent and do not precipitate out. The high solubility of the non-polar ingredients in the non-aqueous solvent leads to advantageous properties, for example, enhanced bioavailability of the non-polar compounds when the soft gel is consumed, for example, by a human.

A number of parameters of the pre-gel concentrates, including ingredients, their relative concentrations, and methods for making the pre-gel concentrates, affect the ability of the non-polar ingredients to remain in solution and not precipitate when a high concentration of non-polar ingredient is present. By extension, these parameters of the pre-gel concentrates also affect the advantageous properties of the soft gel compositions, for example, the bioavailability of the non-polar compounds in the soft gel composition upon consumption, for example, human consumption.

Thus, the pre-gel concentrates are formulated such that after encapsulation, the resulting soft gel composition displays one or more advantageous properties, for example, lack of phase separation and/or precipitation over time, and/or enhanced bioavailability of the non-polar compounds. In one example, the advantageous property is the ability of the provided pre-gel concentrates to yield soft gel compositions that have a lack of precipitation of the non-polar ingredients when encapsulated, for example, in a coating or shell. In another example, the advantageous property relates to the ability of pre-gel concentrates to be encapsulated for human consumption, for example, in a consumable shell or coating. In another example, it can be advantageous that the pre-gel concentrate contains less than or equal to a particular concentration of one or more ingredients. In another example, it can be advantageous that the pre-gel concentrates contains greater than or equal to a particular concentration of one or more ingredients.

c. Ingredients and Concentration Ranges

Each of the provided soft gel compositions contains a pre-gel concentrate encapsulated in a shell or coating, such as a gelatin shell or coating. The pre-gel concentrates, and thus, the encapsulated soft gel compositions provided herein, contain one or more non-polar ingredients, at least one surfactant, and one or more non-aqueous solvents. Typically, encapsulation of a pre-gel concentrate to form a soft gel composition does not change or alter the identity or concentration of the ingredients in the pre-gel concentrate, e.g., the identity and concentration of ingredients in a particular pre-gel concentrate remain the same in the corresponding soft gel composition after encapsulation.

Each of the provided soft gel compositions contains a non-polar ingredient including, but not limited to, the exemplary non-polar ingredients described herein above. Typically, the non-polar ingredient is or has one or more non-polar compounds. The soft gel compositions provided herein can contain one non-polar ingredient or more than one non-polar ingredient, such as two, three, four, five, six, seven, eight, or more non-polar ingredients. The soft gel compositions provided herein can contain high amounts (i.e., concentrations) of non-polar ingredient, such as up to at or about 90 wt % non-polar ingredient.

The soft gel compositions provided herein contain pre-gel concentrates that contain one or more non-polar ingredients, where the total amount of non-polar ingredients is typically present in an amount as a percentage (%) by weight of the soft gel compositions (wt %), e.g., from at or about 40 wt % to at or about 90 wt %, such as between or between about 40% and 45%, 40% and 50%, 40% and 55%, 40% and 60%, 40% and 65%, 40% and 70%, 40% and 75%, 40% and 80%, 40% and 85%, 40% and 90%, 45% and 50%, 45% and 55%, 45% and 60%, 45% and 65%, 45% and 70%, 45% and 75%, 45% and 80%, 45% and 85%, 45% and 90%, 50% and 55%, 50% and 60%, 50% and 65%, 50% and 70%, 50% and 75%, 50% and 80%, 50% and 85%, 50% and 90%, 55% and 60%, 55% and 65%, 55% and 70%, 55% and 75%, 55% and 80%, 55% and 85%, 55% and 90%, 60% and 65%, 60% and 70%, 60% and 75%, 60% and 80%, 60% and 85%, 60% and 90%, 65% and 70%, 65% and 75%, 65% and 80%, 65% and 85%, 65% and 90%, 70% and 75%, 70% and 80%, 70% and 85%, 70% and 90%, 75% and 80%, 75% and 85%, 75% and 90%, 80% and 85%, 80% and 90%, and 85% and 90% non-polar ingredient by weight of the soft gel compositions. Exemplary concentrations of the total amount of non-polar ingredient in the soft gel compositions are at or about 40%, 45%, 50%, 55%, 60%, 65%, 70%, 75%, 80%, 85%, and 90% (wt %) of the soft gel compositions.

Each of the provided soft gel compositions contain pre-gel concentrates that contain at least one surfactant that is a polyethylene glycol derivative of vitamin E, for example, TPGS, TPGS analogs, TPGS homologs and TPGS derivatives described herein. The surfactant typically has an HLB value of between 12 or about 12 and 20 or about 20, for example, 12, 13, 14, 15, 16, 17, 18, 19 or 20, or about 12, about 13, about 14, about 15, about 16, about 17, about 18, about 19 or about 20, typically between at or about 12 and at or about 14. For example, TPGS, such as the TPGS described herein, has an HLB value of about 13.

The polyethylene glycol derivative of vitamin E, e.g., TPGS, is typically present in an amount as a percentage (%) by weight of the soft gel compositions (wt %), e.g., from at or about 5% to at or about 40%, such as 5% to 10%, 5% to 15%, 5% to 20%, 5% to 25%, 5% to 30%, 5% to 35%, 5% to 40%, 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 20% to 40%, 25% to 30%, 25% to 35%, 25% to 40%, 30% to 35%, 30% to 40%, 30% to 30%, 30% to 35%, 30% to 40%, and 35% to 40%, by weight of the soft gel compositions. Exemplary concentrations of the polyethylene glycol derivative of vitamin E, e.g., TPGS, in the soft gel compositions are at or about 5%, 7%, 10%, 12%, 15%, 17%, 20%, 22%, 25%, 27%, 30%, 32%, 35%, 37%, and 40% (wt %) of the soft gel compositions.

The soft gel compositions contain pre-gel concentrates that contain one or more non-aqueous solvents. Typically, the non-aqueous solvents include those that are insoluble or only partially soluble in water. Exemplary non-aqueous solvents include alcohols, such as aromatic alcohols, e.g., benzyl alcohol, alcohol derivatives, such as esters, e.g., benzyl benzoate, and hydrocarbons, such as cyclic hydrocarbons, e.g., d-limonene. In one example, the soft gel composition contains benzyl alcohol as a non-aqueous solvent. In another example, the soft gel composition contains a mixture of benzyl alcohol and d-limonene as the non-aqueous solvents. The soft gel compositions provided herein can contain one non-aqueous solvent or more than one non-aqueous solvent, such as two, three, four, five, or more non-aqueous solvents.

The soft gel compositions provided herein contain pre-gel concentrates that contain one or more non-aqueous solvents, where the total amount of non-aqueous solvents is typically present in an amount as a percentage (%) by weight of the soft gel compositions (wt %), e.g., from at or about 5 wt % to at or about 20 wt %, such as between or between about 5% and 7%, 5% and 10%, 5% and 12%, 5% and 15%, 5% and 17%, 5% and 20%, 7% and 10%, 7% and 12%, 7% and 15%, 7% and 17%, 7% and 20%, 10% and 12%, 10% and 15%, 10% and 17%, 10% and 20%, 12% and 15%, 12% and 17%, 12% and 20%, 15% and 17%, 15% and 20%, and 17% and 20% non-aqueous solvent(s) by weight of the soft gel compositions. Exemplary concentrations of the total amount of non-aqueous solvent(s) in the soft gel compositions are at or about 5%, 7%, 10%, 12%, 15%, 17%, and 20% (wt %) of the soft gel compositions.

d. Exemplary Dosages and Administration of the Soft Gel Compositions

The soft gel compositions provided herein that contain non-aqueous pre-gel concentrates containing non-polar ingredients, can be formulated, for example, for single dosage (direct) administration, multiple dosage administration, or for dilution or other modification. The concentration of the non-polar ingredients in the soft gel compositions is typically effective for delivery of an amount, upon administration, that is effective for the intended treatment or benefit. Those of skill in the art can readily formulate a composition for administration in accord with the methods herein. For example, to formulate a soft gel composition, the weight fraction of a non-polar ingredient is dissolved, suspended, dispersed, or otherwise mixed, for example as detailed in the Examples, below, and then the composition encapsulated by a shell or coating, such as a soft shell or coating, e.g., gelatin, and the resulting soft gel capsule is utilized as a vehicle for delivering an effective concentration of non-polar ingredient. For example, an effective concentration of non-polar ingredient can be delivered via the soft gel to a human, for example, by consumption of the soft gel composition.

The precise amount or dose of the non-polar ingredient administered via the soft gel composition depends upon one or more considerations, including, for example, the condition to be treated, the benefit to be conferred, the weight and general state of the subject, and/or the particular characteristics of the subject. If necessary, a particular dosage and duration and treatment protocol can be empirically determined or extrapolated. For example, exemplary doses of non-polar ingredients, such as the non-polar ingredients provided herein, if necessary, can be used as a starting point to determine appropriate dosages for a particular subject and condition or benefit.

The soft gel compositions containing pre-gel concentrates that contain non-polar ingredients can be formulated so that the dose of non-polar ingredient contained within a single soft gel capsule is from, for example, 100 g or about 100 g to 2000 g or about 2000 g, or 200 g or about 200 g to 2000 g or about 2000 g, such as at or at least about 100 g, 150 g, 200 g, 250 g, 300 g, 350 g, 400 g, 450 g, 500 g, 600 g, 700 g, 800 g, 900 g, 1000 g, 1100 g, 1200 g, 1300 g, 1400 g, 1500 g, 1600 g, 1700 g, 1800 g, 1900 g, or 2000 g, for direct administration.

Typically, the concentration of non-polar ingredient is not more than 90% or about 90% of the weight of the soft gel composition, for example, between 40% or about 40% and 90% or about 90% of the weight of the soft gel composition. For example, when the non-polar ingredient is administered as a component of the pre-gel concentrate encapsulated by a shell or coating, e.g., a gelatin shell or coating, as described herein, the non-polar ingredient is administered at a concentration of between 40% or about 40% and 90% or about 90%, although more dilute or higher concentrations can be used. Generally, the level of non-polar ingredient can be increased or decreased according to the judgment of a person of skill in the art. The amount of the remaining ingredients can be adjusted as needed.

In the methods described herein, soft gel compositions containing non-aqueous pre-gel concentrates containing non-polar ingredients, such as the soft gel compositions described herein, can be administered to a subject for treating a variety of conditions or conferring a particular beneficial effect. In particular, the soft gel compositions provided herein are intended for use in methods in which other pharmaceuticals, nutraceuticals and/or dietary supplements, such as known treatments, can be used to convey a benefit to the subject. The non-polar ingredients contained within the soft gel compositions provided herein are typically included in an amount sufficient to confer a beneficial effect in the absence of undesirable side effects on the subject. The amount of non-polar ingredient to be administered can be determined by one of skill in the art. The precise dosage, which can be determined empirically, can depend on the particular composition, the type of condition to be treated and the seriousness of the condition, the type of benefit to be conferred, in addition to other characteristics known to those of skill in the art.

### C. EXEMPLARY METHODS FOR PREPARING NON-AQUEOUS PRE-GEL CONCENTRATES CONTAINING NON-POLAR INGREDIENTS

Methods for preparing non-aqueous pre-gel concentrates containing a surfactant and high amounts of non-polar ingredients and non-aqueous solvents are provided herein. Equipment for use in the methods and general steps of the methods are described below. The methods include bench-top manufacturing processes, which are used to make small quantities of the concentrates. The methods also include scaled-up manufacturing processes, which are used to make larger batches of the concentrates. Any of the bench-top processes can be scaled up to perform the methods using the scaled-up processes. Any of the provided concentrates can be made using either scaled-up or bench-top processes. The pre-gel concentrates provided herein can be made following the methods provided in U.S. Pat. No. 8,282,977 and U.S. Pub. Nos. 2009-0297491 and 2012-0016026.

1. Equipment Employed in the Methods

Equipment used in various steps of the provided methods for making the pre-gel concentrates can include, for example, vessels, such as tanks, for mixing the ingredients; scales; mixers, for example standard mixers and homogenizers; heating and cooling apparatuses, such as water-jacketed tanks, hot plates, water baths and chillers (coolers), including recirculating coolers; transfer apparatuses, for example, transfer devices, such as, pumps, hoses and sanitary fittings; ball valves; purifiers, for example, filters, such as carbon filters, ion exchange equipment, reverse osmosis equipment, end-point filters and end product filters; evaluation devices, for example, pH and temperature meters; and other equipment. The choice of equipment depends on a plurality of factors, including batch size and the manufacturing process.

a. Scales

One or more scales can be used to measure the amount of the ingredients before adding them to the appropriate vessel. Alternatively, the ingredients can be weighed in the vessel, for example, in a tank on top of a scale.

Any of a plurality of well-known, commercially sold scales can be used to weigh the ingredients. The choice of scale(s) can depend on a number of factors, including the mass of the pre-gel concentrate being made (e.g., the batch size) and the ingredient being weighed. In one example, multiple scales are used to weigh the various ingredients of the pre-gel concentrates. In general, relatively larger capacity (i.e., weight) scale(s) are used in making larger batches of the pre-gel concentrates while relatively smaller capacity scale(s) are used in making smaller batches.

Exemplary of the scales used to weigh the ingredients using the provided methods are a Toledo Scale (Model GD13x/USA); a Sartorius Basic Analytical Scale (Model BA110S), which is a basic series analytical scale with a 110 g capacity and a resolution of 0.1 mg; and an OHAUS Scale (Model CS2000), which is a compact portable digital scale having a 2000 g capacity and a resolution of 1 g.

b. Purifiers

Purifiers, such as filters, are used in the provided methods to remove impurities from the ingredients prior to their addition to and/or from the concentrates. Purifiers that can be used with the provided methods include filters, for example, 100 micron filters and carbon filters, which are filters that use activated carbon to remove impurities by chemical adsorption. Other filters are well known and can be used with the provided methods. The purifiers also include reverse osmosis purifiers, which use mechanical pressure to purify liquids. The purifiers also include exchange purifiers, for example, an ion exchange purifier. The ion exchange purifier can use a resin bed, such as a zeolite resin bed, to replace salts, such as cations, e.g., magnesium and calcium, with other cations, such as sodium and potassium cations. Such purifiers can be purchased, for example, from Aqua-Pure Filters (Clarkston, Mich.). In one example, the purifier is an end product filter (e.g., a 100 micron filter; Product No. BPEM 100-5GP; FSI, Michigan City, Ind.). This filter can be used to filter any impurities out of the final product (e.g., the final pre-gel concentrate). Other filters also are known and can be used with the provided methods.

c. Vessels

One or more vessels can be used in the methods to contain the ingredients of the provided pre-gel concentrates, for example, during mixing and/or heating or cooling. The vessels can be tanks, for example, water-jacketed tanks; pots; and/or beakers, for example, Pyrex® beakers. Additional vessels, for example, holding and/or packaging tanks, can be used for holding and/or packaging the concentrates and/or for addition/mixing of additional ingredients to the concentrates.

A number of vessels are available for mixing ingredients. Typically, the vessels are cleaned, for example, rinsed, soaped and/or sanitized, according to known procedures prior to use and between uses, such as with the cleaning procedures described below.

In the bench-top process, the vessel can be a container, for example, a bench-top container, such as a flask, beaker (e.g., a Pyrex® beaker), vial, measuring container, bottle and/or other bench-top container.

In the scaled-up manufacturing process, the vessels can be tanks Typically, the tanks are equipped with one or more mixers, for example, a standard mixer and/or homogenizer, which are used to mix the ingredients that are added to the tank. In one example, the tank is further equipped with a heating and/or cooling device. For example, the tank can be a water-jacketed tank. The temperature of the water-jacketed tank is controlled through the water jacket, for example, to heat the contents, such as during mixing.

Exemplary of the tanks that can be used with the provided methods are water-jacketed tanks, for example, the Overly 550 gallon water-jacketed tank (Model 10576501G), which has a 550 gallon capacity, the Schweitzer's 450 gallon tank (Model #5214-C), which has a 450 gallon capacity, and the Royal 190 gallon water-jacketed tank (Model 9977-5), which has a 190 gallon capacity. Other tanks are well known and can be used with the provided methods for mixing the pre-gel concentrates.

d. Mixers

Mixers are used in the methods to blend or mix the ingredients and pre-gel concentrates. In some examples, the mixers can be used to keep the ingredients and/or concentrates circulating to maintain temperature, viscosity and/or other parameters of the mixture. Suitable mixers include, but are not limited to, standard mixers, for example, those that can be used to mix ingredients and maintain a homogeneous mixture, such as while heating a mixture of ingredients. Exemplary of the standard mixers are LIGHTNIN® mixers (LIGHTNIN, Rochester, N.Y.), for example, Model Numbers XJC117 and ND-2. In one example, the LIGHTNIN® mixers are fixed-mount, gear drive high-flow mixers, for use with closed tanks Another example of a standard mixer is a mixer sold by IKA®, for example, overhead IKA® mixers. Exemplary IKA® mixers include Model Nos. RW-14 Basic and RE-16S, which are laboratory stirrers that can be used to mix ingredients. In some examples, the mixer can be attached to the vessel, e.g., the tank, such as by mounting or clamping onto the tank, such as at the top of the tank. In other examples, the mixer can be placed in the vessel for mixing.

The mixer can be a homogenizer which can be used, for example, to form homogenous mixtures. The homogenizer provides high-shear dispersion of solids and emulsification of immiscible liquids at high shear rates. Suitable homogenizers include, but are not limited to, high-shear homogenizers, for example, reversible homogenizers sold by Arde Barinco, Inc. (Norwood, N.J.). Exemplary Arde Barinco, Inc. reversible homogenizers are Model CJ-50 (a 3600 rpm mixer having a 6-inch rotor diameter, tip speed of 5575 ft/minute, emersion depth of 33 inches, and six separate openings at the bottom and top, which concentrate the liquid into six chambers, reducing the surface volume and creating a shear effect); and Model CJ-4E (a 10,000 rpm mixer with fan-cooled motor, optimized for 1 to 5 gallon batch sizes, having a 1.875 inch rotor diameter, tip speed of 4920 rpm, and immersion depth of 16 inches). The homogenizers further include other homogenizers, for example, other reversible homogenizers sold by Arde Barinco, Inc.

In one example, the homogenizer is attached to the top of the vessel, for example, the tank, for example, by clamps or by channel locks and an electrical hoist. In another example, the homogenizer is placed in the vessel. The Arde Barinco reversible homogenizers contain axial flow impellers, which create two distinct mixing actions, depending on direction. Downward “vortex flow” pulls solids from the top and bottom of the mixture, while upward “umbrella flow” controls mixing at the highest shear and recirculation rates without splashing or incorporating air. The reversible homogenizers typically are equipped with an adjustable baffle plate, which can be adjusted to control the type of mixing, for example at different times during mixing.

A number of other mixers are well known and can be used with the provided methods. Exemplary of suitable mixers that can be used with the provided methods are homogenizers, inline mixers, ribbon mixers, plow mixers, paddle mixers, Forberg® mixers, conveyors, bag dumps and compactors, V-blenders, blade mixers, double cone mixers, continuous mixers, speedflow mixers, batch mixers, double ribbon blenders, paddle and ribbon mixers with choppers, plow blenders, turbulent mixers, fluidizing Forberg-type mixers, air mixers, active mixers, passive mixers, top-entry mixers, side-entry mixers, static mixers, fixed-entry mixers, portable mixers (e.g., direct and gear drive), sanitary mixers, drum mixers, bulk container (IBC) mixers, lab stirrers, variable speed mixers, dough mixer, vertical mixer, spiral mixer, twin arm mixer, fork mixer, double spiral mixer, all agitators, agitator mixers, Banbury® mixers, rubber mixers, Blondheim mixers, churn mixers, conical mixers, continuous mixers, disperser mixers, pan mixers, emulsifier mixers, Hobart® mixers, liquefier mixers, Littleford mixers, meat mixers, plow mixers, Mix-Muller® Mixers, vertical screw mixers (e.g., Nauta mixers), Oakes mixers, planetary mixers, pony mixers, pug mixers, Ross mixers, rotary mixers, Sigma mixers, single arm mixers, tote bin mixers, tumble mixers, vacuum mixers, Turbolizer mixers, twin shell mixers, V-type mixers, zigzag mixers, side-arm mixers, hand-held mixers, stir rods, stir bars, magnetic mixers, overhead mixers (e.g., mechanical and/or electric overhead mixers), and any mixer known to those of skill in the art.

e. Heating/Cooling Apparatuses

Equipment that can be used in the methods includes heating and cooling apparatuses. The heating and cooling apparatuses can be used to control the temperature of the ingredients and combinations thereof, such as while generating the pre-gel concentrates.

Heating apparatuses that can be used in the provided methods are those that are capable of heating the mixture to between at or about 45° C. and at or about 85° C., for example, to at or about 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C. or 85° C. Typically, the heating apparatus is used to heat the mixtures to a temperature of at or about 60° C.

The heating apparatus can be a water jacket, for example, a water jacket on a water-jacketed tank, which can be controlled, for example, by a control panel, such as to adjust the temperature of the contents of the tank. Other suitable heating apparatuses are immersible and/or submersible heaters, for example, 12 KW or 13 KW sanitary heaters, including food-grade heaters, that can be immersed into the tanks, typically while mixing and typically when higher temperatures are required, such as when temperatures greater than 60° C. or about 60° C., or greater than 80° C. or about 80° C. are required. The heating apparatuses also include stoves, for example, propane stoves, and hot plates, for example, Thermolyne® hot plates (e.g., Model Nos. 846925 and SP46615).

The cooling apparatus can be any apparatus that can cool the ingredients and combinations thereof, such as rapidly cooling and/or cooling while mixing the ingredients. Typically, the cooling apparatus is capable of cooling the mixtures to a temperature between at or about 25° C. and at or about 45° C., for example, to at or about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C. or 45° C. In some examples, the cooling apparatus can cool the mixture to a temperature between at or about 30° C. and at or about 35° C. Typically, the cooling is rapid cooling. For example, the concentrates can be cooled to a temperature between at or about 30° C. and at or about 35° C. in at or about 15 minutes to at or about 2 hours, for example, in at or about 30 minutes to at or about 60 minutes, such as in at or about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes. In an exemplary method, the concentrates can be cooled to a temperature between at or about 30° C. to at or about 35° C. in at or about 30 minutes to at or about 60 minutes.

Suitable cooling apparatuses for used in the methods include chillers, for example, recirculating coolers. The cooling apparatuses can be attached to the vessel, such as remotely or by a tank mounted in the cooler, to repeatedly circulate fluid from the tank, through the chiller and back to the vessel, to rapidly cool and maintain the temperature of the mixture during mixing. Exemplary of cooling apparatuses that can be attached to the tank and used with the provided methods are open-loop chillers and closed-loop chillers, for example, those sold by Turmoil (West Swanzey, N.H.), such as Model No. OC-1000 RO. Suitable cooling apparatuses also include water baths and ice baths, for example, water baths and/or ice baths in which the vessel is placed, for example, during homogenizing. Other cooling apparatuses are well known by those of skill in the art and can be used with the provided methods.

f. Transfer Devices

Transfer devices can be used with the provided methods to transfer liquid from one vessel to another vessel. Transfer devices include, for example, transfer pumps and associated accessories (e.g., fittings), including ball valves, sanitary fittings (for example, sanitary fittings sold by Granger, Inc. (Lake Forrest, Ill.)) and transfer hoses (for example, hoses sold by Sani-Tech West (Oxnard, Calif.)), such as food grade hoses attached to a transfer pump, for example, the food grade Sani-Tech® STHT-R-HD Braid-Reinforced Heavy Duty Silicone Hose. Suitable transfer pumps include the Teel Pump (Model 2P377B; Granger, Inc., Lake Forrest Ill.), a self-priming pump having a power rating of 2 HP, 60 Hz voltage, 208-230/460 AC, speed of 3450 rpm; and other pumps, such as self-priming pumps from Granger, Inc. The transfer device can also include equipment for manually transferring the liquid to another vessel, for example, by pouring, pipetting and/or other well-known methods of manually transferring liquids.

g. Evaluation Equipment

Evaluation equipment includes equipment that can be used to evaluate properties of the pre-gel concentrates, such as the temperature, pH, clarity, color, activity, smell and/or taste of the products. Suitable evaluation equipment includes pH and temperature meters, such as the pH and temperature meter sold by Hanna Instruments (Model No. HI 8314; Ann Arbor, Mich.), which can be used to measure the temperature and the pH of the concentrate. Temperature meters can also include temperature probes, for example, digital and/or water-proof temperature probes, such as temperature probes sold by Cooper-Atkins (Middlefield, Conn.), for example, the Cooper-Atkins digital waterproof temperature probe (Model # DPP400W). The products can be evaluated and analyzed to verify the amounts of the non-polar ingredients and to verify that the concentrates meet industry standards, such as to verify that the products do not contain levels of microbials and heavy metals that are above acceptable levels. Typically, these tests are performed by sending a sample of the product to a commercial testing facility, such as Eurofins U.S. (Des Moines, Iowa) or Advanced Botanical Consulting & Testing, Inc. (Tustin, Calif.), or any other facility capable of performing such tests.

2. General Methods for Preparing Non-Aqueous Pre-Gel Concentrates

In general, the methods useful for making the pre-gel concentrates provided herein are performed by combining the ingredients, i.e., non-polar ingredients, non-aqueous solvents, and a surfactant, and mixing to form a homogenous pre-gel concentrate. The pre-gel concentrate typically is generated in a vessel. The vessel can be, for example, a tank. Typically, the mixture is heated to a desired temperature to form a homogenous mixture. For example, the mixture can be heated to 60° C. The provided methods can include additional steps. In some examples, the additional steps include evaluating properties of the pre-gel concentrates, adding additional ingredients, packaging and/or filtering.

The provided methods can be performed using a bench-top manufacturing process (for small batch sizes) or performed using a scaled-up manufacturing process (for larger batch sizes). Each of the provided pre-gel concentrates can be made with either the bench-top or scaled up process. In one example, the pre-gel concentrate is first made with the bench-top process and then the method is scaled up to make larger quantities of the product.

The bench-top process can be performed on a bench, counter, table or any other suitable surface. Typically, the bench-top process is used to make pre-gel concentrates having relatively smaller volumes than those made with the scaled-up process. For example, volumes less than 1 L or about 1 L, or less than 1 gallon or about 1 gallon, for example, less than or about 500 mL, for example, less than or about 1000 mL, 900 mL, 800 mL, 700 mL, 600 mL, 500 mL, 450 mL, 400 mL, 350 mL, 300 mL, 250 mL, 200 mL, 150 mL, 100 mL, or 50 mL or less, can be made using the bench-top process.

For the bench-top process, the equipment can be sufficiently compact to be used on a bench-top or other similar surface, and can be sufficiently compact to be moved, for example, lifted, by the artisan using the methods. For example, the vessels can be bench-top vessels. Exemplary bench-top vessels include, for example, flasks, beakers, vials, measuring containers, bottles and/or other bench-top containers. In some examples, the vessel in the bench-top process is a Pyrex® beaker.

Typically, the mixers for use in the bench-top processes of the provided methods are mixers that can be used in the bench-top vessels. Mixers that can be used in the bench-top vessels include, for example, standard mixers, such as hand-held mixers, stir rods, stir bars, magnetic mixers and overhead mixers, including, for example, mechanical and/or electric overhead mixers, and any other mixer that is suitable for use in the bench-top vessel. Exemplary standard mixers include those sold by IKA®, for example, overhead IKA® mixers, such as Model Nos. RW-14 Basic and RE-16S, which are laboratory stirrers and can be used to mix ingredients, such as to for a homogenous concentrate. Suitable bench-top mixers also include homogenizers, for example, reversible homogenizers. An exemplary reversible homogenizer is the Arde Barinco reversible homogenizer, Model no. CJ-4E.

Typically, the heating and cooling apparatuses are those that can be used with the bench-top vessels, such as hot plates, ice baths and/or water baths, into (or onto) which the vessels can be placed, for example, for rapid cooling. The evaluation device used in the bench-top process, for example, the temperature and/or pH meters, typically are capable of being placed in the bench-top vessels.

The scaled-up manufacturing process of the methods typically is used to make pre-gel concentrates of relatively larger volumes, such as volumes greater than 1 L or about 1 L, or greater than 1 gallon (gal) or about 1 gallon. For example, volumes greater than or about 0.5 L, for example, greater than or about 0.5 L, 1 L, or 2 L, or greater than or about 1 gal, 2 gal, 3 gal, 4 gal, 5 gal, 6 gal, 7 gal, 8 gal, 9 gal, 10 gal, 11 gal, 12 gal, 13 gal, 14 gal, 15 gal, 16 gal, 17 gal, 18 gal, 19 gal, 20 gal, 21 gal, 22 gal, 23 gal, 24 gal, 25 gal, 26 gal, 27 gal, 28 gal, 29 gal, 30 gal, 40 gal, 50 gal, 60 gal, 70 gal, 80 gal, 90 gal, 100 gal, 150 gal, 200 gal, 250 gal, 300 gal, 350 gal, 400 gal, 450 gal, 500 gal, 550 gal, 600 gal, 650 gal, 700 gal, 800 gal, 900 gal, or 1000 gal or more, can be made using the scaled-up manufacturing process.

In general, equipment used for the scaled-up process is compatible with larger volume batches (batch sizes). For example, the vessels for use in the scaled-up processes can be tanks, for example, water-jacketed tanks, which are equipped with water jackets that can be used as heating apparatuses to ingredients during generation of the pre-gel concentrate. The water jackets typically are controlled via control panels. The transfer device can include devices attached to and connecting the tanks, such as transfer pumps and associated fittings, for example, ball valves and hoses that are attached to the tanks Mixers for use in the scaled-up process can be standard mixers, for example, mounted mixers, such as LIGHTNIN® mixers, e.g., Model Nos. XJC117 (a fixed-mount, gear drive high-flow mixer) and ND2.

Prior to beginning the methods, the water jacket lines on any water-jacketed oil phase and water phase tank can be bled. The water jacket switches can then be turned on to maintain a pressure in the water jackets of between at or about 20 psi and at or about 40 psi (pounds per square inch). If the pressure in the water jacket falls below 20 psi during the method, the line can be bled and checked for bubbles while purging the line.

a. Ingredients

The pre-gel concentrates include a surfactant, for example, a polyalkylene glycol derivative of vitamin E, e.g., TPGS, the non-polar ingredients, for example, non-polar ingredients that are or contain non-polar compounds, and non-aqueous solvents. Typically, the ingredients do not include aqueous ingredients, e.g., water. Exemplary of ingredients used in the provided pre-gel concentrates are non-polar ingredients, for example, non-polar ingredients that are or contain non-polar compounds, including any of the non-polar ingredients or compounds provided herein; surfactants, including any of the polyalkylene glycol derivatives of vitamin E provided herein; and non-aqueous solvents, for example, solvents that have no or only partial solubility in water.

b. Production of the Non-Aqueous Pre-Gel Concentrates

Typically, the ingredients are weighed and/or measured, for example, using one or more scales (e.g., one or more of the scales described herein), before they are added to the mixing vessel (e.g., any vessel described herein). In one example, the amount of each ingredient to be added is determined according to the provided methods for formulating the pre-gel concentrates. Typically, the desired concentration, by weight (w/w), of the final pre-gel concentrate is used to calculate the amount of each ingredient that is added to the vessel. Alternatively, the desired volume per weight, volume per volume or weight per volume can be used to calculate the correct amount of an ingredient to be measured and added to the vessel.

The ingredients can be added simultaneously and/or sequentially, for example, in any order or in a specific order. In one example, one or more ingredients are added first and heated, prior to addition of further ingredient(s). Typically, when the ingredients include a surfactant, a non-aqueous solvent and a non-polar ingredient, the ingredients are added sequentially, in the following order: 1) surfactant; 2) non-aqueous solvent; 3) non-polar ingredient. Alternatively, the ingredients can be added in a different order, for example, any order. Two or more ingredients can be added simultaneously.

Typically, when the oil phase includes a surfactant, particularly when the surfactant is a surfactant that is solid at room temperature, for example, a tocopherol polyethylene glycol succinate surfactant, the surfactant is the first ingredient added to the vessel. Typically, the non-polar ingredients are the last ingredients added to the vessel.

Typically, in order to dissolve the ingredients, the ingredients are mixed in the mixing vessel using a standard mixer (e.g., any of the standard mixers described herein) and heated using a heating apparatus (e.g., any of the heating apparatuses described herein). Typically, the ingredients are heated such that the ingredients reach an elevated temperature, for example, between about 45° C. or about 45° C. and 85° C. or about 85° C., for example, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85° C., typically, 60° C. or about 60° C. Typically, the ingredients are heated at an elevated temperature, for example, at 60° C. or about 60° C., until the ingredients dissolve, e.g., until a homogenous mixture is formed.

The ingredients typically are homogenized, continuously or intermittently, until the ingredients become homogeneous at the elevated temperature, for example, at between about 45° C. or about 45° C. and 85° C. or about 85° C., for example, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85° C., typically, 60° C. or about 60° C. In one example, the baffle plate of the mixer is adjusted, for example, by moving the baffle plate further down into the mixture or further up out of the mixture, to control the type of mixing, for example, to switch from downward flow to upward flow and vice versa, during mixing of the ingredients. In another example, the homogenizer can be adjusted to increase or decrease shear or to maintain the shear at a particular speed. Methods for homogenizing ingredients are well known and other methods can be used to homogenize in the provided methods.

Typically, after all the ingredients have been added and made homogeneous, the pre-gel concentrate is filtered using an end-product filter (e.g., a 100 micron end-product filter), to remove any impurities.

c. Transfer and/or Packaging

After mixing, the pre-gel concentrate is transferred, using one or more transfer means, to another vessel, for example, a holding or packaging vessel and/or a storage container. Any transfer means can be used. For example, any means for transferring the contents of one vessel to another vessel as described above, for example, transfer pumps and associated equipment, for example, sanitary fittings, hoses and/or ball valves; and manual transfer means, for example, pouring and/or pipetting means or other known transfer means. In some examples, the mixture is kept clean, for example, sterile during transfer, for example, by using transfer means with sanitary fittings and/or combining the phases in a sterile environment.

In one example, the mixture is transferred to a holding tank. In another example, the pre-gel concentrate, after being made and filtered, is transferred, e.g., by hot filling while the concentrate is a liquid, to a storage container, e.g., a vial, plastic bottle, or bag-in-α-box type packaging. Typically, the concentrate is allowed to cool naturally in the storage container. Alternatively, a cooling apparatus, e.g., a refrigerator, freezer or water bath, can be used to cool the concentrate in the storage container. The pre-gel concentrate can remain a liquid as it cools or, alternatively, can solidify or partially solidify as it cools in the storage container, e.g., becomes a waxy solid.

### D. EXAMPLES

### Example 1

### A. Method of Producing TPGS Compositions

d-α-Tocopheryl polyethylene glycol 1000 succinate (TPGS 1000) was synthesized from vitamin E succinate according to the following general procedure. See also, U.S. patent application Ser. No. 14/207,310 and International Patent Application No. PCT/US14/25006, now published as US-2014-0271593-A1 and WO 2014/151109, respectively.

Polyethylene glycol (PEG) 1000 (168.7 kg) was added to a reaction flask containing 1430 L of toluene, followed by the addition of 71.5 kg of vitamin E (α-tocopheryl acid) succinate and 2.86 kg of p-toluene sulfonic acid. The reaction mixture was heated to 110-112° C. and refluxed for up to 6.5 hours, removing the water formed during the esterification reaction by azeotropic distillation. The reaction was terminated when the desired amounts of TPGS monomer and TPGS dimer were formed, as indicated by high performance liquid chromatography (HPLC) and thin layer chromatography (TLC), resulting in the TPGS compositions set forth in Table 1a below. Each TPGS composition in Table 1a was formed by terminating the reaction at a different time point, up to 6.5 hours, and contained various amounts of TPGS monomer and TPGS dimer. The remainder of the TPGS composition was made up of unreacted starting materials, such as vitamin E and PEG The reaction was terminated by cooling the reaction mixture to room temperature, followed by washing with 25 L of a 10% solution of NaHCO₃. The solution stirred for 10 minutes, and after stirring was allowed to separate into layers. The organic (toluene) layer was removed, 6 kg of activated carbon (charcoal) was added, and the solution was heated to 55-60° C. and maintained at this temperature for 1 hour. The solution was then cooled to room temperature, filtered through 10 kg of Celite® Hyflo® filter aid (Sigma Aldrich, St. Louis, Mo.) and then washed with 100 L of toluene. The filtered toluene solution was concentrated by vacuum distillation below 60° C. to remove the toluene. Water (140 L) was added to remove traces of toluene and was then removed via vacuum distillation below 60° C. to obtain ˜180 kg of a crude α-tocopheryl polyethylene glycol 1000 succinate composition that contained a mixture of TPGS monomer and TPGS dimer, along with unreacted PEG 1000 and α-tocopherol.

TABLE 1a
Amounts of TPGS monomer and TPGS
dimer formed during reaction

| | | | Total |
|---|---|---|---|
| TPGS | Monomer | Dimer | (% monomer + |
| composition | (%) | (%) | % dimer) |
| 1 | 43.90 | 53.90 | 97.80 |
| 2 | 42.80 | 48.80 | 91.60 |
| 3 | 40.95 | 53.15 | 94.10 |
| 4 | 43.52 | 49.80 | 93.32 |
| 5 | 55.88 | 29.27 | 85.15 |
| 6 | 52.92 | 33.70 | 86.62 |
| 7 | 42.76 | 51.10 | 93.86 |
| 8 | 40.39 | 54.90 | 95.29 |
| 9 | 57.70 | 40.40 | 98.10 |
| 10 | 39.35 | 35.56 | 74.91 |
| 11 | 60.00 | 38.10 | 98.10 |

A series of extractions were performed on the crude TPGS composition. The crude TPGS composition (˜180 kg) was dissolved in 360 L of methanol and then 540 L of cyclohexane was added. The solution was stirred and then allowed to separate into layers. The cyclohexane layer was removed and an additional 540 L of cyclohexane was added to the remaining methanol layer. The solution was stirred and then allowed to separate into layers. The cyclohexane layer was again removed and an additional 540 L of cyclohexane was added to the remaining methanol layer. The solution was again stirred and allowed to separate into layers. The cyclohexane layer was removed, and the remaining methanol layer was further diluted with an additional 270 L of methanol. Activated carbon (18 kg) was added and the solution was heated to 55-60° C. and maintained at this temperature for 1 hour. The solution was then cooled to room temperature, filtered through 30 kg of Celite® Hyflo® filter aid, and washed with 100 L of methanol. The methanol solution was passed through a micron filter, then concentrated via vacuum distillation below 60° C. to obtain ˜98-102 kg of a TPGS composition. All traces of solvent were then removed by purging with nitrogen at 55° C. for two hours to obtain ˜98-102 kg of a purified TPGS composition that contained TPGS monomer and TPGS dimer.

One typical batch of TPGS prepared to contain a high dimer concentration, and used in the Examples below, had the following components:

TPGS monomer: 48%
TPGS dimer: 51%

### Vitamin E: 0.42%

Vitamin E succinate: 0.46%.

Other typical batches contained:

TPGS monomer: 46.09%-43.15% w/w
TPGS dimer: 39.07%-50.28% w/w
Other: up to about 3%-3.2% w/w

For example, the batches used in Example 11, below, contained:

TPGS monomer: 46.55%-48.72% w/w
TPGS dimer: 46.88%-47.33% w/w
Other: up to about 3.95%-6.55% w/w

### B. Evaluation of the Clarity of the TPGS-Containing Compositions by a Turbidity Analysis

The clarity of the TPGS compositions prepared above was evaluated by a turbidity analysis. TPGS compositions 1-11 were formulated as 1 g concentrates and each were dissolved in 8 oz. of water. The resulting aqueous liquid dilution compositions then were evaluated for clarity by measuring turbidity using a nephelometer. Results of the evaluation are set forth in Table 2 below.

Each of the eleven TPGS compositions listed in Table 1a above was diluted in water (purified according to the provided methods) using the following steps.

Eight ounces of water was heated in a Pyrex® beaker by placing the beaker on a Thermolyne hot plate (Model #846925) until the water reached 49.8° C. The TPGS composition concentrate was then added to the heated water and stirred with a stir rod until dispersed. The resulting aqueous TPGS composition was cooled to room temperature (about 25° C.). The cooled aqueous TPGS composition was added to an amber-glass screw-top vial (Alcon) for evaluation.

The vials containing the aqueous TPGS compositions were sent to ACZ Laboratories, Inc. (Steamboat Springs, Colo.) for turbidity analysis using a nephelometer. Results are listed in the form of Nephelometric Turbidity Units (NTU) and are indicated in Table 1b below.

TABLE 1b
Turbidity (NTU) of aqueous TPGS compositions

| | | | Total | |
|---|---|---|---|---|
| TPGS | Monomer | Dimer | (% monomer + | Turbidity |
| composition | (%) | (%) | % dimer) | (NTU) |
| 1 | 43.90 | 53.90 | 97.80 | 8 |
| 2 | 42.80 | 48.80 | 91.60 | 8.2 |
| 3 | 40.95 | 53.15 | 94.10 | 10 |
| 4 | 43.52 | 49.80 | 93.32 | 10 |
| 5 | 55.88 | 29.27 | 85.15 | 14 |
| 6 | 52.92 | 33.70 | 86.62 | 14 |
| 7 | 42.76 | 51.10 | 93.86 | 18.5 |
| 8 | 40.39 | 54.90 | 95.29 | 39.4 |
| 9 | 57.70 | 40.40 | 98.10 | 71 |
| 10 | 39.35 | 35.56 | 74.91 | 80 |
| 11 | 60.00 | 38.10 | 98.10 | 80 |

### Example 2

### Preparation of Non-Aqueous Pre-Gel Concentrates Containing TPGS, Benzyl Alcohol or Benzyl Alcohol and d-Limonene and Non-Polar Ingredients

Non-aqueous pre-gel concentrates were prepared according to the procedure described below and containing the ingredients listed below. The non-aqueous pre-gel concentrates contained α-tocopheryl polyethylene glycol succinate (TPGS), benzyl alcohol or benzyl alcohol and d-limonene, and one or more non-polar ingredients. After formation of the non-aqueous pre-gel concentrates, the compositions were encapsulated in a collagen-containing shell to form a soft gel.

A. Ingredients

The non-aqueous pre-gel concentrates contained between 26.96% and 34.6% TPGS and between 7.49% and 15.42% benzyl alcohol or benzyl alcohol and d-limonene. The remainder of the concentrates, between 54.8% and 65.55%, contained one or more non-polar ingredients.

Ingredients in the non-aqueous pre-gel concentrates included:

TPGS, a surfactant, that contained a high amount of the dimer form (e.g., 85%-90%) and a low amount of the monomer form (e.g., 10%-15%), such as the TPGS described in Example 1, above. See also, U.S. patent application Ser. No. 14/207,310 and International PCT Application No. PCT/US14/25006, now published as US-2014-0271593-A1 and WO 2014/151109, respectively;

one or more non-aqueous solvents, including benzyl alcohol (Sigma Aldrich, St. Louis, Mo.) and d-limonene (99% GRAS-certified; Florida Chemical, Winter Haven, Fla.); and

one or more non-polar ingredients, including: vitamins, including vitamin E oil (such as the vitamin E oil sold under the name Novatol™ 5-67 (D-alpha-tocopherol) by ADM Natural Health and Nutrition, Decatur, Ill.), vitamin D3 (1.0 million IU/gram; DSM, Parsippany, N.J.) and vitamin K2 (as MK-7; NattoPharma®, Metuchen, N.J.); a borage oil compound that contains not less than (NLT) 22% of the non-polar ingredient C18:3 gamma-linolenic acid (GLA) (such as the borage oil sold by Sanmark Limited, Dalian, Liaoning Province, China); alpha-lipoic acid (such as sold by NutriChem Resources Company, Walnut, Calif., and Zhejiang Medicines & Health Products Import & Export Co., Ltd., Hangzhou, China); ubiquinol (Kaneka Ubiquinol®, sold by Kaneka Nutrients, Pasadena, Tex.); pyrroloquinoline quinone (PQQ; such as PureQQ, sold by Nascent Health Science, Allentown, N.J.); carotenoid-containing compounds, including astaxanthin (such as AstraREAL®, sold by Fuji Health Science, Burlington, N.J.; AstaPure®, sold by Alga Technologies, Hevel Eilot, Israel; BioAstin®, sold by Cyanotech, Kailua-Kona, Hi.); fatty acids and fatty acid derivatives, including an oleic acid compound that contains 70% oleic acid (KIC Chemicals, Inc., New Paltz, N.Y.) and cetyl myristoleate, an esterified derivative of myristoleic acid (sold as Myristin®, EHP Products, Inc., Mt. Pleasant, S.C.); a phospholipid, lecithin (phosphatidylcholine; such as Lecithin Ultralec® P, ADM Natural Health and Nutrition, Decatur, Ill.; and Phospholipon®, a lecithin fraction with approximately 95% phosphatidylcholine, sold by Lipoid, Steinhausen, Switzerland, and American Lecithin Company, Oxford, Conn.); a turmeric/curcumin composition that contains 95% curcumin (sold by Siddharth International, Mumbai, India); Perluxan™, a hops (Humulus lupulus L.) extract that contains a minimum of 30% alpha acids (including humulone, co-humulone, adhumulone, iso-cohumulone and iso-adhumulone) and 10% beta acids (lupulone and colupulone) (sold by Pharmachem Laboratories, Kearny, N.J.); and ApresFLEX®, a boswellia extract that contains acetyl-11-keto-β-boswellic acid (AKBA; sold by PLT Health Solutions, Morristown, N.J.).

B. Preparation of the Non-Aqueous Pre-Gel Concentrates

The non-aqueous pre-gel concentrates were made by a bench-top process according to the methods described below. Alternatively, the concentrates can be prepared by scaling up the bench-top process to make larger batch sizes of the non-aqueous pre-gel concentrates.

For each non-aqueous pre-gel concentrate, the appropriate amount of each ingredient was weighed using a Toledo Scale (Model GD13x/USA), Sartorius Basic Analytical Scale (Model BA110S), an OHAUS Scale (Model CS2000) or a CTS 6000 Scale (Model CTS-6000). Selection of the scale was dependent on the weight of each ingredient being weighed.

The appropriate amounts of TPGS, benzyl alcohol, and d-limonene (when indicated) were added to a vessel (e.g., a Pyrex® beaker) and heated to 60° C. using a Thermolyne hot plate while mixing with a standard mixer (IKA® Model No. RE-16 S1, an overhead mixer (laboratory stirrer) compatible with the bench-top process). Once the initial ingredients dissolved and formed a homogeneous mixture, the non-polar ingredients were added. The ingredients then were homogenized by placing a reversible homogenizer (Arde Barinco, Inc., Model No. CJ-4E) in the vessel (beaker) and turning it on at 850-1200 rpm. Mixing with the homogenizer continued while maintaining the temperature at 60° C. using the hot plate until the mixture became homogenous. During mixing, the baffle plate on the homogenizer was adjusted to achieve and maintain an emulsion, for example, by moving the baffle plate further into and/or out of the ingredient mixture.

The composition then was filtered, using a 100-400 micron filter and then packaged (transferred) by filling into one or more storage containers, such as plastic bottles or 5 gallon pails, where it was cooled to room temperature (about 25° C.). Alternatively, the mixture can be packaged into a bag-in-α-box type storage container. Each of the non-aqueous compositions was a liquid at room temperature.

### C. Preparation of Soft-Gel Capsules

Soft gel capsules were prepared by a service provider (Captek Softgel International, Cerritos, Calif.) from a gelatin or other suitable material using standard methods. The outer capsule is formed, for example, using a polar emulsion and gelatin or other known gel replacement, at an appropriate ratio to form the gel capsule. The capsule is of a size to encompass a dosage amount for direct oral consumption and is filled with an appropriate selected dosage of a composition as described above.

### Example 3

### Exemplary Pre-Gel Concentrates

Exemplary pre-gel concentrates are provided below. These were introduced into soft gel capsules to form soft gel compositions. Tables 2-5, below, set forth the ingredients in the non-aqueous pre-gel concentrates, the grams of each ingredient per batch, the amount (mg) of each ingredient per 0.5 mL serving, and the percentage by weight (wt %) of each ingredient by weight of the composition. The non-aqueous compositions were prepared according to the provided methods using a bench-top process, described above. Each of the non-aqueous compositions was a liquid at room temperature.

TABLE 2
Non-aqueous pre-gel concentrate containing TPGS,
benzyl alcohol, and 56.19% non-polar ingredients

| | | mg/0.5 mL | wt % (of |
|---|---|---|---|
| Ingredient | g/batch | serving | composition) |
| TPGS | 6388.13 | 340.7 | 28.39 |
| Benzyl alcohol | 3468.75 | 185 | 15.42 |
| Vitamin E oil (Novatol ™ 5-67) | 768.75 | 41 | 3.42 |
| Borage oil (NLT 22% GLA) | 9375.00 | 500 | 41.67 |
| Alpha-lipoic acid | 1031.25 | 55 | 4.58 |
| Ubiquinol | 281.25 | 15 | 1.25 |
| PQQ | 112.50 | 6 | 0.50 |
| Astaxanthin | 1031.25 | 55 | 4.58 |
| Vitamin K2 (180 mcg MK-7) | 3.75 | 0.2 | 0.017 |
| Vitamin D3 (1 million IU/g) | 39.38 | 2.1 | 0.175 |
| Totals | 22500.00 | 1200 | 100.00 |

TABLE 3
Non-aqueous pre-gel concentrate containing TPGS,
benzyl alcohol, and 54.8% non-polar ingredients

| | | mg/0.5 mL | wt % (of |
|---|---|---|---|
| Ingredient | g/batch | serving | composition) |
| TPGS | 51.90 | 173 | 34.6 |
| Benzyl alcohol | 15.90 | 53 | 10.6 |
| Oleic acid | 30.00 | 100 | 20.0 |
| Vitamin E oil (Novatol ™ 5-67) | 10.20 | 34 | 6.8 |
| Astaxanthin | 42.00 | 140 | 28.0 |
| Totals | 150.00 | 500 | 100.00 |

TABLE 4
Non-aqueous pre-gel concentrate containing TPGS,
benzyl alcohol, and 61.2% non-polar ingredients

| | | mg/0.5 mL | wt % (of |
|---|---|---|---|
| Ingredient | g/batch | serving | composition) |
| TPGS | 42.37 | 564.999 | 28.25 |
| Benzyl alcohol | 15.83 | 211 | 10.55 |
| Oleic acid | 38.63 | 515 | 25.75 |
| Cetyl myristoleate | 45.30 | 604 | 30.20 |
| (Myristin ®) | | | |
| ApresFLEX ® | 7.88 | 105 | 5.25 |
| (boswellia extract) | | | |
| Vitamin D3 (1 million IU/g) | 0.000054 | 0.00072 | 0.000036 |
| Totals | 150.00 | 2000 | 100.00 |

TABLE 5
Non-aqueous pre-gel concentrate containing TPGS, benzyl
alcohol and d-limonene, and 65.55% non-polar ingredients

| | | mg/0.5 mL | wt % (of |
|---|---|---|---|
| Ingredient | g/batch | serving | composition) |
| TPGS | 26.96 | 785 | 26.96 |
| Benzyl alcohol | 2.85 | 83 | 2.85 |
| d-Limonene | 4.64 | 135 | 4.64 |
| Lecithin | 30.05 | 875 | 30.05 |
| Turmeric/curcumin | 2.06 | 60 | 2.06 |
| (95% curcumin) | | | |
| Cetyl myristoleate | 20.74 | 604 | 20.74 |
| (Myristin ®) | | | |
| ApresFLEX ® | 3.61 | 105 | 3.61 |
| (boswellia extract) | | | |
| Perluxan ™ (hops extract) | 9.10 | 265 | 9.10 |
| Vitamin D3 (1 million IU/g) | 0.000025 | 0.00072 | 0.000025 |
| Totals | 100.00 | 2912 | 100.00 |

### Example 4

### Preparation of Non-Aqueous Pre-Gel Concentrates and Soft Gel Capsules Containing TPGS and Non-Polar Ingredients

A. Preparation of Non-Aqueous Pre-Gel Concentrates

Non-aqueous pre-gel concentrates were prepared according to the procedure described above in Example 2. The non-aqueous pre-gel concentrates contained TPGS and one or more non-polar ingredients. Some of the concentrates also contained benzyl alcohol. After formation of the non-aqueous pre-gel concentrates, the compositions were encapsulated in a collagen-containing shell to form a soft gel.

The non-aqueous pre-gel concentrates contained between 9% and 31.78% TPGS up to 91% of one or more non-polar ingredients. Ingredients in the non-aqueous pre-gel concentrates included:

TPGS, a surfactant, that contained a high amount of the dimer form (e.g., 85%-90%) and low amount of the monomer form (e.g., 10%-15%), such as the TPGS prepared according Example 1;

non-aqueous solvents, including benzyl alcohol (Sigma Aldrich, St. Louis, Mo.); and

one or more non-polar ingredients, including: almond oil; an oleic acid compound that contains 70% oleic acid (KIC Chemicals, Inc., New Paltz, N.Y.); a cannabidiol oil that contains 30% cannabidiol; coenzyme Q10-containing compounds, including ubiquinol (Kaneka Ubiquinol®, sold by Kaneka Nutrients, Pasadena, Tex.) and a ubiquinone compound that contains greater than 98% ubidicarenone (ubiquinone), sold under the name Kaneka Q10™ (USP Ubidicarenone; Kaneka Nutrients, L.P., Pasadena, Tex.); natural flavors; pyrroloquinoline quinone (PQQ; such as PureQQ, sold by Nascent Health Science, Allentown, N.J.); an algal oil that contains 35% of the non-polar ingredient DHA and contains 350 mg DHA/g oil (life′sDHA™ S35-O300, sold by DSM Nutritional Products Inc., Kaiseraugst, Switzerland); and a medium chain triglyceride (MCT) oil that contains 98% MCT (sold by Abitec, Janesville, Wis. and Stepan Lipid Nutrition, Maywood, N.J.).

Tables 6-10, below, set forth the ingredients in the non-aqueous pre-gel concentrates, the grams of each ingredient per batch, the amount (mg) of each ingredient per 0.5 mL serving, and the percentage by weight (wt %) of each ingredient by weight of the composition. The non-aqueous pre-gel concentrates were made by a bench-top process according to the methods described above in Example 2. Alternatively, the concentrates can be prepared by scaling up the bench-top process to make larger batch sizes of the non-aqueous pre-gel concentrates. Each of the non-aqueous pre-gel compositions was a liquid at room temperature.

TABLE 6
Non-aqueous pre-gel concentrate containing
TPGS and 80% non-polar ingredients

| | | mg/0.5 mL | wt % (of |
|---|---|---|---|
| Ingredient | g/batch | serving | composition) |
| TPGS | 30.00 | 30.00 | 20.00 |
| Almond oil | 10.00 | 10.00 | 6.67 |
| Oleic acid | 84.00 | 84.00 | 56.00 |
| Cannabidiol oil | 26.00 | 26.00 | 17.33 |
| Totals | 150.00 | 150.00 | 100.00 |

TABLE 7
Non-aqueous pre-gel concentrate containing
TPGS and 91% non-polar ingredients

| | | mg/0.5 mL | wt % (of |
|---|---|---|---|
| Ingredient | g/batch | serving | composition) |
| TPGS | 90.00 | 216.00 | 9.00 |
| Natural flavor | 62.92 | 6.29 | 6.29 |
| Algal oil (35% DHA) | 847.08 | 84.71 | 84.71 |
| Totals | 1000.00 | 2400.00 | 100.00 |

TABLE 8
Non-aqueous pre-gel concentrate containing
TPGS and 89.25% non-polar ingredients

| | | mg/0.5 mL | wt % (of |
|---|---|---|---|
| Ingredient | g/batch | serving | composition) |
| TPGS | 3332.50 | 215 | 10.75 |
| MCT oil | 27667.50 | 1785 | 89.25 |
| Totals | 31000.00 | 2000.00 | 100.00 |

TABLE 9
Non-aqueous pre-gel concentrate containing TPGS,
benzyl alcohol and 63.03% non-polar ingredients

| | | mg/0.5 mL | wt % (of |
|---|---|---|---|
| Ingredient | g/batch | serving | composition) |
| TPGS | 276.66 | 153.70 | 15.37 |
| Benzyl alcohol | 388.80 | 216.00 | 21.60 |
| Oleic acid | 756.54 | 420.30 | 42.03 |
| Ubiquinone | 378.00 | 210.00 | 21.00 |
| Totals | 1800.00 | 1000.00 | 100.00 |

TABLE 10
Non-aqueous pre-gel concentrate containing TPGS,
benzyl alcohol and 41.05% non-polar ingredients

| | | mg/0.5 mL | wt % (of |
|---|---|---|---|
| Ingredient | g/batch | serving | composition) |
| TPGS | 95.35 | 190.70 | 31.78 |
| Benzyl alcohol | 81.50 | 163.00 | 27.17 |
| Oleic acid | 84.65 | 169.30 | 28.22 |
| PQQ | 12.00 | 24.00 | 4.00 |
| Ubiquinol | 26.50 | 53.00 | 8.83 |
| Totals | 300.00 | 600.00 | 100.00 |

B. Preparation of Soft-Gel Capsules

Soft gel capsules containing each of the pre-gel concentrates set forth in Tables 6-10, above, were prepared by Captek Softgel International (Cerritos, Calif.) from a gelatin or other suitable material using standard methods. The outer capsule was formed using a polar emulsion and gelatin or other known gel replacement, at an appropriate ratio to form the gel capsule. The capsule was of a size to encompass a dosage amount for direct oral consumption and was filled with an appropriate selected dosage of a composition as described above.

Since modifications will be apparent to those of skill in this art, it is intended that this invention be limited only by the scope of the appended claims.

1. A non-aqueous pre-gel concentrate composition, comprising:
a polyethylene glycol (PEG) derivative of vitamin E in an amount between 5% and 50%, inclusive, by weight of the concentrate;
a non-polar ingredient, other than the PEG derivative of vitamin E, in an amount between about 40% to about 90%, inclusive, by weight of the concentrate, wherein the non-polar ingredient is or contains one or more non-polar compounds; and
an ingestible non-aqueous solvent in an amount between 2% and 40%, by weight of the concentrate.

2. The concentrate of claim 1, wherein the amount of non-polar ingredient in the concentrate is greater than the amount of PEG derivative of vitamin E.

3. The concentrate of claim 1, wherein the ratio of the amount of non-polar ingredient to PEG derivative of vitamin E is about 1.3:1 to 4:1.

4. The concentrate of claim 1, wherein the PEG derivative of vitamin E contains a PEG moiety having a molecular weight from between or between about 100 Da and 20,000 Da.

5. The concentrate of claim 1, wherein the PEG derivative of vitamin E is selected from among tocopheryl polyethylene glycol succinate (TPGS), tocopheryl polyethylene glycol sebacate, tocopheryl polyethylene glycol dodecanodioate, tocopheryl polyethylene glycol suberate, tocopheryl polyethylene glycol azelaate, tocopheryl polyethylene glycol citraconate, tocopheryl polyethylene glycol methylcitraconate, tocopheryl polyethylene glycol itaconate, tocopheryl polyethylene glycol maleate, tocopheryl polyethylene glycol glutarate, tocopheryl polyethylene glycol glutaconate, tocopheryl polyethylene glycol fumarate, tocopheryl polyethylene glycol phthalate, tocotrienol polyethylene glycol succinate, tocotrienol polyethylene glycol sebacate, tocotrienol polyethylene glycol dodecanodioate, tocotrienol polyethylene glycol suberate, tocotrienol polyethylene glycol azelaate, tocotrienol polyethylene glycol citraconate, tocotrienol polyethylene glycol methylcitraconate, tocotrienol polyethylene glycol itaconate, tocotrienol polyethylene glycol maleate, tocotrienol polyethylene glycol glutarate, tocotrienol polyethylene glycol glutaconate, tocotrienol polyethylene glycol fumarate and tocotrienol polyethylene glycol phthalate, TPGS analogs and TPGS homologs.

6. The concentrate of claim 1, wherein the PEG derivative of vitamin E is tocopheryl polyethylene glycol succinate (TPGS).

7. The concentrate of claim 1, wherein the PEG derivative of vitamin E is tocopheryl polyethylene glycol succinate 1000 (TPGS 1000).

8. The concentrate of claim 1, wherein the PEG derivative of vitamin E is present in an amount between 20% and 40%, inclusive, by weight of the concentrate.

9. The concentrate of claim 1, wherein the non-polar ingredient is selected from among polyunsaturated fatty acids (PUFAs), non-essential fatty acids, phospholipids, coenzyme Q compounds, flavonoids, carotenoids, micronutrients, Boswellia extracts, alkaloids, hops-containing compounds, antioxidants, oil-soluble vitamins, and mixtures thereof.

10. The concentrate of claim 9, wherein the PUFA is selected from among omega-3 fatty acids, omega-6 fatty acids, omega-9 fatty acids, and conjugated fatty acids.

11. The concentrate of claim 1, wherein the non-polar ingredient is selected from among one or more of:
a coenzyme Q10 that is selected from among ubiquinol, ubidecarenone, and ubisemiquinone;
an oil-soluble vitamin that is selected from among vitamin B12, vitamin D3, vitamin A palmitate, vitamin E, vitamin B1, vitamin B3, vitamin B5, vitamin B6, vitamin C, vitamin K2, and mixtures thereof;
a carotenoid-containing compound that is selected from among astaxanthin, lycopene, lutein, zeaxanthin, and mixtures thereof;
a Boswellia extract-containing compound that is a Boswellia serrata extract that contains acetyl-11-keto-β-boswellic acid (AKBA);
a hops-containing compound that contains a supercritical extract of hops cones that includes a minimum of 30% alpha-acids and 10% beta-acids (including lupulone and colupulone);
an antioxidant that is selected from among alpha-lipoic acid, pyrroloquinoline quinone (PQQ), a turmeric/curcumin composition that is 95% curcumin, and mixtures thereof;
an omega-5 fatty acid derivative that is cetyl myristoleate (CMO); and
a phospholipid that is a phosphatidylcholine.

12. The concentrate of claim 1, wherein the non-polar ingredient is present in an amount of from 40%-70%, by weight of the concentrate.

13. The concentrate of claim 1, wherein the non-polar ingredient is present in an amount of from 55%-70%, by weight of the concentrate.

14. The concentrate of claim 1, wherein the PEG derivative of vitamin E is TPGS and is present in an amount between 25%-40%, by weight of the concentrate.

15. The concentrate of claim 1, wherein the non-aqueous solvent is selected from among ingestible alcohols, alcohol derivatives, alkanes, aromatic alcohols, aromatic ethers, aromatic esters, haloalkanes, ethers, esters, ketones, organic solvents of natural origin, lactams, alkylene glycols, glycerol, natural oils, saturated and non-saturated fatty acids and mixtures thereof.

16. The concentrate of claim 1, wherein the non-aqueous solvent is an alcohol, an alcohol derivative, a hydrocarbon, or mixtures thereof.

17. The concentrate of claim 1, wherein the non-aqueous solvent is selected from among benzyl alcohol, benzyl benzoate, d-limonene and mixtures thereof.

18. The concentrate of claim 1, wherein the non-aqueous solvent is benzyl alcohol.

19. The concentrate of claim 1, wherein the non-aqueous solvent is present in an amount of at least 5%, up to and including 20%, or in an amount of at least 7%, up to and including 17%.

20. A concentrate of claim 1, selected from among:
a) a concentrate, comprising:
a polyethylene glycol (PEG) derivative of vitamin E that is tocopheryl polyethylene glycol succinate (TPGS) or a water-soluble vitamin E derivative mixture that is a high-dimer PEG derivative of vitamin E mixture that is a high-dimer TPGS mixture, in an amount between 25% and 35%, inclusive, by weight of the concentrate;
a non-aqueous solvent selected from among benzyl alcohol, d-limonene, or a mixture thereof, in an amount between 7% and 16%, inclusive, by weight of the concentrate; and
a non-polar ingredient in an amount between 53% and 66%, inclusive, by weight of the concentrate, wherein the non-polar ingredient is selected from among one or more of:
a coenzyme Q10 that is selected from among ubiquinol, ubidecarenone, and ubisemiquinone;
an oil-soluble vitamin that is selected from among vitamin B12, vitamin D3, vitamin A palmitate, vitamin E, vitamin B1, vitamin B3, vitamin B5, vitamin B6, vitamin C, vitamin K2, and mixtures thereof;
a carotenoid-containing compound that is selected from among astaxanthin, lycopene, lutein, zeaxanthin, and mixtures thereof;
a Boswellia extract-containing compound that is a Boswellia serrata extract that contains acetyl-11-keto-β-boswellic acid (AKBA);
a hops-containing compound that includes a minimum of 30% alpha-acids and 10% beta-acids;
an antioxidant that is selected from among alpha-lipoic acid, pyrroloquinoline quinone (PQQ), a turmeric/curcumin composition that is 95% curcumin, and mixtures thereof;
an omega-5 fatty acid derivative that is cetyl myristoleate (CMO); and
a phospholipid that is a phosphatidylcholine;
b) a concentrate, comprising:
a polyethylene glycol (PEG) derivative of vitamin E that is tocopheryl polyethylene glycol succinate (TPGS) or a water-soluble vitamin E derivative mixture that is a high-dimer PEG derivative of vitamin E mixture that is a high-dimer TPGS mixture, in an amount between 25% and 30%, inclusive, by weight of the concentrate;
a non-aqueous solvent that is a mixture of benzyl alcohol and d-limonene, in an amount between 7% and 16%, inclusive, by weight of the concentrate; and
a non-polar ingredient in an amount between 60% and 66%, inclusive, by weight of the concentrate, wherein the non-polar ingredient comprises a mixture of vitamin D3, a Boswellia serrata extract that contains acetyl-11-keto-β-boswellic acid (AKBA), a hops-containing compound that contains a supercritical extract of hops cones that includes a minimum of 30% alpha-acids and 10% beta-acids, a turmeric/curcumin composition that is 95% curcumin, cetyl myristoleate (CMO), and phosphatidylcholine;
c) a concentrate, comprising:
a polyethylene glycol (PEG) derivative of vitamin E that is tocopheryl polyethylene glycol succinate (TPGS) or a water-soluble vitamin E derivative mixture that is a high-dimer PEG derivative of vitamin E mixture that is a high-dimer TPGS mixture, in an amount between 25% and 30%, inclusive, by weight of the concentrate;
a non-aqueous solvent that is benzyl alcohol, in an amount between 12% and 17%, inclusive, by weight of the concentrate; and
a non-polar ingredient in an amount between 53% and 58%, inclusive, by weight of the concentrate, wherein the non-polar ingredient comprises a mixture of vitamin E oil, vitamin D3, vitamin K2, borage oil, alpha-lipoic acid, ubiquinol, pyrroloquinoline quinone (PQQ), and astaxanthin;
d) a concentrate, comprising:
a polyethylene glycol (PEG) derivative of vitamin E that is tocopheryl polyethylene glycol succinate (TPGS) or a water-soluble vitamin E derivative mixture that is a high-dimer PEG derivative of vitamin E mixture that is a high-dimer TPGS mixture, in an amount between 28% and 35%, inclusive, by weight of the concentrate;
a non-aqueous solvent that is benzyl alcohol, in an amount between 10% and 12%, inclusive, by weight of the concentrate; and
a non-polar ingredient in an amount between 52% and 62%, inclusive, by weight of the concentrate, wherein the non-polar ingredient comprises oleic acid, and optionally an ingredient selected from among vitamin E oil and astaxanthin, cetyl myristoleate, vitamin D3, and a Boswellia extract-containing compound that is a Boswellia serrata extract that contains acetyl-11-keto-β-boswellic acid (AKBA).

21. The concentrate of claim 1, further comprising a co-surfactant selected from a phospholipid or a sugar-derived surfactant.

22. A capsule composition, comprising the non-aqueous pre-gel concentrate of claim 1 encapsulated in a shell or coating.

23. The capsule composition of claim 22, wherein the capsule shell or coating is a soft gel.

24. The capsule composition of claim 23, wherein the shell or coating is a gelatin or gelatin substitute shell or coating.

25. The capsule composition of claim 24, wherein the gelatin or gelatin substitute is selected from among natural gelatin, synthetic gelatin, pectin, casein, collagen, protein, modified starch, polyvinyl pyrrolidone, acrylic, natural or synthetic polymers, a cellulose derivative that is hydroxypropyl methylcellulose (HPMC), seaweed extract, and combinations thereof.

26. The capsule composition of claim 22, wherein the volume the shell or coating can hold is between about 0.1-1 mL or between about 10 mg to 1000 mg.

27. A capsule composition, comprising the non-aqueous pre-gel concentrate of claim 2 encapsulated in a shell or coating.

28. A capsule composition, comprising the non-aqueous pre-gel concentrate of claim 3 encapsulated in a shell or coating.

29. A capsule composition, comprising the non-aqueous pre-gel concentrate of claim 6 encapsulated in a shell or coating.

30. A capsule composition, comprising the non-aqueous pre-gel concentrate of claim 7 encapsulated in a shell or coating.

31. A capsule composition, comprising the non-aqueous pre-gel concentrate of claim 8 encapsulated in a shell or coating.

32. A capsule composition, comprising the non-aqueous pre-gel concentrate of claim 9 encapsulated in a shell or coating.

33. A capsule composition, comprising the non-aqueous pre-gel concentrate of claim 10 encapsulated in a shell or coating.

34. A capsule composition, comprising the non-aqueous pre-gel concentrate of claim 14 encapsulated in a shell or coating.

35. A capsule composition, comprising the non-aqueous pre-gel concentrate of claim 16 encapsulated in a shell or coating.

36. A capsule composition, comprising the non-aqueous pre-gel concentrate of claim 17 encapsulated in a shell or coating.

37. A capsule composition, comprising the non-aqueous pre-gel concentrate of claim 18 encapsulated in a shell or coating.

38. A capsule composition, comprising the non-aqueous pre-gel concentrate of claim 19 encapsulated in a shell or coating.

39. A capsule composition, comprising the non-aqueous pre-gel concentrate of claim 20 encapsulated in a shell or coating.

40. A method of making the pre-gel concentrate of claim 1, comprising:
a) mixing and heating the ingredients in a vessel;
b) homogenizing the ingredients; and
c) cooling the mixed ingredients to produce the concentrate.

41. The method of claim 40, further comprising introducing the pre-gel concentrate into a soft gel shell or capsule.

42. A method of providing a non-polar compound to a subject, comprising administering a capsule of claim 22 to a subject.
